(19) United States
(12) Patent Application Publication
(10) Pub. No.: US 2023/0357793 A1
(43) Pub. Date: Nov. 9, 2023

(54) ONCOLYTIC HERPES SIMPLEX VIRUSES (HSV) EXPRESSING IMMUNOMODULATORY FUSION PROTEINS

(71) Applicant: SORRENTO THERAPEUTICS, INC., San Diego, CA (US)

(72) Inventors: Robert ALLEN, San Diego, CA (US); Colin POWERS, San Diego, CA (US); Gunnar KAUFMANN, San Diego, CA (US); Joe CONNER, San Diego, CA (US)

(21) Appl. No.: 18/002,336

(22) PCT Filed: Jun. 25, 2021
(86) PCT No.: PCT/US2021/039193

§ 371 Date: Dec. 19, 2022

Related U.S. Application Data
(60) Provisional application No. 63/044,818, filed on Jun. 26, 2020.

(51) Int. Cl. C12N 15/86; C07K 16/28; C07K 14/71; C07K 14/54; A61P 35/00

(52) U.S. Cl. CPC C12N 15/86; C07K 16/2827; C07K 16/2818; C07K 14/71; C07K 14/5434; A61P 35/00; C07K 2317/622; C12N 2710/16643; C07K 2319/33

(57) ABSTRACT

Recombinant oncolytic viruses that produce and secrete novel immunomodulatory fusion proteins are described. The fusion proteins encode a single chain variable fragment antibody (ScFv) that specifically binds PD-1 OR PD-L1 fused via an antibody Fc region to the ectodomain of the TGFβ receptor II (TGFβRII_{ecto}). The immunomodulatory fusion proteins have dual function: blocking inhibitory pathways mediated by PD-1/PD-L1 and blocking the immune-dampening activity of TGFβ. In addition, dual gene oncolytic herpes simplex viruses (HSVs) are provided that include, in addition to a gene encoding an ScFv-Fc-TGFβRII_{ecto} fusion protein, a gene encoding IL12, a T cell stimulatory factor.

This patent application claims priority to U.S. provisional application 63/044,818 filed Jun. 26, 2020, the contents of which is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 23, 2021, is named 087735_0140_SL.txt and is 126,354 bytes in size.

### TECHNICAL FIELD

The invention relates to oncolytic viruses engineered to express proteins that modulate the immune response and their use in treating cancer. More specifically, the disclosure provides recombinant oncolytic herpes simples viruses that include genetic constructs encoding proteins that inhibit immune system regulators.

### BACKGROUND

Oncolytic viruses are viruses that selectively infect and lyse cancer cells. Oncolytic viruses have been the subject of clinical trials for the treatment various cancers, including melanoma, glioma, head and neck cancer, ovarian cancer, lung cancer, liver cancer, bladder cancer, prostate cancer, and pancreatic cancer (Aghi & Martuza (2005) Oncogene 24:7802-7816). Multiple clinical trials have demonstrated the safety of oncolytic herpes simplex viruses (HSVs) attenuated in their ability to replicate in normal cells by deletion of at least one copy of the gene encoding ICP34.5 (Rampling et al. (2000) Gene Therapy 7:859-866; Papanastassiou et al. (2002) Gene Therapy 9:398-406; Makie et al. (2001) Lancet 357:525-526; Markert et al. (2000) Gene Therapy 7:867-874; Markert et al. (2009) Molecular Therapy 17:199-207; Senzer et al. (2009) J Clin Oncol 27:5763-5771).

In addition to directly attacking the tumor by lysing cancer cells, oncolytic HSVs can induce an anti-tumor immune response in the patient (Papanastassiou et al. (2002); Markert et al. (2009); Senzer et al. (2009)) as viral antigens are expressed on infected cancer cells and tumor antigens are released when cancer cells are lysed. Viruses also engage mediators of the innate immune response as part of the host recognition of viral infection resulting in an inflammatory response (Hu et al. (2006) Clin Cancer Res. 12:6737-6747). These immune responses to treatment with oncolytic viruses may provide a systemic benefit to cancer patients resulting in the suppression of tumors which have not been infected with the virus, including metastatic tumors, and may prevent disease recurrence.

Tumor cells can however escape destruction by the immune system by engaging inhibitory immune checkpoint pathways (Pardoll (2012) Nature Reviews Cancer Vol. 12:252-264; Darvin et al. (2018) Exp Mol Med 50:1-11). Inhibitory immune checkpoint pathways, such as those mediated by interactions of immune checkpoint proteins PD-1, PD-L1, CTLA-4. LAG-3, TIM3, and TIGIT counteract immune system activation to prevent autoimmune responses. Tumor cells can take advantage of these inhibitory pathways by expressing immune checkpoint proteins that interact with their counterparts on T cells, resulting in de-activation of the T cells and shutting down of the anti-tumor immune response. Immune checkpoint proteins inhibit the activation or function of T-cells to regulate the intensity and duration of immune responses and maintain self-tolerance. Numerous immune checkpoint proteins are known, such as PD-1 (Programmed Death 1) with its ligands PD-L1 and PD-L2, CTLA-4 (Cytotoxic T-Lymphocyte-Associated protein 4) and its ligands CD80 and CD86; TIM-3 (T-cell Immunoglobulin domain and Mucin domain 3), LAG-3 (Lymphocyte Activation Gene-3), TIGIT (T cell immunoreceptor with Ig and ITIM domains), BTLA (CD272 or B and T Lymphocyte Attenuator), and VISTA (V-domain immunoglobulin suppressor of T-cell activation) (Pardoll (2012) Nature Reviews Cancer 12:252-264; Borcherding et al. (2018) J Mol Biol 430:2014-2029).

In 2015 Talimogene laherparepvec (“TVec”), an HSV derived from a clinical HSV strain by functional deletion of two genes (encoding ICP34.5 and ICP47) and insertion of a gene encoding granulocyte macrophage colony-stimulating factor (GM-CSF), became the first oncolytic immunotherapy approved for use in the United States when it was approved for the treatment of melanoma. The overall response rate for patients having Stage IIIB to Stage IV melanoma in the Phase III study was 26% (Andtbacka et al. (2015) J Clin Oncol 33:2780-2788). There is a need to increase the effectiveness of oncolytic viral therapy and extend its use to other types of cancer.

### SUMMARY

Disclosed herein are recombinant oncolytic herpes simplex viruses (HSVs) designed to produce and secrete novel immunomodulatory proteins in the form of ScFv-Fc-TGFβtrap fusion proteins that include a single chain variable fragment antibody (ScFv) that specifically binds an immune checkpoint molecule such as PD-1 or PD-L1 fused to the ectodomain of the TGFβ receptor II via an antibody Fc region. The immunomodulatory fusion proteins are bifunctional, blocking inhibitory pathways mediated by the immune checkpoint molecule and preventing engagement of TGFβ with its receptor. Further provided are recombinant HSVs that include dual gene constructs that include, in addition to a gene encoding a dual function ScFv-Fc-TGFβtrap protein, a gene encoding interleukin 12 (IL12), an immune stimulatory cytokine. The engineered oncolytic HSVs selectively infect and replicate in tumor cells, allowing for production and secretion of the immunomodulatory molecules at the tumor site. Also provided herein are anti-PD-1 or anti-PD-L1 ScFv-Fc-TGFβtrap proteins and compositions that include such proteins, including virus-free conditioned media that includes an ScFv-Fc-TGFβtrap protein. A protein composition as provided herein may optionally include, in addition to an ScFv-Fc-TGFβtrap protein, an immune activator such as IL12. The recombinant oncolytic HSVs and/or compositions that include proteins produced and secreted by cells infected with the recombinant oncolytic HSVs can be delivered to a subject for the treatment of cancer. The subject may be a human cancer patient or may be a non-human animal such as, for example, a dog, cat, or horse.

In a first aspect, provided herein are engineered oncolytic herpes simplex viruses (HSVs) that include nucleic acid constructs that comprise a sequence encoding a fusion protein comprising an ScFv that binds an immune checkpoint protein, where the ScFv is fused to the ectodomain of the TGFβ receptor II (TGFβRII_{ecto}) via an Fc antibody region. Such fusion proteins are referred to herein as ScFv-Fc-TGFβtrap fusion proteins or simply ScFv-Fc-TGFβtrap proteins and may also be referred to as ScFv-Fc-TGFβRII_{ecto} [fusion] proteins.

The ScFv of the ScFv-Fc-TGFβtrap fusion protein specifically binds an immune checkpoint protein such as PD-1 or PD-L1 to prevent engagement of the immune checkpoint protein with its receptor or ligand. In some embodiments the ScFv moiety of the fusion protein is derived from a monoclonal antibody that specifically binds PD-1, such as, for example, the BB9 anti-PD-1 antibody, the RG1H10 anti-PD-1 antibody, or pembrolizumab. For example, the ScFv may be derived from the BB9 antibody and include a heavy chain variable region sequence having heavy chain CDRs (HC-CDRs) having the amino acid sequences of SEQ ID NO:63 (HC-CDR1), SEQ ID NO:64 (HC-CDR2), and SEQ ID NO:65 (HC-CDR3), and a light chain variable region sequence having light chain CDRs (LC-CDRs) having the amino acid sequences of SEQ ID NO:66 (LC-CDR1), SEQ ID NO:67 (LC-CDR2), and SEQ ID NO:68 (LC-CDR3). The BB9-derived anti-PD-1 scFv can include a heavy chain variable region having at least 95% identity to SEQ ID NO:8 and a light chain variable region sequence having at least 95% identity to SEQ ID NO:9. In certain embodiments, the ScFv moiety of the fusion protein may comprise SEQ ID NO:11 or a sequence having at least 95% identity to SEQ ID NO:11. In further embodiments, the ScFv may be derived from the RG1H10 antibody and include a heavy chain variable region sequence having at least 95% identity to SEQ ID NO:12 and a light chain variable region sequence having at least 95% identity to SEQ ID NO:13. In some embodiments, the ScFv moiety of the fusion protein may comprise SEQ ID NO:15 or a sequence having at least 95% identity to SEQ ID NO:15. In additional embodiments, the ScFv may be derived from pembrolizumab and include a heavy chain variable region sequence having at least 95% identity to SEQ ID NO:16 and a light chain variable region sequence having at least 95% identity to SEQ ID NO:17. In some embodiments, the ScFv moiety of the fusion protein may comprise SEQ ID NO:19 or a sequence having at least 95% identity to SEQ ID NO:19.

In some embodiments the ScFv moiety of the ScFv-Fc-TGFβtrap fusion protein is derived from a monoclonal antibody that specifically binds PD-L1, such as the Combi5 anti-PD-L1 antibody, the H6B1LEM anti-PD-L1 antibody, or avelumab. For example, the ScFv may be derived from the Combi5 antibody and include a heavy chain variable region sequence having at least 95% identity to SEQ ID NO:20 and a light chain variable region sequence having at least 95% identity to SEQ ID NO:21. In certain embodiments, the ScFv moiety of the fusion protein may comprise SEQ ID NO:23 or a sequence having at least 95% identity to SEQ ID NO:23. In further embodiments, the ScFv may be derived from the H6B1LEM antibody and include a heavy chain variable region sequence having at least 95% identity to SEQ ID NO:24 and a light chain variable region sequence having at least 95% identity to SEQ ID NO:25. In some embodiments, the ScFv moiety of the fusion protein may comprise SEQ ID NO:27 or a sequence having at least 95% identity to SEQ ID NO:27. In additional embodiments, the ScFv may be derived from avelumab and include a heavy chain variable region sequence having at least 95% identity to SEQ ID NO:28 and a light chain variable region sequence having at least 95% identity to SEQ ID NO:29. In some embodiments, the ScFv moiety of the fusion protein may comprise SEQ ID NO:31 or a sequence having at least 95% identity to SEQ ID NO:31.

The TGFβ trap moiety of the ScFv-Fc-TGFβtrap fusion protein comprises the ectodomain of the TGFβ receptor II (TGFβRII). The TGFβ ectodomain can be the ectodomain of human TGFβRII (SEQ ID NO:7) or can be a polypeptide sequence having at least 95% identity to SEQ ID NO:7. The ScFv moiety of the fusion protein is attached to the TGFβRII ectodomain via an Fc antibody region. The Fc region can be an Fc region of an IgG1 or IgG4 antibody, and can be a human IgG1 or IgG4 Fc region or a variant thereof, for example, can be an Fc region comprising SEQ ID NO:2 or a sequence having at least 95% identity to SEQ ID NO:2, or can be an Fc region comprising SEQ ID NO:5 or a sequence having at least 95% identity to SEQ ID NO:5. A peptide linker can optionally connect the Fc region to the TGFβRII ectodomain, such as for example, a flexible peptide linker such as (GGGGS)n (SEQ ID NO: 61), e.g., SEQ ID NO:56.

In various embodiments an ScFv-Fc-TGFβtrap fusion protein encoded by a nucleic acid construct of an oncolytic virus can include a signal peptide for secretion of the fusion protein from the cell. The signal peptide can be any that directs secretion and can preferably be N-terminal to the ScFv of the fusion protein. One example of a signal peptide that may be used at the N-terminus of an ScFv-Fc-TGFβtrap fusion protein is SEQ ID NO:34.

The nucleic acid construct that comprises a sequence encoding the ScFv-Fc-TGFβtrap fusion protein can further include a promoter. The promoter is operably linked to the ScFv-Fc-TGFβtrap-encoding sequence and can be a promoter functional in a mammalian cell, such as a human cell. Examples of mammalian promoters that may be operably linked to an ScFv-Fc-TGFβtrap gene include, without limitation, a CMV promoter, an EF1a promoter, an HTLV promoter, an EF1α/HTLV hybrid promoter, or a JeT promoter.

An oncolytic virus as provided herein that encodes an ScFv-Fc-TGFβtrap fusion protein can further include one or more additional transgenes. In various embodiments, at least one additional transgene can be a immune activating cytokine, such as, for example, GM-CSF, IL2, IL12, IL15, IL18, IL21, IL24, a type I interferon, a type III interferon, interferon gamma, or TNFα. In some embodiments an oncolytic virus as provided herein that encodes an ScFv-Fc-TGFβtrap fusion protein can further include a transgene encoding IL12.

A second aspect provided herein is engineered HSVs that include two transgenes: a first gene encoding an ScFv-Fc-TGFβtrap fusion protein as disclosed above, in which the ScFv of the fusion protein binds an immune checkpoint inhibitor such as PD-1 or PD-L1, and a second gene encoding IL12. The IL12 gene can encode a mammalian IL12, for example, a murine IL12 or a human IL12. In its mature functional form IL12 is a heterodimer of two polypeptides, the p40 and the p35 subunits. A recombinant HSV as provided herein that includes a gene for expressing IL12 can include a sequence encoding the p40 polypeptide chain and a sequence encoding the p35 polypeptide chain, in which each sequence is independently operably linked to a separate promoter, or alternatively, the two IL12 subunits can be operably linked to the same promoter and separated by a self-cleaving 2A sequence or an IRES for the production of two polypeptide chains. In further embodiments, the p40 subunit-encoding sequence can be linked to the p35-encoding sequence via a sequence encoding a peptide linker that allows for production of a single polypeptide encoding both subunits. For example, the IL12 gene can encode a single polypeptide (e.g., SEQ ID NO:52) comprising a human IL12 p40 subunit attached to a human IL12 p35 subunit via a 2× elastin linker (SEQ ID NO:55).

The two transgenes of the recombinant oncolytic HSVs provided herein (i.e., the ScFv-Fc-TGFβtrap and the IL12 gene) can be provided in a single construct, i.e., a dual gene construct, and the ScFv-Fc-TGFβtrap gene and IL12 can be operably linked to a single promoter, or each gene can be operably linked to separate promoters. Where a single promoter is used, the coding regions of each gene can be linked, for example by an IRES or 2A “self-cleaving peptide” sequence.

Some embodiments of an ScFv-Fc-TGFβtrap fusion protein encoded by an oncolytic HSV as provided herein are ScFv-Fc-TGFβtrap fusion proteins in which the ScFv specifically binds PD-1 and is derived from monoclonal antibody BB9, monoclonal antibody RG1H10, or pembrolizumab, and the Fc region is an IgG1 Fc or IgG4 Fc. For example, the anti-PD-1 ScFv-Fc-TGFβtrap fusion protein can have the sequence of SEQ ID NO:40, SEQ ID NO:42, or SEQ ID NO:44 or can have a sequence having at least 95% identity to any of SEQ ID NO:40, SEQ ID NO:42, or SEQ ID NO:44. Other examples of an ScFv-Fc-TGFβtrap fusion protein are ScFv-Fc-TGFβtraps in which the ScFv specifically binds PD-L1 and is derived from monoclonal antibody Combi5, monoclonal antibody H6B1LEM, or avelumab, and the Fc region is an IgG1 Fc or IgG4 Fc. For example, the anti-PD-L1 ScFv-Fc-TGFβtrap fusion protein can have the sequence of SEQ ID NO:46, SEQ ID NO:48, or SEQ ID NO:50 or can have a sequence having at least 95% identity to any of SEQ ID NO:46, SEQ ID NO:48, or SEQ ID NO:50. Specifically but not exclusively provided by the disclosure are recombinant HSVs encoding any of the exemplified ScFv-Fc-TGFβtraps, including dual gene recombinant HSVs encoding any of the exemplified ScFv-Fc-TGFβtraps and also encoding IL12.

The recombinant HSV according to any of the embodiments provided herein may be an HSV-1 strain or an HSV-2 strain, and in some preferred embodiments is derived from HSV-1 strain F, HSV-1 strain KOS, HSV-1 strain JS1, or HSV-1 strain 17. In various embodiments the HSV-1 strain does not include a functional ICP34.5-encoding gene. For example, the HSV into which the ScFv-Fc-TGFβtrap gene and, optionally, the IL12 gene is inserted can be Seprehvec®, an HSV-1 strain 17-derived HSV that lacks a functional ICP34.5-encoding gene (the RL1 gene), where both copies of the ICP34.5-encoding gene have a 695 bp deletion. The site of the deletion in the ICP34.5-encoding gene is also the insertion site for the constructs provided herein. Thus, in various exemplary embodiments the recombinant HSVs provided herein are Seprehvec viruses that include a transgene encoding an ScFv-Fc-TGFβtrap and, optionally, a transgene encoding IL12 inserted at each ICP34.5 gene locus, where both ICP34.5-encoding genes of the Seprehvec HSV are inactivated, for example by a 695 bp deletion that extends from upstream of the ICP34.5-encoding sequence into the coding region, such that a major portion of the RL-1 gene is deleted.

A further aspect of the disclosure is a pharmaceutical composition comprising any of the recombinant oncolytic HSVs provided herein in a pharmaceutically acceptable carrier or solution. The pharmaceutical virus preparation can include the recombinant HSV at a titer of about 10⁶ pfu per ml or higher, for example, a titer of about 10⁷ pfu per ml or a titer of 108 pfu per ml or higher. The pharmaceutical recombinant HSV composition can be packaged for injection or infusion, for example in vials. The virus can be provided in a buffer that can optionally include a cryoprotectant such as, for example, glycerol. The pharmaceutical composition can be provided as a frozen composition.

Another aspect of the disclosure is a method of treating cancer using a recombinant HSV that encodes an ScFv-Fc-TGFβtrap. The method can include administering a recombinant HSV that comprises a nucleic acid construct encoding an ScFv-Fc-TGFβtrap as provided herein to a subject having cancer. In some embodiments the cancer may be a solid tumor. The recombinant HSV can be any disclosed herein, such as, for example, any that encodes an ScFv-Fc-TGFβtrap that is able to bind an immune checkpoint inhibitor such as PD-1 or PD-L1. The subject may be a human or may be a non-human animal such as, for example, a dog, cat, cow, bull, or horse. The cancer can be without limitation, bladder, bone, breast, eye, stomach, head and neck, kidney, liver, lung, ovarian, pancreatic, prostate, skin, or uterine cancer, a mesothelioma, a glioma, a neurocytoma, or a chondrosarcoma. The administering can be by any means and can be, as nonlimiting examples, parenteral, systemic, intracavitary (e.g, intrapleural, intraperitoneal), peritumoral, or intratumoral, and may be by injection, intravenous or intra-arterial infusion, or other delivery means. Injection can be, for example, parenteral, subcutaneous, intramuscular, intravenous, intra-arterial, intratumoral, or peritumoral. The treatment regimen may include more than one administration of the virus and can include multiple dosings over a period of days, weeks, or months. In some embodiments the ScFv-Fc-TGFβtrap encoded by the HSV used in the methods is an anti-PD-1 ScFv-Fc-TGFβtrap, for example, an anti-PD-1 ScFv-Fc-TGFβtrap having an scFv that includes heavy chain CDRs and light chain CDRs of antibody BB9, i.e., a heavy chain variable region sequence having heavy chain CDRs (HC-CDRs) having the amino acid sequences of SEQ ID NO:63 (HC-CDR1), SEQ ID NO:64 (HC-CDR2), and SEQ ID NO:65 (HC-CDR3), and a light chain variable region sequence having light chain CDRs (LC-CDRs) having the amino acid sequences of SEQ ID NO:66 (LC-CDR1), SEQ ID NO:67 (LC-CDR2), and SEQ ID NO:68 (LC-CDR3), and in some examples the scFv of the anti-PD-1 ScFv-Fc-TGFβtrap protein comprises a heavy chain variable region sequence having at least 95% identity to SEQ ID NO:8 and a light chain variable region sequence having at least 95% identity to SEQ ID NO:9. In further examples the ScFv-Fc-TGFβtrap encoded by the HSV used in the methods is an anti-PD-L1 ScFv-Fc-TGFβtrap, and in some embodiments has a heavy chain variable region sequence having at least 95% identity to SEQ ID NO:20 and a light chain variable region sequence having at least 95% identity to SEQ ID NO:21.

Treating cancer can use a recombinant HSV that encodes an ScFv-Fc-TGFβtrap as disclosed herein and further encodes IL12. The method can include administering a recombinant HSV that comprises a nucleic acid construct encoding an ScFv-Fc-TGFβtrap as provided herein and that also encodes IL12 to a subject having cancer. In some embodiments the cancer may be a solid tumor. The recombinant HSV can be any disclosed herein, such as, for example, any that encodes an ScFv-Fc-TGFβtrap that is able to bind an immune checkpoint inhibitor such as PD-1 or PD-L1. The subject may be a human or may be a non-human animal such as, for example, a dog, cat, cow, bull, or horse. The cancer can be without limitation, bladder, bone, breast, eye, stomach, head and neck, kidney, liver, lung, ovarian, pancreatic, prostate, skin, or uterine cancer, a mesothelioma, a glioma, a neurocytoma, or a chondrosarcoma. The administering can be by any means and can be, as nonlimiting examples, parenteral, systemic, intracavitary (e.g, intrapleural, intraperitoneal), peritumoral, or intratumoral, and may be by injection, intravenous or intra-arterial infusion, or other delivery means. Injection can be, for example, parenteral, subcutaneous, intramuscular, intravenous, intra-arterial, intratumoral, or peritumoral. The treatment regimen may include more than one administration of the virus and can include multiple dosings over a period of days, weeks, or months. In some embodiments the ScFv-Fc-TGFβtrap encoded by the HSV used in the methods is an anti-PD-1 ScFv-Fc-TGFβtrap, for example, an anti-PD-1 ScFv-Fc-TGFβtrap having an scFv that includes heavy chain CDRs and light chain CDRs of antibody BB9, i.e., a heavy chain variable region sequence having heavy chain CDRs (HC-CDRs) having the amino acid sequences of SEQ ID NO:63 (HC-CDR1), SEQ ID NO:64 (HC-CDR2), and SEQ ID NO:65 (HC-CDR3), and a light chain variable region sequence having light chain CDRs (LC-CDRs) having the amino acid sequences of SEQ ID NO:66 (LC-CDR1), SEQ ID NO:67 (LC-CDR2), and SEQ ID NO:68 (LC-CDR3), and in some examples the scFv of the anti-PD-1 ScFv-Fc-TGFβtrap protein comprises a heavy chain variable region sequence having at least 95% identity to SEQ ID NO:8 and a light chain variable region sequence having at least 95% identity to SEQ ID NO:9. In further examples the ScFv-Fc-TGFβtrap encoded by the HSV used in the methods is an anti-PD-L1 ScFv-Fc-TGFβtrap, and in some embodiments has a heavy chain variable region sequence having at least 95% identity to SEQ ID NO:20 and a light chain variable region sequence having at least 95% identity to SEQ ID NO:21. Further provided herein is a recombinant HSV for use in a method of treating cancer, where the method includes administering a recombinant HSV that comprises a nucleic acid construct encoding an ScFv-Fc-TGFβtrap protein to a subject having cancer. In some embodiments the cancer may be a solid tumor. The recombinant HSV can be any disclosed herein, such as, for example, any that encodes an ScFv-Fc-TGFβtrap that is able to bind an immune checkpoint inhibitor such as PD-1 or PD-L1. The recombinant HSV can in some embodiments further comprise at least one additional transgene, and may include an additional transgene encoding an immune activating cytokine, such as for example, IL12. The subject may be a human or may be a non-human animal such as, for example, a dog, cat, cow, bull, or horse. The cancer can be without limitation, bladder, bone, breast, eye, stomach, head and neck, kidney, liver, lung, ovarian, pancreatic, prostate, skin, or uterine cancer, a mesothelioma, a glioma, a neurocytoma, or a chondrosarcoma. The administering can be by any means and can be, as nonlimiting examples, parenteral, systemic, intracavitary (e.g, intrapleural, intraperitoneal), peritumoral, or intratumoral, and may be by injection, intravenous or intra-arterial infusion, or other delivery means. Injection can be, for example, parenteral, subcutaneous, intramuscular, intravenous, intra-arterial, intratumoral, or peritumoral. The treatment regimen may include more than one administration of the virus and can include multiple dosings over a period of days, weeks, or months.

In a further aspect, provided herein is an ScFv-Fc-TGFβRII_{ecto} fusion protein comprising a single chain variable fragment (ScFv) of an antibody that binds an immune checkpoint inhibitor, a TGFβRII ectodomain (TGFβRII_{ecto}), and an Fc antibody region linking the ScFv to the TGFβRII_{ecto}. The fusion protein can be an isolated, partially purified, or substantially purified protein. The ScFv of the fusion protein can specifically bind an immune checkpoint molecule such as PD-1 or PD-L1. In some embodiments the ScFv may be derived from a monoclonal antibody that binds PD-1 such as a BB9 monoclonal antibody, an RG1H10 monoclonal antibody, or pembrolizumab. For example, the ScFv moiety of the fusion protein can comprise an amino acid sequence having at least 95% identity to SEQ ID NO: 11, SEQ ID NO:15, or SEQ ID NO:19. In some embodiments an ScFv-Fc-TGFβRII_{ecto} fusion protein as provided herein comprises an scFv derived from the BB9 PD-1 antibody, and in some embodiments the scFv comprises a heavy chain variable region having CDRs of the sequences SEQ ID NO:63 (HC-CDR1), SEQ ID NO:64 (HC-CDR2), and SEQ ID NO:65 (HC-CDR3), and has a light chain variable region having CDRs of the sequences SEQ ID NO:66 (LC-CDR1), SEQ ID NO:67 (LC-CDR2), and SEQ ID NO:68 (LC-CDR3). The ScFv-Fc-TGFβRII_{ecto} fusion protein can include a heavy chain variable region having at least 95% identity to SEQ ID NO: and a light chain variable region having at least 95% identity to SEQ ID NO. In other embodiments the ScFv may be derived from a monoclonal antibody that binds PD-L1 such as a Combi5 monoclonal antibody, an H6B1LEM monoclonal antibody, or avelumab. For example, the ScFv moiety of the fusion protein can comprise an amino acid sequence having at least 95% identity to SEQ ID NO:23, SEQ ID NO:27, or SEQ ID NO:21. The TGFβRII_{ecto} moiety of the ScFv-Fc-TGFβRII_{ecto} fusion protein can in various embodiments be derived from human TGFβ receptor II, and can have at least 95% amino acid identity to SEQ ID NO:7. In some embodiments, the TGFβRII_{ecto} comprises SEQ ID NO:7. The Fc region that links the ScFv moiety of the ScFv-Fc-TGFβRII_{ecto} fusion protein to TGFβRII_{ecto} can be an Fc region of an IgG1 or can be an IgG4 Fc region. In exemplary embodiments the Fc region is a human IgG1 Fc region or a variant thereof (e.g., SEQ ID NO:5) or can be an IgG4 Fc region (SEQ ID NO:3) or a sequence having at least 95% amino acid identity thereto (e.g., SEQ ID NO:2). The fusion protein can optionally include a peptide linker between the Fc and TGFβRII_{ecto}.

Various embodiments of ScFv-Fc-TGFβRII_{ecto} fusion proteins as provided herein include ScFv-Fc-TGFβtrap fusion proteins in which the ScFv specifically binds PD-1 and is derived from monoclonal antibody BB9, monoclonal antibody RG1H10, or pembrolizumab and the Fc region is an IgG4 Fc region. For example, the anti-PD-1 ScFv-Fc-TGFβtrap fusion protein can have the sequence of SEQ ID NO:40, SEQ ID NO:42, or SEQ ID NO:44 or can have a sequence having at least 95% identity to any of SEQ ID NO:40, SEQ ID NO:42, or SEQ ID NO:44. Other examples of ScFv-Fc-TGFβtrap fusion proteins are ScFv-Fc-TGFβtraps in which the ScFv specifically binds PD-L1 and is derived from monoclonal antibody Combi5, monoclonal antibody H6B1LEM, or avelumab and the Fc region is an IgG4 Fc region. For example, the anti-PD-L1 ScFv-Fc-TGFβtrap fusion protein can have the sequence of SEQ ID NO:46, SEQ ID NO:48, or SEQ ID NO:50 or can have a sequence having at least 95% identity to any of SEQ ID NO:46, SEQ ID NO:48, or SEQ ID NO:50. In some embodiments an ScFv-Fc-TGFβRII_{ecto} fusion protein is provided in a virus-free conditioned medium, and in some embodiments an ScFv-Fc-TGFβRII_{ecto} fusion protein may be partially or substantially purified from a virus-free conditioned medium.

Also provided herein are conditioned media compositions, such as virus-free conditioned media (VFCM) compositions, comprising an ScFv-Fc-TGFβtrap fusion protein, such as any disclosed herein. The VFCM composition can be concentrated and formulated for use as a pharmaceutical composition. Further provided are pharmaceutical compositions that comprise an ScFv-Fc-TGFβtrap fusion protein as provided herein. In various embodiments, a VFCM composition includes, in addition to a ScFv-Fc-TGFβtrap fusion protein, an immune activating cytokine, such as, for example, IL12.

In another aspect provided herein is a method of treating cancer comprising administering a pharmaceutical composition that includes an ScFv-Fc-TGFβtrap fusion protein, such as any disclosed herein, to a subject having cancer. The method can include administering a recombinant HSV that comprises a nucleic acid construct encoding an ScFv-Fc-TGFβtrap that is able to bind an immune checkpoint inhibitor to a subject having cancer. The subject can be a human or can be a non-human animal such as a dog, cat, or horse. In some embodiments the cancer may be a solid tumor. The cancer can be, without limitation, bladder, bone, breast, eye, stomach, head and neck, kidney, liver, lung, ovarian, pancreatic, prostate, skin, or uterine cancer, a mesothelioma, a glioma, a neurocytoma, or a chondrosarcoma. The administering can be by any means and can be, as nonlimiting examples, parenteral, systemic, intracavitary (e.g, intrapleural, intrapulmonary, intraperitoneal), peritumoral, or intratumoral, and may be by injection, intravenous or intra-arterial infusion, or other delivery means. Injection can be, for example, parenteral, subcutaneous, intramuscular, intravenous, intra-arterial, intratumoral, or peritumoral. The treatment regimen may include more than one administration of the protein or protein composition and can include multiple dosing over a period of days, weeks, or months.

Yet another aspect of the disclosure is a nucleic acid construct comprising a sequence encoding any of the ScFv-Fc-TGFβtrap fusion proteins disclosed herein. The nucleic acid sequence encoding an ScFv-Fc-TGFβtrap fusion protein can be operably linked to a promoter, such as a eukaryotic promoter, such as a eukaryotic promoter operable in a mammalian cell. Nonlimiting examples of suitable promoters include the EF1α promoter, the HTLV promoter, the EF1α/HTLV fusion promoter, the CMV promoter, the JeT promoter, and functional derivatives thereof.

### BRIEF DESCRIPTION OF THE DRAWINGS

FIGS. 1A and 1B provide schematic diagrams of nucleic acid constructs that include engineered “ScFv-Fc-TGFβtrap” genes: FIG. 1A) ScFv-Fc-TGFβtrap gene construct operably linked to a EF1α/HTLV hybrid promoter (white arrow); solid black bar: signal peptide; left diagonal striped bar: ScFv of an antibody recognizing any of PD-1, PD-L1, or CTLA-4; white bar: human Fc region of IgG1 or IgG4; right diagonal striped bar: ectodomain of TGFβRII. FIG. 1B) dual gene construct including both an ScFv-Fc-TGFβtrap-encoding gene and a gene encoding IL12. The ScFv-Fc-TGFβtrap construct is the same as provided in FIG. 1A. The IL12 construct includes a CMV promoter operably linked to a single open reading frame that includes a sequence encoding the p40 subunit of IL12 followed by a sequence encoding a 2× elastin linker, followed by a sequence encoding the p35 subunit of IL12. Diagram is not to scale.

FIG. 2A provides a bar graph showing the results of MSD assays for TGFβRII content of VFCM harvested from A431 cell cultures infected with ScFv-Fc-TGFβtrap HSVs SepGI-097, three separate isolates of SepGI-137, and three separate isolates of SepGI-138, and FIG. 2B) provides a bar graph showing the results of MSD assays for TGFβRII content of VFCM harvested from HepG2 cell cultures infected with ScFv-Fc-TGFβtrap HSVs SepGI-097, three isolates of SepGI-137, and three isolates of SepGI-138. Negative controls for both graphs include VFCM from uninfected cell culture and VFCM from cell cultures infected with an HSV that does not express any exogenous transgene (SepGI-Null). n.d., not detected.

FIG. 3A provides the percentage of human CD8+ T cells expressing CD103 in response to TGFβ1 only (solid square), no TGFβ1 (solid triangle), or TGFβ1 plus a titration of recombinant human TGFβRII (solid circles); FIG. 3B provides a bar graph showing the percentage of human CD8+ T cells expressing CD103 in the presence of TGFβ31 and various VFCM from infected A431 cell cultures. VFCM lacking ScFv-Fc-TGFβtrap (black bars) include VFCM from uninfected cultures and SepGI-Null-infected cultures. VFCM containing ScFv-Fc-TGFβtrap include SepGI-097-, SepGI-137-, and SepGI-138-infected cultures; and FIG. 3C) provides a bar graph showing the percentage of human CD8+ T cells expressing CD103 in the presence of TGFβ1 and various VFCM from infected HepG2 cell cultures. VFCM lacking ScFv-Fc-TGFβtrap (black bars) include VFCM of uninfected and SepGI-Null-infected cultures. VFCM containing ScFv-Fc-TGFβtrap include VFCM of SepGI-097-, SepGI-137-, and SepGI-138-infected cultures.

FIG. 4A provides a standard curve titration of the anti-PD-1 IgG antibody BB9 in a luciferase assay responsive to blockade of the PD-1/PD-L1 interaction; FIG. 4B) provides a bar graph of the quantitation of PD-1 blocking activity in VFCMs of A431 cell cultures infected with ScFv-Fc-TGFβtrap HSVs SepGI-097, SepGI-137 (three isolates), and SepGI-138 (two isolates). Negative controls include VFCM from uninfected cell culture and VFCM from cell cultures infected with SepGI-Null, an HSV that does not express any exogenous transgene. n.d., not detected.

FIG. 5A provides a bar graph showing the results of MSD assays for TGFβRII content of VFCM harvested from uninfected A431 cell cultures and A431 cultures infected with SepGI-Null and SepGI-123 that do not include a ScFv-Fc-TGFβtrap transgene, as well as HSVs Sep GI-143, SepGI-144, and SepGI-145 that each include a ScFv-Fc-TGFβtrap transgene; and FIG. 5B) provides a bar graph showing the results of MSD assays for TGFβRII content of VFCM harvested from uninfected HepG2 cell cultures and HepG2 cell cultures infected with SepGI-Null and SepGI-123 that do not include a ScFv-Fc-TGFβtrap transgene, as well as HSVs Sep GI-143, SepGI-144, and SepGI-145 that each include a ScFv-Fc-TGFβtrap transgene. n.d., not detected.

FIG. 6A provides the percentage of human CD8+ T cells expressing CD103 in response to TGFβ1 only (solid square), no TGFβ1 (solid triangle), or TGFβ1 plus a titration of recombinant human TGFβRII (solid circles); FIG. 6B provides a bar graph showing the percent of human CD8+ T cells expressing CD103 in the presence of TGFβ1 and various VFCM from infected A431 cell cultures. VFCM lacking ScFv-Fc-TGFβtrap include VFCM from uninfected, SepGI-Null-infected, and SepGI-123-infected cultures. VFCM containing ScFv-Fc-TGFβtrap include VFCM from SepGI-143-infected, SepGI-144-infected, and SepGI-145-infected cultures; FIG. 6C) provides a bar graph showing the percent of human CD8+ T cells expressing CD103 in the presence of TGFβ1 and various VFCM from infected HepG2 cell cultures. VFCM lacking ScFv-Fc-TGFβtrap include VFCM from uninfected, SepGI-Null-infected, and SepGI-123-infected cultures. VFCM containing ScFv-Fc-TGFβtrap include VFCM from SepG1-143-infected, SepG1-144-infected, and SepGI-145-infected cultures.

FIG. 7A provides a standard curve titration of anti-PD-1 clone BB9 IgG in a luciferase assay responsive to blockade of the PD-1/PD-L1 interaction; FIG. 7B) provides a bar graph of the quantitation of PD-1 blocking activity in VFCMs of A431 cell cultures infected with ScFv-Fc-TGFβtrap HSVs SepG1-143, SepGI-144, and SepGI-145. Negative controls include VFCM from uninfected cell culture and VFCM from cell cultures infected with HSVs not expressing ScFv-Fc-TGFβtrap, SepGI-Null and SepGI-123. n.d., not detected.

FIG. 8A provides a standard curve titration of recombinant human IL-12 in a reporter cell-based luciferase assay responsive to IL-12 receptor binding and signaling; FIG. 8B provides a bar graph showing the IL12-responsive luminescence of reporter cells incubated with VFCM of A431 cells infected with the IL12 HSV SepGI-123, or ScFv-Fc-TGFβtrap+IL12 HSVs SepGI-143, SepGI-144, and SepGI-145; FIG. 8C provides a bar graph showing the IL12-responsive luminescence of reporter cells incubated with VFCM of HepG2 cells infected with the IL12 HSV SepGI-123, or ScFv-Fc-TGFβtrap+IL12 HSVs SepG1-143, SepGI-144, and SepGI-145. Negative controls for both graphs include VFCM from uninfected cell culture and VFCM from cell cultures infected with an HSV that does not express any exogenous transgene (SepGI-Null).

FIG. 9 provides a bar graph showing the results of MSD assays for TGFβRII content of VFCM harvested from A431 cell cultures infected with ScFv-Fc-TGFβtrap HSVs SepGI-143, SepGI-144, and four separate isolates of SepGI-158. Negative controls include VFCM from uninfected cell culture and VFCM from cell cultures infected with an HSV that does not express any exogenous transgene (SepGI-Null). n.d., not detected.

FIG. 10A provides a standard curve titration of anti-PD-1 clone BB9 IgG in a luciferase assay responsive to blockade of the PD-1/PD-L1 interaction and FIG. 10B provides a bar graph of the quantitation of PD-1 blocking activity in VFCMs of A431 cell cultures infected with ScFv-Fc-TGFβtrap HSVs SepGI-143, SepGI-145, and four isolates of SepGI-158. Negative controls include VFCM from uninfected cell culture and VFCM from cell cultures infected with an HSV that does not express any exogenous transgene (SepGI-Null). n.d., not detected.

FIG. 11A provides a standard curve titration of recombinant murine IL-12 in a reporter cell-based luciferase assay responsive to IL-12 receptor binding and signaling and FIG. 11B is a bar graph showing the concentration of IL12 (standardized to recombinant murine IL12) in VFCM of A431 cells infected with four different isolates of SepGI-162 HSV that expresses an RG1H10 anti-PD-1 cFv-Fc-TGFβtrap gene and the murine IL12 gene. Negative controls include VFCM from uninfected A431 cell culture and VFCM from A431 cell cultures infected with an HSV that does not express any exogenous transgene (SepGI-Null).

FIG. 12A) is a graph providing the total yield (pfu) of various viruses used to infect 3T6 cells, based on titers of supernatants obtained from 3T6 cells infected with HSV strain 17+(“Virttu 17+”), HSV1716 (Seprehvir®), SepGI-Null, and SepGI-145 removed one hour after infecting with virus or 72 hours post-infection, mock-infected supernatants were also titered as a control; FIG. 12B) provides the same data for infection of Vero cells with HSV strains HSV strain 17+(“Virttu 17+”), HSV1716 (Seprehvir®), SepGI-Null, and SepGI-145. FIG. 12C) is a bar graph showing the data of A) expressed as output/input virus pfu and FIG. 12D) is a bar graph showing the data of B) expressed as output/input virus pfu.

FIG. 13A) is a graph providing the total yield (pfu) of various viruses used to infect cKPF cells, based on titers of supernatants obtained from cKPF cells infected with HSV strain 17+(“Virttu 17+”), HSV1716 (Seprehvir®), SepGI-Null, and SepGI-145 removed one hour after infecting with virus or 72 hours post-infection, mock-infected supernatants were also titered as a control; FIG. 13B) provides the same data for infection of Vero cells with HSV strains HSV strain 17+(“Virttu 17+”), HSV1716 (Seprehvir®), SepGI-Null, and SepGI-145. FIG. 13C) is a bar graph showing the data of A) expressed as output/input virus pfu and FIG. 13D) is a bar graphing showing the data of B) expressed as output/input virus pfu.

FIGS. 14A-14C provide graphs of tumor volume over time of mice inoculated with MB49 bladder cancer cells on Day 0 and treated by peritumoral injection with formulation buffer, SepGI-Null HSV, or SepGI-162 on Day 8 and every other weekday thereafter for a total of nine treatments. FIG. 14A) is a graph of tumor volume over time of mice treated by injection with formulation buffer; FIG. 14B) is a graph of tumor volume over time of mice treated by injection with the SepGI-Null HSV (lacking exogenous transgenes); and FIG. 14C) is a graph of tumor volume over time of mice treated by injection with the SepGI-162 HSV that included a BB9 anti-PD-1 ScFv-Fc-TGFβtrap and the murine IL12 gene.

FIGS. 15A-15C provide graphs of the percent body weight change, relative to Day 0, of the mice represented in FIGS. 14A-14C. FIG. 15A) shows the percent body weight change over time of mice treated by injection with formulation buffer; FIG. 15B) shows the percent body weight change over time of mice treated by injection with the SepGI-Null HSV (lacking exogenous transgenes); and FIG. 15C) shows the percent body weight change over time of mice treated by injection with the SepGI-162 HSV that included a BB9 anti-PD-1 ScFv-Fc-TGFβtrap and the murine IL12 gene.

FIG. 16 provides a Kaplan Meier plot of percent survival over time of the tumor-implanted mice of FIGS. 14 and 15 treated with formulation buffer (solid line), SepGI-Null HSV lacking transgenes (dashed line), and SepGI-162 that included a BB9 anti-PD-1 ScFv-Fc-TGFβtrap and the murine IL12 gene (dotted line). Mice were euthanized when tumor volumes reached 2000 mm³.

FIGS. 17A-17C provide curves of tumor volume over time of mice re-challenged with a second inoculation of MB49 bladder cancer cells on the opposite flank to establish a secondary tumor after inoculation of a primary tumor and treatment with SepGI-162. Mice as shown in FIG. 14C that were inoculated with MB49 tumor cells on day −40 (day 0 in FIG. 14C) and demonstrated minimal tumor progression (one mouse) or elimination of the tumor (three mice, represented as a single line) were re-inoculated with tumor cells injected in the opposite flank on day 0 and both primary tumor growth at the original site and secondary tumor growth at the opposite flank site was monitored for an additional 3 weeks. FIG. 17A) is a graph showing tumor volume over time of control mice inoculated with tumor cells on day 0. Control mice had not received prior tumor inoculation or treatment. FIG. 17B) provides a graph of the growth of the original (primary) tumors in re-challenged mice for three weeks after secondary tumor inoculation on day 0. FIG. 17C) provides a graph of the growth of the secondary tumors in the re-challenged mice.

FIGS. 18A and 18B provide graphs of the percent change in body weight relative to day 0 of the rechallenge of control and secondary site tumor cell-inoculated mice of FIG. 17. FIG. 18A) percent change in body weights of control mice inoculated with tumor at day 0; FIG. 18B) percent change in body weight of mice that had received SepGI-162 as treatment of a primary tumor after inoculation with a secondary tumor on day 0.

FIG. 19 provides a plot of relative tumor weights of mice shown in FIGS. 17 and 18 that were inoculated with a secondary tumor on day 0 (FIG. 17B) after having been treated for a primary tumor with SepGI-162. Control mice were inoculated with tumor cells on day 0 but had not been inoculated with a primary tumor and were not treated with SepGI-162 (FIG. 17A). Tumor weights were obtained by dissection of tumors of euthanized mice; the tumor weights were divided by the number of days of tumor growth to provide relative tumor weights.

FIGS. 20A-20C provide proliferation curves of HSV-infected cells demonstrating the cytotoxicity of HSVs deleted in the R1 gene toward canine osteosarcoma OSCA-40 cells. OSCA-40 cells were seeded in 96-well E-plates (xCELLigence®, Roche) and infected at the indicated MOIs with FIG. 20A) SepGI-Null, FIG. 20B) SepGI-145, and FIG. 20C) SepGI-dsred. The proliferation curves of virus-infected cells and control uninfected cells were monitored in real time using the xCELLigence® real time cell analysis system. The uppermost curve represents uninfected cells; in order of descent the remaining curves represent cells infected at MOI 0.1, MOI 1, and MOI 10. Vertical lines indicate the time of infection. Cell index values (y axes) are proportional to cell numbers.

FIG. 21 provides a bar graph providing absorbance at 450 nm as the results of a sandwich ELISA to test for production and binding of TGFβRII_{ecto} fusion protein to canine TGFβR1 produced by SepGI-145-infected OSCA-40 cells. Results of VFCMs of untreated OSCA-40 cells, SepGI-Null-infected OSCA-40 cells, and SepGI-145-infected OSCA-40 cells (diluted 1:4) demonstrate that SepGI-145-infected OSCA-40 cells produce functional anti-PD-L1-Fc-TGFβRII_{ecto} fusion protein that binds canine TGFβ.

FIG. 22 provides the results of PD-1/PD-L1 Blockade Assay using concentrated VFCMs from OSCA-40 cells infected with SepGI-Null and SepGI-145 in Combi5 IgG equivalents.

FIG. 23A) provides a standard curve for recombinant IL12 for luminescence units. FIG. 23B) provides a graph showing the results of the IL12 assay using VFCMs from OSCA-40 cells infected with SepGI-Null and SepGI-145.

FIG. 24 is a bar graph providing the concentration of TGFβRII in concentrated virus-free conditioned media isolated from cultures of BHK cells infected with SepGI-Null HSV and ScFv-Fc-TGFβtrap HSVs SepGI-097, SepGI-138, SepGI-162, and SepGI-167.

FIG. 25A) is a graph showing the percent of human CD8+ T cells expressing CD103 in response to TGFβ1 only (solid square), no TGFβ1 (solid triangle), or TGFβ1 plus a titration of recombinant human TGFβRII (solid circles), and FIG. 25B) provides a bar graph showing the percent of human CD8+ T cells expressing CD103 in the presence of TGFβ1 and various concentrated VFCM from infected BHK cell cultures. Concentrated VFCM lacking ScFv-Fc-TGFβtrap is SepGI-Null. Concentrated VFCM containing ScFv-Fc-TGFβtrap includes SepGI-097, SepGI-138, SepGI162, and SepGI-167. Each concentrated VFCM was tested at 1:200, 1:400, 1:800, and 1:1600 dilutions.

FIG. 26 provides tumor volumes over time in mice inoculated with MB49 tumor cells and treated with the recombinant HSV SepGI-162. A) is a graph of tumor volume over time of 7 Group 1 mice inoculated with tumor cells and treated with formulation buffer 3 times per week for 3 weeks. B) is a graph of tumor volume over time of 7 Group 2 mice inoculated with tumor cells and beginning 8 days later treated with the SepGI-Null control HSV 3 times per week for 3 weeks. C) is a graph of tumor volume over time of 8 Group 3 mice inoculated with tumor cells and beginning 8 days later treated with the SepGI-162 recombinant HSV encoding an anti-PD-1 scFv-Fc-TGFβTrap and IL12 3 times per week for 3 weeks. D) is a graph of tumor volume over time of 8 Group 4 mice inoculated with tumor cells and treated with the SepGI-162 recombinant HSV encoding an anti-PD-1 scFv-Fc-TGFβTrap and IL12 3 times over two weeks. E) is a graph of tumor volume over time of 8 Group 4 mice inoculated with tumor cells and treated with the SepGI-162 recombinant HSV encoding an anti-PD-1 scFv-Fc-TGFβTrap and IL12 3 times over one week. F) is a graph of tumor is a graph of tumor volume over time of 8 Group 4 mice inoculated with tumor cells and treated with the SepGI-162 recombinant HSV encoding an anti-PD-1 scFv-Fc-TGFβTrap and IL12 once per week for one week.

FIG. 27 provides tumor volumes over time in mice inoculated with MB49 tumor cells and treated with the recombinant HSV SepGI-167. A) is a graph of tumor volume over time of 6 Group 1 mice inoculated with tumor cells and treated with formulation buffer 3 times per week for 2 weeks. B) is a graph of tumor volume over time of 6 Group 2 mice inoculated with tumor cells and treated with 1×10⁷ pfu of recombinant HSV SepGI-167 encoding an anti-PD-L1 scFv-Fc-TGFβTrap and IL12 3 times per week for 2 weeks. C) is a graph of tumor volume over time of 6 Group 3 mice inoculated with tumor cells and treated with 1×10⁶ pfu SepGI-167 3 times per week for 2 weeks. D) is a graph of tumor volume over time of 6 Group 4 mice inoculated with tumor cells and treated with 1×10⁵ pfu SepGI-167 3 times per week for 2 weeks. E) is a graph of tumor volume over time of 6 Group 5 mice inoculated with tumor cells and treated with 1×10⁷ pfu of recombinant HSV SepGI-167 3 times over the course of 1 week. F) is a graph of tumor volume over time of 6 Group 6 mice inoculated with tumor cells and treated with 1×10⁶ pfu of recombinant HSV SepGI-167 3 times over the course of 1 week. G) is a graph of tumor volume over time of 6 Group 7 mice inoculated with tumor cells and treated with 1×10⁵ pfu of recombinant HSV SepGI-167 3 times over the course of 1 week.

### DETAILED DESCRIPTION OF THE INVENTION

### Definitions

Technical and scientific terms used herein have meanings that are commonly understood by those of ordinary skill in the art unless defined otherwise. Generally, terminologies pertaining to techniques of virology, cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are also well known and commonly used in the art. Standard techniques can be used for chemical syntheses, chemical analyses, pharmaceutical preparation, formulation, and delivery, and treatment of patients.

All of the references cited herein are incorporated herein by reference in their entireties.

Headings provided herein are solely for the convenience of the reader, and do not limit the various aspects of the disclosure, which can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited to a single entity.

The use of the alternative (e.g., “or”) is to be understood to mean either one or both or any combination thereof of the alternatives and the term “and/or” means specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following: A, B, and C; A, B, or C; A or C; A or B; B or C; A and C; A and B; B and C; A (alone); B (alone); and C (alone).

As used herein, terms “comprising”, “including”, “having” and “containing” and their grammatical variants are intended to be non-limiting so that the item referred to or multiple items listed do not exclude other items that can be added to the listed item(s).

The term “consisting essentially of” means that the composition, method or structure may include additional ingredients, steps and/or parts that do not materially alter the basic and novel characteristics of the claimed composition, method or structure.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. When particular values or compositions are provided in the instant disclosure, unless otherwise stated, the meaning of “about” or “approximately” should be assumed to be within an acceptable error range for that particular value or composition. When ranges for values are provided it is intended that values include the boundaries of the ranges.

The terms “peptide”, “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides include precursor molecules and mature molecules.

The terms “nucleic acid” “nucleic acid molecule”, “nucleic acid (or DNA or RNA) fragment”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (for example cDNA or genomic DNA), RNA molecules (e.g., mRNA), analogs of the DNA or RNA generated using nucleotide analogs (e.g., peptide nucleic acids, locked nucleic acids, and nucleic acids or nucleic acid analogs having one or more non-naturally occurring nucleotide analogs), and hybrids thereof. Nucleic acid molecules can be single-stranded or double-stranded. Although “base pair(s)” or “bp” is typically used to refer to the length of a double-stranded nucleic acid molecule it may in some instances be used interchangeably with nucleotide(s) (nt), where either may be used to refer to the length of a single-stranded or double-stranded nucleic acid molecule.

The term “gene” is used broadly to refer to any segment of a nucleic acid molecule (typically DNA, but optionally RNA) encoding a polypeptide or expressed RNA. Thus, genes include sequences encoding expressed RNA (which can include polypeptide coding sequences or, for example, functional RNAs, such as ribosomal RNAs, tRNAs, antisense RNAs, microRNAs, short hairpin RNAs, ribozymes, etc.). A gene may optionally further comprise regulatory sequences required for or affecting the expression of sequences linked to the regulatory sequences. Gene may also encompass sequences associated with the protein or RNA-encoding sequence in its natural state, such as, for example, intron sequences, 5′ or 3′ untranslated sequences, etc. In some instances, a gene may only refer to a protein-encoding portion of a DNA or RNA molecule, which may or may not include introns. A gene is generally greater than 50 nucleotides in length, such as greater than 100 nucleotide in length, and can be, for example, between 50 nucleotides and 500,000 nucleotides in length, such as between 100 nucleotides and 100,000 nucleotides in length or between about 200 nucleotides and about 50,000 nucleotides in length, or about 200 nucleotides and about 20,000 nucleotides in length. Genes can be obtained from a variety of sources, including but not limited to cloning from a source or sources of interest or synthesizing from known or predicted sequence information.

A nucleic acid sequence, nucleic acid molecule, or gene may be “derived from” an indicated source, which can include the isolation (in whole or in part) of a nucleic acid segment from an indicated source. A nucleic acid molecule may also be derived from an indicated source by, for example, direct cloning, PCR amplification, or DNA synthesis from the indicated polynucleotide source or based on a sequence associated with the indicated polynucleotide source (e.g., a sequence in a database, a published sequence, a sequence determined by DNA sequencing). Genes or nucleic acid molecules derived from a particular source or species also include genes or nucleic acid molecules having sequence modifications with respect to the source nucleic acid molecules. For example, a gene or nucleic acid molecule derived from a source (e.g., a particular referenced gene) can include one or more mutations with respect to the source gene or nucleic acid molecule that are unintended or that are deliberately introduced, and if one or more mutations, including substitutions, deletions, or insertions, are deliberately introduced the sequence alterations can be introduced by any means, including random or targeted mutation of cells or nucleic acids, amplification or other molecular biology synthesis techniques, or by chemical synthesis, or any combination thereof. A gene or nucleic acid molecule that is derived from a referenced gene or nucleic acid molecule that encodes a functional RNA or polypeptide can encode a functional RNA or polypeptide having at least 75%, at least 80%, at least 85%, at least 90%, or at least 95%, sequence identity with the referenced or source functional RNA or polypeptide, or to a functional fragment thereof. For example, a gene or nucleic acid molecule that is derived from a referenced gene or nucleic acid molecule that encodes a functional RNA or polypeptide can encode a functional RNA or polypeptide having at least 85%, at least 90%, and can have at least 95%, at least 96%, at least 97%, at least 98%, or at least 99% sequence identity with the referenced or source functional RNA or polypeptide, or to a functional fragment thereof.

The term “derivative” is used herein to refer to a nucleic acid molecule (or polypeptide derived from the referenced a nucleic acid molecule), viral genome, virus, viral genome, virus, or polypeptide by an alteration of the nucleotide or amino acid sequence. For example, sequence variants that have, for example, at least 85%, at least 90% or at least 95% nucleotide or amino acid sequence may be referred to as derived from the referenced nucleic acid molecule or polypeptide. Codon-optimized nucleic acid sequences are also “derived from” the non-codon optimized sequence from which they are designed. Polypeptides that include amino acid sequence insertions, including functional domains, such as but not limited to protein tags for identification or purification, signal, leader, transit peptide, or nuclear localization sequences, SUMO sequences, and the like, are considered “derived from” the original polypeptide that does not include the insertion. Similarly, proteins having deleted sequences, including deleted functional domains, may be referred to as “derived from” the original protein that includes the domain.

As used herein, the term “derivative” in reference to a polypeptide can also refer to a polypeptide that has been chemically modified, e.g., via conjugation to another chemical moiety such as, for example, polyethylene glycol, albumin (e.g., human serum albumin), or post-translationally modified such as by phosphorylation or glycosylation.

As used herein, the term “variant” polypeptides and “variants” of polypeptides refers to polypeptides comprising an amino acid sequence with one or more amino acid residues inserted into, deleted from and/or substituted into the amino acid sequence relative to a reference polypeptide sequence. For example, polypeptide sequence variants may have at least 85%, at least 90% or at least 95% amino acid sequence identity with the referenced nucleic acid molecule or polypeptide. In the same manner, a variant polynucleotide comprises a nucleotide sequence with one or more nucleotides inserted into, deleted from and/or substituted into the nucleotide sequence relative to another polynucleotide sequence. Preferably a protein variant has substantially the same activity or function as the protein from which it is derived. For example, a variant ScFv as provided herein having at least 95% amino acid identity to a disclosed ScFv can have substantially the same binding specificity and binding affinity for the antigen as the referenced ScFv.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software implementing a suitable algorithm such as the local homology algorithm of Smith and Waterman (Add. APL. Math. 2:482, 1981), by the global homology alignment algorithm of Needleman and Wunsch (J. Mol. Biol. 48:443, 1970). Those skilled in the art can determine appropriate parameters for aligning sequences, including any algorithms needed to achieve maximal alignment over the full length of the sequences being compared. “Percentage of sequence identity” or “percent (%) [sequence] identity,” as used herein, is determined by comparing two optimally locally aligned sequences over a comparison window defined by the length of the local alignment between the two sequences. (This may also be considered percentage of homology or “percent (%) homology”.) The amino acid sequence in the comparison window may comprise additions or deletions (e.g., gaps or overhangs) as compared to the reference sequence for optimal alignment of the two sequences. Local alignment between two sequences only includes segments of each sequence that are deemed to be sufficiently similar according to a criterion that depends on the algorithm used to perform the alignment. The percentage identity is calculated by determining the number of positions at which the identical nucleic acid base or amino acid residue occurs in both sequences to yield the number of matched positions, dividing the number of matched positions by the total number of positions in the window of comparison and multiplying the result by 100. GAP and BESTFIT, for example, can be employed to determine the optimal alignment of two sequences that have been identified for comparison. Typically, the default values of 5.00 for gap weight and 0.30 for gap weight length are used.

Similarities between polypeptides having the same or similar function can be at least 95%, or at or at least 96% identical, or at least 97% identical, or at least 98% identical, or at least 99% identical. In some examples, the amino acid substitutions can comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The inclusion of relatively short amino acid sequences (e.g., 60 amino acids or fewer, preferably 40 amino acids or fewer or 24 amino acids or fewer) in a polypeptide that encode functional domains for localization, detection, purification, or attachment of the polypeptide to other moieties (such as peptide linkers, amino acid tags, signal peptides, nuclear localization sequences, and the like) that do not substantially affect the essential function or activity of the polypeptide, are not taken into account in determining percent identity of the polypeptide to another polypeptide sequence.

An “endogenous” nucleic acid molecule, gene or protein is a native nucleic acid molecule, gene or protein as it occurs in, or is naturally produced by, the host.

“Exogenous nucleic acid molecule” or “exogenous gene” (also referred to herein as a “transgene”) refers to a nucleic acid molecule or gene that has been introduced (“transformed”) into a cell or introduced into a genome, such as a viral genome. A transformed cell may be referred to as a recombinant cell, into which additional exogenous gene(s) may be introduced. A descendent of a cell transformed with a nucleic acid molecule is also referred to as “transformed” if it has inherited the exogenous nucleic acid molecule. Similarly, a recombinant or engineered virus is a virus into which an exogenous nucleic acid molecule has been introduced. An exogenous nucleic acid molecule or construct or gene introduced into a virus is typically by insertion of the exogenous nucleic acid molecule, construct, or gene into the viral genome.

The terms “genetically engineered” “engineered” and “recombinant” are used interchangeably to refer to organisms, viruses, vectors, and constructs that have been made by human intervention using molecular cloning techniques which can include, but are not limited to, chemical synthesis of nucleic acid molecules, nucleic acid molecule synthesis by isolated polymerases or reverse transcriptases, restriction of DNA, ligation of DNA, polymerase chain reaction (PCR), in vitro or in vivo DNA editing (restriction, site directed mutation, and/or gene insertion) using CRISPR systems and/or cas enzymes, or in vitro or in vivo site-specific recombination.

The term “recombinant protein” as used herein refers to a protein produced by genetic engineering, e.g., by genetic engineering of a virus or cell to include a gene or nucleic acid construct encoding the protein.

When applied to organisms or viruses, the term recombinant, engineered, or genetically engineered refers to organisms or viruses that have been manipulated by introduction of a heterologous or exogenous recombinant nucleic acid sequence into the organism or virus or its genome, and includes gene knockouts, targeted mutations, and gene replacement, promoter replacement, deletion, or insertion, as well as introduction of transgenes or synthetic genes into the organism or virus or its genome. Recombinant or genetically engineered organisms or viruses can also be organisms into which constructs for gene “knock down” have been introduced. Such constructs include, but are not limited to, RNAi, microRNA, shRNA, siRNA, antisense, and ribozyme constructs. Also included are organisms or viruses whose genomes have been altered by the activity of meganucleases, zinc finger nucleases, or cas enzymes of CRISPR systems. An exogenous or recombinant nucleic acid molecule can be integrated into the genome of a recombinant/genetically engineered organism or virus or in other instances are not integrated into the genome of the recombinant/genetically engineered organism's genome. As used herein, “recombinant (micro)organism” or “recombinant host cell” or “recombinant virus” includes progeny or derivatives of the recombinant organism, cell, or virus. Because certain modifications may occur in succeeding generations due to either mutation or environmental influences, such progeny or derivatives may not, in fact, be identical to the parent cell, but are still included within the scope of the term as used herein.

The term “promoter” refers to a nucleic acid sequence capable of binding RNA polymerase in a cell and initiating transcription of a downstream (3′ direction) coding sequence. A promoter includes the minimum number of bases or elements necessary to initiate transcription at levels detectable above background. A promoter can include a transcription initiation site as well as protein binding domains (consensus sequences) responsible for the binding of RNA polymerase. Eukaryotic promoters often, but not always, contain “TATA” boxes and “CAT” boxes. Prokaryotic promoters may contain −10 and −35 prokaryotic promoter consensus sequences. A large number of promoters, including constitutive, inducible and repressible promoters, from a variety of different sources are well known in the art. Representative sources include for example, algal, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (initiate transcription in one direction) or bi-directional (initiate transcription in either direction). A promoter may be a constitutive promoter, a repressible promoter, or an inducible promoter.

A gene is “operably linked” to a regulatory sequence (such as a promoter) when the regulatory sequence affects the expression (e.g., the level, timing, or location of expression) of the gene.

As used herein “attenuated” means reduced in amount, degree, intensity, or strength. Attenuated gene expression may refer to a significantly reduced amount and/or rate of transcription of the gene in question, or of translation, folding, or assembly of the encoded protein. As nonlimiting examples, an attenuated gene may be a mutated or disrupted gene (e.g., a gene disrupted by partial or total deletion, truncation, frameshifting, or insertional mutation) or having decreased expression due to alteration of gene regulatory sequences.

The term host cell may be used herein to refer to a cell infected with a virus, such as an oncolytic virus, such as herpes simplex virus (HSV). In many cases, host cells as disclosed herein are infected with a recombinant HSV. Nonlimiting examples of host cells for the propagation of virus or production of virus-encoded recombinant protein include, without limitation, Vero cells, BHK cells, A431 cells, MB49 cells, and HepG2 cells. Preferably a host cell is productively infected with a virus such as HSV, that is, the host cell propagates the virus, i.e., when infected can produce infectious virus. A host cell as disclosed herein will be a eukaryotic cell, for example, a mammalian cell (e.g., a human cell, a monkey cell, a hamster cell, a rat cell, a canine cell, an equine cell, a mouse cell). Notably, various host cells are disclosed herein that are infected by recombinant viruses in which the host cells, when cultured under appropriate conditions or delivered into a subject, produce proteins encoded by genes engineered into the recombinant viruses. Such virally-encoded recombinant proteins may be secreted by the host cells into the culture medium (for cultured host cells) or extracellular milieu (for subject-administered host cells). In one example, the polypeptides are produced by recombinant nucleic acid methods by inserting a nucleic acid sequence (e.g., DNA) encoding the polypeptide into a viral genome. The virus is used to infect a host cell and the exogenous nucleic acid sequence is expressed by the host cell under conditions promoting expression, which may be in vivo conditions.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties.

As used herein, an “isolated” nucleic acid or protein is removed from its natural milieu or the context in which the nucleic acid or protein exists in nature. For example, an isolated protein or nucleic acid molecule is removed from the cell or organism with which it is associated in its native or natural environment. An isolated nucleic acid or protein can be, in some instances, partially or substantially purified, but no particular level of purification is required for isolation. Thus, for example, an isolated nucleic acid molecule can be a nucleic acid sequence that has been excised from the chromosome, genome, or episome that it is integrated into in nature.

A “purified” nucleic acid molecule or nucleotide sequence, or protein or polypeptide sequence, is substantially free of cellular material and cellular components. The purified nucleic acid molecule or protein may be free of chemicals beyond buffer or solvent, for example. “Substantially free” is not intended to mean that other components beyond the novel nucleic acid molecules are undetectable.

The terms “naturally-occurring” and “wild type” refer to a form found in nature. For example, a naturally occurring or wild type nucleic acid molecule, nucleotide sequence or protein may be present in and isolated from a natural source and is not intentionally modified by human manipulation.

In certain embodiments, the antibodies and fusion proteins described herein (e.g., ScFv-Fc-TGFβtrap proteins) can further comprise post-translational modifications. Exemplary post-translational protein modifications include glycosylation, phosphorylation, acetylation, methylation, ADP-ribosylation, ubiquitination, afucosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, glycosylation can be sialylation, which conjugates one or more sialic acid moieties to the polypeptide. Sialic acid moieties improve solubility and serum half-life while also reducing the possible immunogenicity of the protein. See Raju et al. (2001) Biochemistry 40:8868-76.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” (about 50-70 kDa) chain. The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989), incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005); AHo (Honegger and Pluckthun, J Mol Biol 309(3):657-670; 2001); Chothia (Al-Lazikani et al., 1997 J Mol Biol 273:927-948; and Contact (Maccallum et al., 1996 J Mol Biol 262:732-745.

An “antibody” and “antibodies” and related terms used herein refers to an intact immunoglobulin or to an antigen binding portion thereof that binds specifically to an antigen. Unless otherwise indicated, the term “antibody” includes, in addition to antibodies comprising full-length heavy chains and full-length light chains, derivatives, variants, fragments, and muteins thereof, examples of which are disclosed below. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, nanobodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv) antibodies, camelized antibodies, affibodies, disulfide-linked Fvs (sdFv), anti-idiotypic antibodies (anti-Id), minibodies, and nanobodies. Antibodies include monoclonal and polyclonal populations.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). Antibodies described herein that are referred to by the name of their antigen (e.g., “an antibody to PD-1”, “an anti-PD-1 antibody”, or “a PD-1 antibody”) are antibodies that specifically bind the referenced antigen. In various embodiments, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. The fusion proteins described herein include an ScFv that specifically binds an immune checkpoint molecule along with other protein domains. Binding specificity of an antibody or antigen binding protein or antibody fragment can be measured for example by ELISA, radioimmune assay (RIA), MSD assay, immunoradiometric assay (IRMA), electrochemiluminescence assays (ECL), or enzyme immune assay (EIA). A dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; and Fd fragments, as well as dAb; diabodies; linear antibodies; and polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; and Ward et al., Nature 341:544-546, 1989).

A “single-chain variable fragment” or “ScFv” is a fusion protein of the variable regions of the heavy (VH) and light (VL) chains of immunoglobulins, connected with a short linker peptide of approximately ten to 25 amino acids. The linker is usually rich in glycine for flexibility, and includes one or more serine or threonine residues for solubility, and can either connect the N-terminus of the VH with the C-terminus of the VL, or vice versa. An scFv lacks the constant regions of the antibody from which it is derived, and includes a linker connecting the heavy chain and light chain variable regions so that the antibody is a single chain only, but is designed to retain the specificity of the original immunoglobulin.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions and may or may not include a portion of the hinge region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In one embodiment, the Fc region can include a mutation that increases or decreases any one or any combination of these functions. In one embodiment, the Fc domain comprises a LALA-PG mutation (L234A, L235A, P329G) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex. Fc regions can also dimerize through interpeptide disulfide linkages. Thus fusion proteins that include an IgG1 or IgG4 Fc can form homodimers. In some embodiments for IgG4 Fc domains, interpeptide disulfide linkages to form homodimers can be favored over intrapeptide disulfide linkages that do not dimerize through a S228P mutation in the hinge region.

The present disclosure provides therapeutic compositions comprising any of the recombinant HSVs that are described herein, or recombinant protein compositions described herein in an admixture with a pharmaceutically-acceptable carrier or excipient. Excipients encompass, for example, carriers, stabilizers, diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers. Where a therapeutic composition comprises cells, the pharmaceutically-acceptable excipients will be chosen so as not to interfere with the viability or activity of the cells.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation.

The terms “effective amount”, “therapeutically effective amount” or “effective dose” or related terms may be used interchangeably and refer to an amount of virus or polypeptide as described herein that when administered to a subject, is sufficient to effect a measurable improvement or prevention of a disease or disorder associated. Therapeutically effective amounts will vary depending upon the relative activity of the viruses or polypeptides (e.g., in inhibiting tumor growth), the weight and age and sex of the subject, the severity of the disease condition in the subject, the manner of administration, and the like, which can be determined by one of ordinary skill in the art.

### Recombinant Oncolytic Viruses

Oncolytic viruses provide a targeted approach to cancer therapy, as they selectively replicate in and lyse tumor cells. Various types of oncolytic viruses are known in the art, including include parvoviruses, myxoma virus, Reovirus, Newcastle disease virus (NDV), Seneca Valley virus (SVV), poliovirus (PV), measles virus (MV), vaccinia virus (VACV), adenovirus, vesicular stomatitis virus (VSV), and herpes simplex virus (HSV). These viruses replicate in tumor cells and cause cell lysis and/or induce an immune response to the tumor cells they infect. This disclosure provides recombinant oncolytic viruses that include a heterologous gene construct that encodes an ScFv-Fc-TGFβtrap construct as disclosed herein. The construct can include a promoter active in a mammalian cell operably linked to the ScFv-Fc-TGFβtrap-encoding sequence and the construct can be inserted into the genome of the oncolytic virus.

In various embodiments, an oncolytic virus modified for expression of a ScFv-Fc-TGFβtrap can be a herpes simplex virus (Human alphaherpesvirus; HSV), such as an HSV-1, HSV-2, or a recombinant HSV having sequences of both HSV-1 or HSV-2. For example, a laboratory strain or clinical isolate of an HSV-1 or HSV-2 strain can be used. Multiple isolated and modified strains of HSV-1 and HSV-2 are known in the art and can be considered for use in the compositions and methods disclosed herein, including, as nonlimiting examples, HSV-1 strain A44, HSV-1 strain Angelotti, HSV-1 strain CL101, HSV-1 strain CVG-2, HSV-1 strain H129, HSV-1 strain HFEM, HSV-1 strain HZT, HSV-1 strain JS1, HSV-1 strain MGH10, HSV-1 strain MP, HSV-1 strain Patton, HSV-1 strain R15, HSV-1 strain R19, HSV-1 strain RH2, HSV-1 strain SC16, HSV-1 strain KOS, HSV-1 strain F, and HSV-1 strain 17, HSV-2 strain 186, HSV-2 strain 333, HSV-2 strain B4327UR, HSV-2 strain G, HSV-2 strain G, HSV-2 strain HG52, HSV-2 strain SA8, HSV-2 strain SD90, HSV-2 strain SN03, HSV-2 strain SS01, and HSV-2 strain ST04. Also considered for use in the compositions and methods provided herein are derivates or mutants of these strains or others that may be known in the art or isolated.

Derivatives of viral strains include, without limitation, viruses that may have one or more endogenous genes that is mutated, including one or more endogenous genes that is partially or entirely deleted, may have a transgene (heterologous gene) inserted into the viral genome (including but not limited to one or more selectable markers, negative selectable markers (“suicide genes”), and/or detectable markers (e.g., a gene encoding a fluorescent protein or a gene encoding an enzyme that produces a detectable product)), and/or may have one or more modifications such as but not limited to restriction sites, recombination sites or “landing pads”, exogenous promoters, etc. A derivative may have other modifications such as but not limited to deletion or mutation of non-gene sequences, such as for example gene regulatory regions such as promoters or non-coding sequences such as but not limited to direct or inverted repeat sequences. Derivatives of viral strains may be viruses that alternatively or in addition to other modifications include one or more transgenes supporting or regulating viral growth or viability, one or more genes affecting host cell functions, or one or more transgenes encoding therapeutic proteins, as nonlimiting examples.

In some nonlimiting embodiments the HSV is an HSV-1 such as HSV-1 strain 17, HSV-1 strain KOS, or HSV-1 strain F, or a derivative of any of HSV-1 strain 17, HSV-1 strain KOS, or HSV-1 strain F. For example, a strain used for the introduction of an ScFv-Fc-TGFβtrap construct can be HSV-1 strain 17 mutant 1716, HSV-1 strain F mutant R3616 (Chou & Roizman (1992) Proc. Natl. Acad. Sci. 89: 3266-3270), HSV-1 strain F mutant G207 (Toda et al. (1995) Human Gene Therapy 9:2177-2185), HSV-1 strain F mutant G47A (Todo et al. (2001) Proc Natl Acad Sci USA 98:6396-6401), HSV-1 mutant NV1020 (Geevarghese et al. (2010) Human Gene Therapy 21:1119-28), RE6 (Thompson et al. (1983) Virology 131:171-179), KeM34.5 (Manservigi et al. (2010) The Open Virology Journal 4:123-156), M032 (Campadelli-Fiume et al. (2011) Rev Med. Virol 21:213-226), Baco (Fu et al. (2011) Int. J. Cancer 129:1503-10), M032 or C134 (Cassady et al. (2010) The Open Virology Journal 4:103-108), or Talimogene laherparepvec (“TVec”, formerly OncoVex®; Liu et al. (2003) Gene Therapy 10:292-303), or a further derivative or mutant of any of these.

Mutation of endogenous viral genes can include mutation or deletion of genes that affect replication or propagation of the virus in non-cancerous cells or the ability of viruses to avoid host defenses. For example, an HSV that includes a ScFv-Fc-TGFβtrap construct can be deleted in any of the ICP34.5-encoding gene, the ICP6-encoding gene, the ICPO-encoding gene, the vhs-encoding gene, or the ICP27-encoding gene. Mutants that do not produce a functional protein encoded by a gene or genes (where the gene is multicopy) are referred to herein as having a functionally deleted gene. Functional deletion of one or more of the ICP34.5-encoding gene, the ICP6-encoding gene, the ICPO-encoding gene, and the vhs-encoding gene can result in an HSV impaired in replication in noncancerous cells.

The ICP34.5-encoding gene RL1 is located in the long repeat (RL) of the HSV-1 genome and is present in two copies. In some embodiments one or both copies of the ICP34.5-encoding genes is mutated or is partially or entirely deleted such that no functional protein is made. In preferred embodiments, an oncolytic HSV that includes a transgene encoding an ScFv-Fc-TGFβtrap protein and, optionally, an IL12 gene, is functionally deleted for the ICP34.5-encoding gene responsible for neurovirulence (Chou et al. (1990) Science 250:1262-1266), e.g., both copies of the ICP34.5-encoding gene of the HSV viral genome are inactivated. For example the oncolytic HSV used for introduction of an ScFv-Fc-TGFβtrap construct can be a mutant of HSV-1 strain 17 and may be HSV1716 (Brown et al. (1994) Journal of General Virology 75: 2367-2377; MacLean et al. (1991) Journal of General Virology 72:631-639) or a mutant or derivative thereof, or may be Seprehvec™ or a derivative or mutant thereof. HSV1716 and Seprehvec™ both have deletions in both copies of the RL1 gene such that they do not produce a functional gene product, but each otherwise has a genome substantially similar to that of HSV strain 17, which has been completely sequenced (Pfaff et al. (2016) J Gen Virol 97:2732-2741; ncbi.nlm.nih.gov/genome, Accession number JN555585). HSV1716 as a 755 bp deletion at the RL1 gene loci, whereas the Seprehvec RL1 deletion is 695 bp, beginning upstream of the coding region of the gene and extending through most of the coding region. These deleted regions can serve as sites for insertion of the transgenes described herein.

The

Recombinant HSVs as provided herein can have one or more transgenes inserted into the ICP34.5locus, the ICP6 locus, the ICPO locus, or the vhs locus. In some preferred embodiments a recombinant oncolytic HSV as provided herein can have one or both of an ScFv-Fc-TGFβtrap protein gene and an IL12 gene inserted into a deleted ICP34.5-encoding gene locus. In some preferred embodiments a recombinant oncolytic HSV as provided herein is functionally deleted for ICP34.5 (i.e., is ICP34.5 null) and has an ScFv-Fc-TGFβtrap protein gene and an IL12 gene inserted into both copies of the ICP34.5-encoding gene locus.

The recombinant oncolytic viruses provided herein include expression constructs that encode novel fusion proteins that simultaneously disrupt the interactions of an immune checkpoint protein and of TGFβ (ScFv-Fc-TGFβtrap fusion protein constructs) that comprise a single polypeptide chain that can be expressed and secreted by cells infected by the recombinant viruses that encode them. The ScFv moiety of the ScFv-Fc-TGFβtrap protein specifically binds an immune checkpoint protein and the TGFβtrap moiety binds and sequesters TGFβ. The ScFv moiety of the fusion protein is linked to the ectodomain of a TGFβ receptor II (TGFβRII_{ecto}) via an antibody Fc region, for example an IgG1 Fc region (e.g., SEQ ID NO:5 or a variant having at least 95% identity thereto) or an IgG4 Fc region (e.g., SEQ ID NO:3 or a variant having at least 95% identity thereto such as SEQ ID NO:2).

The ScFv-Fc-TGFβtrap fusion proteins encoded by expression constructs of recombinant HSVs as provided herein can include an ScFv antibody that binds the programmed cell death protein-1 (PD-1) or programmed death ligand 1 (PD-L1). PD-1 is a type I membrane protein that is a member of the extended CD28/CTLA-4 family of T cell regulators expressed on activated macrophages and T cells. PD-L1, a ligand of PD-1, is a 40 kDa type 1 transmembrane protein expressed on antigen-presenting cells such as activated monocytes and dendritic cells as well as on many tumor cells. Binding of PD-1, which is expressed on activated T cells, including tumor infiltrating T cells, to PD-L1, which is upregulated on many tumors, can suppress the activation (proliferation and cytokine production) of PD-1 expressing T lymphocytes (Topalian et al. (2012) Curr. Opin. Immunol. 24:207-212).

The ScFv of an ScFv-Fc-TGFβtrap proteins as provided herein can be derived from a monoclonal antibody that specifically binds PD-1 or PD-L1, that is, can have the heavy chain variable and light chain variable sequences of a monoclonal antibody that specifically binds PD-1 or PD-L1. The heavy chain variable and light chain variable antibody regions are joined by a peptide linker. For example, a peptide linker connecting the heavy chain variable region and light chain variable region can be of from eight to thirty amino acids in length, such as from about ten to about twenty-five amino acids in length, where the linker preferably includes multiple glycine residues to provide flexibility and includes one or more hydroxylated amino acids (e.g., serine or threonine) for solubility. One example of a suitable linker is a (GGGGS)n linker (SEQ ID NO: 61), where n can be, for example, between 1 and 30, between 1 and 24, between 1 and 12, or between 1 and 6, such as for example the linker (GGGGS)₃ of SEQ ID NO:56.

The ScFv moiety of an ScFv-Fc-TGFβtrap protein can include the variable region of the heavy chain of an antibody to PD-1 attached via a peptide linker to variable region of the light chain of the antibody to PD-1 or can include the variable region of the heavy chain of an antibody to PD-L1 attached via a peptide linker to variable region of the light chain of the antibody to PD-L1. As demonstrated herein, cells infected with recombinant viruses encoding anti-PD-1 ScFv-Fc-TGFβtrap proteins or anti-PD-L1 ScFv-Fc-TGFβtrap proteins produce and secrete functional anti-PD-1 ScFv-Fc-TGFβtrap or anti-PD-L1 ScFv-Fc-TGFβtrap proteins that are able to disrupt both PD-1/PD-L1 signaling pathways and TGFβ signaling pathways. In some embodiments the ScFv moiety of the fusion protein encoded by an expression construct of a recombinant HSV can be derived from a monoclonal antibody that specifically binds PD-1 such as the BB9 anti-PD-1 antibody, the RG1H10 anti-PD-1 antibody, or pembrolizumab. In other embodiments the ScFv moiety of the fusion protein encoded by an expression construct of a recombinant HSV can be derived from a monoclonal antibody that specifically binds PD-L1 such as the Combi5 anti-PD-L1 antibody, the H6B1LEM anti-PD-L1 antibody, or avelumab.

For example, in some embodiments an anti-PD-1 ScFv of an ScFv-Fc-TGFβtrap encoded by a recombinant HSV can include heavy chain CDRs having the amino acid sequences of SEQ ID NO:63 (HC-CDR1), SEQ ID NO:64 (HC-CDR2), and SEQ ID NO:65 (HC-CDR3), and light chain CDRs (LC-CDRs) having the amino acid sequences of SEQ ID NO:66 (LC-CDR1), SEQ ID NO:67 (LC-CDR2), and SEQ ID NO:68 (LC-CDR3), and can have a heavy chain variable domain with at least 95%. 96%, 97%, 98%, or 99% identity to SEQ ID NO:8, and a light chain variable domain with at least 95%, 96%. 97%, 98%, or 99% identity to SEQ ID NO:9. In some embodiments, the anti-PD-1 ScFv can comprise the heavy chain variable domain of the BB9 antibody (SEQ ID NO:8) and the light chain variable domain region of the BB9 antibody (SEQ ID NO:9). The linker of the ScFv can attach the N-terminus of the light chain to the C-terminus of the heavy chain or alternatively can attach the N-terminus of the heavy chain to the C-terminus of the light chain. In some embodiments the anti-PD-1 ScFv can comprise an amino acid sequence having at least 95%, 96%, 97%, 98%, or 99% identity to SEQ ID NO:11. In some embodiments the anti-PD-1 ScFv can be or comprise SEQ ID NO:11.

In further embodiments an anti-PD-1 ScFv of an ScFv-Fc-TGFβtrap encoded by a recombinant HSV can have a V_{H} region with at least 95%. 96%, 97%. 98%, or 99% identity to SEQ ID NO:12, and a V_{L} region with at least 95%, 96%, 97%, 98%, or 99% identity to SEQ ID NO:13. In some embodiments, the anti-PD-1 ScFv can comprise the V_{H} region of the RG1H10 antibody (SEQ ID NO:12) and the V_{L} region of the RG1H10 antibody (SEQ ID NO:13). The linker of the ScFv can attach the N-terminus of the light chain to the C-terminus of the heavy chain or alternatively can attach the N-terminus of the heavy chain to the C-terminus of the light chain. In some embodiments the anti-PD-1 ScFv can comprise an amino acid sequence having at least 95%, 96%. 97%, 98%, or 99% identity to SEQ ID NO: 15. In some embodiments the anti-PD-1 ScFv can be or comprise SEQ ID NO:15.

In some additional embodiments an anti-PD-1 ScFv of an ScFv-Fc-TGFβtrap encoded by a recombinant HSV can have a V_{H} region with at least 95%. 96%, 97%, 98%, or 99% identity to SEQ ID NO:16, and a V_{L} region with at least 95%. 96%, 97%. 98%, or 99% identity to SEQ ID NO:17. In some embodiments, the anti-PD-1 ScFv can comprise the V_{H} region of pembrolizumab (SEQ ID NO:16) and the V_{L}, region of pembrolizumab (SEQ ID NO:17). The linker of the ScFv can attach the N-terminus of the light chain to the C-terminus of the heavy chain or alternatively can attach the N-terminus of the heavy chain to the C-terminus of the light chain. In some embodiments the anti-PD-1 ScFv can comprise an amino acid sequence having at least 95%, 96%. 97%, 98%, or 99% identity to SEQ ID NO:19. In some embodiments the anti-PD-1 ScFv can be or comprise SEQ ID NO:19.

In some embodiments an anti-PD-L1 ScFv of an ScFv-Fc-TGFβtrap encoded by a recombinant HSV can have a V_{H} region with at least 95%, 96%, 97%, 98%, or 99% identity to SEQ ID NO:20, and a V_{L} region with at least 95%, 96%. 97%, 98%, or 99% identity to SEQ ID NO:21. In some embodiments, the anti-PD-L1 ScFv can comprise the V_{H} region of the Combi5 antibody (SEQ ID NO:20) and the V_{L} region of the Combi5 antibody (SEQ ID NO:21). The linker of the ScFv can attach the N-terminus of the light chain to the C-terminus of the heavy chain or alternatively can attach the N-terminus of the heavy chain to the C-terminus of the light chain. In some embodiments the anti-PD-L1 ScFv can comprise an amino acid sequence having at least 95%, 96%. 97%, 98%, or 99% identity to SEQ ID NO:23. In some embodiments the anti-PD-L1 ScFv can be or comprise SEQ ID NO:23.

In further embodiments an anti-PD-L1 ScFv can have a V_{H} region with at least 95%, 96%, 97%. 98%, or 99% identity to SEQ ID NO:24, and a VL region with at least 95%, 96%. 97%, 98%, or 99% identity to SEQ ID NO:25. In some embodiments, the anti-PD-L1 ScFv can comprise the V_{H} region of the H6B1LEM antibody (SEQ ID NO:24) and the V_{L} region of the H6B1LEM antibody (SEQ ID NO:25). In some embodiments the anti-PD-L1 ScFv can comprise an amino acid sequence having at least 95%, 96%, 97%, 98%, or 99% identity to SEQ ID NO:27. In some embodiments the anti-PD-L1 ScFv can be or comprise SEQ ID NO:27.

In some embodiments the anti-PD-L1 ScFv of an ScFv-Fc-TGFβtrap encoded by a recombinant HSV can have a V_{H} region with at least 95%, 96%, 97%, 98%, or 99% identity to SEQ ID NO:28, and a V_{L} region with at least 95%, 96%. 97%, 98%, or 99% identity to SEQ ID NO:29. In some embodiments, the anti-PD-L1 ScFv can comprise the V_{H} region of avelumab (SEQ ID NO:28) and the V_{L} region of avelumab (SEQ ID NO:29). In some embodiments the anti-PD-L1 ScFv can comprise an amino acid sequence having at least 95%, 96%, 97%, 98%, or 99% identity to SEQ ID NO:31. In some embodiments the anti-PD-1 ScFv can be or comprise SEQ ID NO:31.

TABLE 1
Monoclonal Antibodies that bind Immune
Checkpoint Proteins PD-1 and PD-L1

| | | LC variable | HC variable |
|---|---|---|---|
| | | region | region |
| Antibody | Antigen | SEQ ID NO | SEQ ID NO |
| BB9 | PD-1: human, mouse | 8 | 9 |
| RG1H10 | PD-1: human, mouse | 12 | 13 |
| Pembrolizumab | PD-1: human | 16 | 17 |
| Combi5 | PD-L1: human, mouse | 20 | 21 |
| H6B1LEM | PD-L1: human, mouse | 24 | 25 |
| Avelumab | PD-L1: human, mouse | 28 | 29 |

The TGFβ trap moiety of an ScFv-Fc-TGFβtrap fusion protein encoded by a recombinant oncolytic HSV as provided herein comprises the extracellular “ectodomain” of the transforming growth factor beta receptor II (TGFβRII)(Lin et al. (1995) J. Biol. Chem. 270:2747-2754), which may be referred to herein as the TGFβtrap or TGFβRII_{ecto}. The TGFβRII_{ecto} of an ScFv-Fc-TGFβtrap fusion protein encoded by a construct as disclosed herein is preferably the ectodomain of the human TGFβRII (SEQ ID NO:7) and can be a variant of the human TGFβRII ectodomain, for example, can have an amino acid sequence having at least 95%, 96%, 97%. 98%, or 99% identity to SEQ ID NO:7 that retains the TGFβ binding activity of the TGFβRII_{ecto} of SEQ ID NO:7.

The TGFβRII ectodomain is attached to the anti-PD-1 or anti-PD-L1 ScFv of an ScFv-Fc-TGFβtrap fusion protein via an Fc antibody region. In some embodiments the Fc region can be an Fc region of an IgG1 or IgG4 antibody (e.g., Genbank accession 6IFJ_A or Genbank accession CAA04843.1), and can be a human IgG1 or IgG4 Fc region or a variant thereof, for example, can be an Fc region comprising SEQ ID NO:2 or a sequence having at least 95% identity to SEQ ID NO:2, such as for example SEQ ID NO:3, or can be an Fc region comprising SEQ ID NO:5 or a sequence having at least 95% identity to SEQ ID NO:5. The Fc region can also be an Fc region of an IgG of a non-human species, or a variant thereof. For example, the Fc region used to connect the TGFβRII ectodomain to an scFv can be from a canine, equine, or feline species. In various embodiments the TGFβRII ectodomain is directly attached to a the antibody Fc region without an intervening peptide linker. In other embodiments the TGFβRII ectodomain is attached to the antibody Fc region by a sequence of between about four and about thirty-two amino acids, for example, between about four and about twenty-four amino acids, or between about four and about sixteen amino acids. For example, a (GGGGS)n (G4S) linker (SEQ ID NO: 62) can be used between the TGFβRII ectodomain and the Fc region of the ScFv-Fc-TGFβtrap fusion protein, where n can be between 1 and 30, between 1 and 24, between 1 and 12, between 1 and 8, between 1 and 6, or between 1 and 4, such as for example the linker (GGGGS)₃ of SEQ ID NO:56.

The ScFv-Fc-TGFβtrap fusion proteins disclosed herein are encoded by a single open reading frame and are designed for production and secretion by recombinant oncolytic HSV-infected cells, which may be cells in culture or cells of a subject treated with a recombinant HSV that includes a nucleic acid construct encoding an ScFv-Fc-TGFβtrap protein. Without limiting the compositions and methods provided herein to any particular mechanism, it is considered that an ScFv-Fc-TGFβtrap protein as disclosed herein can be produced by and secreted from infected cells and can dimerize via the Fc region of each polypeptide to form a two polypeptide molecule having two identical ScFvs that bind PD1 or PD-L1 and two TGFβRII ectodomains that bind TGFβ.

In various embodiments an ScFv-Fc-TGFβtrap fusion protein encoded by a nucleic acid construct will include a signal peptide for secretion of the fusion protein from the cell. The signal peptide can be any that directs secretion from a eukaryotic cell, such as a human cell, and can preferably be N-terminal to the ScFv of the fusion protein. One example of a signal peptide that may be used at the N-terminus of an ScFv-Fc-TGFβtrap fusion protein is SEQ ID NO:34. Additional nonlimiting examples of signal peptides that may be positioned at the N-terminus of an ScFv-Fc-TGFβtrap fusion protein encoded by a recombinant HSV include SEQ ID NO:35 and SEQ ID NO:36.

In various embodiments a construct encoding an ScFv-Fc-TGFβtrap fusion protein as provided herein can encode an ScFv-Fc-TGFβtrap fusion protein in which the ScFv is derived from anti-PD-1 antibody BB9 and can comprise the amino acid sequence of SEQ ID NO:40 or can be a variant of SEQ ID NO:40, such as a variant having at least 95%, 96%. 97%, 98%, or 99% identity to SEQ ID NO:40. In further embodiments a construct encoding an ScFv-Fc-TGFβtrap fusion protein as provided herein can encode an ScFv-Fc-TGFβtrap fusion protein in which the ScFv is derived from anti-PD-1 antibody RG1H10 and can comprise the amino acid sequence of SEQ ID NO:42 or can be a variant of the fusion protein of have the sequence of SEQ ID NO:42, such as a variant having at least 95%, 96%, 97%. 98%, or 99% identity to SEQ ID NO:42. In additional embodiments a construct encoding an ScFv-Fc-TGFβtrap fusion protein as provided herein can encode an ScFv-Fc-TGFβtrap fusion protein in which the ScFv is derived from pembrolizumab and can comprise the amino acid sequence of SEQ ID NO:44 or can be a variant of the protein of SEQ ID NO:44, such as a variant having at least 95%, 96%, 97%, 98%, or 99% identity to SEQ ID NO:44. Variants of anti-PD-1 ScFv-Fc-TGFβtrap fusion proteins include variants having alternative signal peptides, Fc regions, VH/VL orientations, and linkers connecting VH and VL regions or connecting the TGFβRII_{ecto} to the Fc region.

In yet further embodiments a construct encoding an ScFv-Fc-TGFβtrap fusion protein as provided herein can encode an ScFv-Fc-TGFβtrap fusion protein in which the ScFv is derived from anti-PD-L1 antibody Combi5 and can comprise the amino acid sequence of SEQ ID NO:46 or can be a variant of the polypeptide of SEQ ID NO:46, such as a variant having at least 95%, 96%, 97%. 98%, or 99% identity to SEQ ID NO:46. In other embodiments a construct encoding an ScFv-Fc-TGFβtrap fusion protein as provided herein can encode an ScFv-Fc-TGFβtrap fusion protein in which the ScFv is derived from anti-PD-1 antibody H6B1LEM and can comprise the amino acid sequence of SEQ ID NO:48 or can be a variant of the polypeptide of SEQ ID NO:48, such as a variant having at least 95%, 96%, 97%, 98%, or 99% identity to SEQ ID NO:48. In additional embodiments a construct encoding an ScFv-Fc-TGFβtrap fusion protein as provided herein can encode an ScFv-Fc-TGFβtrap fusion protein in which the ScFv is derived from avelumab and can comprise the amino acid sequence of SEQ ID NO:50 or can have the sequence of SEQ ID NO:50 or can be a variant of the polypeptide of SEQ ID NO:50, such as a variant having at least 95%, 96%, 97%. 98%, or 99% identity to SEQ ID NO:50. Variants of anti-PD-L1 ScFv-Fc-TGFβtrap fusion proteins include variants having alternative signal peptides, Fc regions, VH/VL orientations, and linkers connecting VH and VL regions or connecting the TGFβRII_{ecto} to the Fc region.

A construct encoding an ScFv-Fc-TGFβtrap fusion protein can be operably linked to a promoter for expression in a eukaryotic cell. Examples of promoters that can be used in a recombinant virus for expression of an ScFv-Fc-TGFβtrap fusion protein include, without limitation, a Cytomegalovirus (CMV) promoter (e.g., SEQ ID NO:33), a hybrid CMV promoter (e.g., U.S. Pat. No. 9,777,290), an HTLV promoter, an EF1a promoter, a hybrid EF1a/HTLV promoter (e.g., SEQ ID NO:32), a JeT promoter (U.S. Pat. No. 6,555,674), a SPARC promoter (e.g., U.S. Pat. No. 8,436,160), an RSV promoter, an SV40 promoter, or a retroviral LTR promoter such as an MMLV promoter, or a promoter derived from any of these. The construct can also include a polyadenylation sequence, such as, for example, a BGH, SV40, HGH, or RBG polyadenylation sequence. In some embodiments the polyadenylation sequence has the sequence of SEQ ID NO:38.

A recombinant oncolytic virus as provided herein can further include a gene encoding interleukin-12 (IL12). Thus, provided herein are engineered oncolytic viruses having a first gene encoding an ScFv-Fc-TGFβtrap fusion protein as disclosed above, in which the ScFv of the fusion protein binds an immune checkpoint inhibitor such as PD-1 or PD-L1, and at least a second gene encoding IL12. The IL12 gene can be human IL12 or can be an IL12 of another mammalian species. In its mature functional form IL12 is a heterodimer that includes the p40 subunits and the p35 subunit. A recombinant HSV as provided herein that includes a gene for expressing IL12 can include a sequence encoding the p40 polypeptide chain and a sequence encoding the p35 polypeptide chain, in which each sequence is independently operably linked to a separate promoter. Alternatively, a sequence encoding the p40 subunit can be linked to a sequence encoding the p35 subunit via a 2A “self-cleaving” sequence or an internal ribosome entry site (IRES) for the production of two polypeptide chains from the same transcriptional unit. Nonlimiting examples of 2A sequences include P2A, E2A, F2A and T2A (see for example Liu et al. (2017) Nature Sci Rep 7:2193). Nonlimiting examples of IRESs include those of MMLV, RSV, the FGF 1 and 2 genes, and the PDGF and VEGF genes (see for example Renaud-Gabardos et al. (2015) World J Exp Med 5:11-20; Mokrejs et al. (2006) Nuc Acids Res D125-D130). In further embodiments, the p40 subunit-encoding sequence can be linked to the p35-encoding sequence via a sequence encoding a peptide linker that allows for production of a single polypeptide encoding both subunits. For example, the IL12 gene can encode a single polypeptide (e.g., SEQ ID NO:54 or polypeptide having at least 95% identity thereto) comprising a murine IL12 p40 subunit attached to a murine IL12 p35 subunit via a 2× elastin linker.

The p40 IL12 subunit encoded by an IL12 gene as provided herein can comprise the human p40 IL12 subunit, i.e., can comprise SEQ ID NO:57, or can comprise an amino acid sequence having at least 95%, 96%, 97%, 98%, or 99% identity to SEQ ID NO:57. The p35 IL12 subunit encoded by an IL12 gene as provided herein can comprise the human p35 IL12 subunit, i.e., can comprise SEQ ID NO:58, or can comprise an amino acid sequence having at least 95%, 96%, 97%. 98%, or 99% identity to SEQ ID NO:58. In some embodiments, the IL12 gene of a recombinant HSV as provided herein includes a p40 IL12-encoding sequence followed by a peptide linker, followed by a p35-encoding sequence, where the IL12 gene encodes a polypeptide of SEQ ID NO:52 or a polypeptide having at least 95%, 96%, 97%, 98%, or 99% identity to SEQ ID NO:52.

An IL12 gene or IL12 subunit gene of a recombinant HSV as provided herein can be operably linked to a eukaryotic promoter, including, without limitation, any disclosed herein e.g., a CMV promoter (e.g., SEQ ID NO:33), a hybrid CMV promoter (e.g., U.S. Pat. No. 9,777,290), an HTLV promoter, an EF1α promoter, a hybrid EF1α/HTLV promoter (e.g., SEQ ID NO:32), a JeT promoter (U.S. Pat. No. 6,555,674), a SPARC promoter (e.g., U.S. Pat. No. 8,436,160), an RSV promoter, an SV40 promoter, or a retroviral LTR promoter, or a promoter derived from any of these. The promoter operably linked to the IL12 gene(s) can be the same as or different from the promoter operably linked to the gene encoding the ScFv-Fc-TGFβtrap fusion protein. The IL12-encoding construct can also include a polyadenylation sequence, such as, for example, a BGH, SV40, HGH, or RBG polyadenylation sequence. In some embodiments the polyadenylation sequence has the sequence of SEQ ID NO:38.

A dual gene recombinant HSV that includes a gene encoding an ScFv-Fc-TGFβtrap fusion protein and a gene encoding IL12 can include a gene encoding any ScFv-Fc-TGFβtrap disclosed herein. For example, a dual gene recombinant HSV can include an IL12 gene and a gene encoding an ScFv-Fc-TGFβtrap protein in which the ScFv of the ScFv-Fc-TGFβtrap protein specifically binds PD-1. In various embodiments the ScFv of the ScFv-Fc-TGFβtrap protein of the dual gene vector can be derived from a BB9 antibody, an RG1H10 antibody, or pembrolizumab, for example. In some embodiments, the ScFv-Fc-TGFβtrap protein encoded by a dual gene vector comprises SEQ ID NO:40, SEQ ID NO:42, or SEQ ID NO:44, or comprises an amino acid sequence having at least 95%, 96%. 97%, 98%, or 99% identity to SEQ ID NO:40, SEQ ID NO:42, or SEQ ID NO:44. In other embodiments, a dual gene recombinant HSV can include an IL12 gene and a gene encoding an ScFv-Fc-TGFβtrap protein in which the ScFv of the ScFv-Fc-TGFβtrap protein specifically binds PD-L1. For example, the ScFv of the ScFv-Fc-TGFβtrap protein of the dual gene vector can be derived from a Combi antibody, an H6B1LEM antibody, or avelumab. In some embodiments, the ScFv-Fc-TGFβtrap protein encoded by a dual gene vector comprises SEQ ID NO:46, SEQ ID NO:48, or SEQ ID NO:50, or comprises an amino acid sequence having at least 95%, 96%, 97%, 98%, or 99% identity to SEQ ID NO:46, SEQ ID NO:48, or SEQ ID NO:50.

In any of the embodiments contemplated herein, the ScFv-Fc-TGFβtrap encoding gene and the IL12-encoding gene can be oriented for transcription in the same direction and transcribed off of the same strand of the viral genome, or the two genes can be oriented for transcription in opposite directions, such that the genes are transcribed off of opposite strands of the viral genome. In any of the embodiments contemplated herein, the ScFv-Fc-TGFβtrap encoding gene and the IL12-encoding gene can be inserted at the same locus of the HSV genome, for example, can be positioned adjacent to one another in the genome, or may the two transgenes be inserted at different genome loci. In some examples, one or both of the ScFv-Fc-TGFβtrap and IL12 genes is inserted into a gene locus, where the gene is functionally deleted. For example, one or both of the ScFv-Fc-TGFβtrap and IL12 genes can be inserted into the gene encoding ICP6, the gene encoding ICPO, the gene encoding vhs, the gene encoding ICP27, or the RL1 gene that encodes ICP34.5. In some embodiments, both of the ScFv-Fc-TGFβtrap and IL12 genes are inserted into both copies of the RL1 gene.

In various embodiments, a dual gene recombinant HSV that includes a gene encoding an ScFv-Fc-TGFβtrap fusion protein and a gene encoding IL12 can include a gene encoding a ScFv-Fc-TGFβtrap having at least 95% identity to SEQ ID NO:40, SEQ ID NO:42, or SEQ ID NO:44, in which the ScFv-Fc-TGFβtrap protein disrupts PD1/PD-L1 signaling and binds TGFβ, and a gene encoding IL12 having at least 95% identity to SEQ ID NO:54. For example, a recombinant HSV as provided herein can include a gene encoding an ScFv-Fc-TGFβtrap of SEQ ID NO:40, SEQ ID NO:42, or SEQ ID NO:44 and can encode an IL12 of SEQ ID NO:54. In further embodiments, a dual gene recombinant HSV that includes a gene encoding an ScFv-Fc-TGFβtrap fusion protein and a gene encoding IL12 can include a gene encoding a ScFv-Fc-TGFβtrap having at least 95% identity to SEQ ID NO:46, SEQ ID NO:48, or SEQ ID NO:50, where the ScFv-Fc-TGFβtrap protein disrupts PD1/PD-L1 signaling and binds TGFβ, and a gene encoding IL12 having at least 95% identity to SEQ ID NO:54. For example, a recombinant HSV as provided herein can include a gene encoding an ScFv-Fc-TGFβtrap of SEQ ID NO:46, SEQ ID NO:48, or SEQ ID NO:50 and can encode an IL12 of SEQ ID NO:54.

The dual gene recombinant HSV according to any of the embodiments provided herein may be an HSV-1 strain or an HSV-2 strain, and in some preferred embodiments is derived from HSV-1 strain F, HSV-1 strain KOS, HSV-1 strain JS1, or HSV-1 strain 17. In various embodiments the HSV-1 strain does not include a functional ICP34.5-encoding gene. In some embodiments the recombinant HSV is a derivative of HSV1716 (Seprehvir®) (WO 92/13943; MacClean et al. (1991) J. Gen. Virol. 72:631-639; Brown et al. (1992) J. Gen. Virol. 75:2367-2377).

In further examples, the HSV into which the ScFv-Fc-TGFβtrap gene and the IL12 gene is introduced can be Seprehvec®, an HSV-1 strain 17-derived HSV vector that lacks a functional ICP34.5-encoding gene, where the ICP34.5-encoding genes have a 695 bp deletion. The TRL region of the HSV genome that includes the first RL1 gene (nucleotide positions 513-1259) includes a deletion from position 382 through 1076 and the IRL region of the HSV genome that includes the second RL1 gene (nucleotide positions 125858-12112) includes a deletion from position 125992 through 125298. The sites of the deletions in the ICP34.5-encoding RL1 genes are also the insertion sites for the constructs provided herein.

Thus, in various exemplary embodiments the recombinant HSVs provided herein include a transgene encoding an ScFv-Fc-TGFβtrap and a transgene encoding IL12 inserted at the ICP34.5-encoding gene locus, where both ICP34.5-encoding genes of the HSV are inactivated and both ICP34.5-encoding genes have insertions of the ScFv-Fc-TGFβtrap and IL12-encoding transgenes. The recombinant HSV can have additional alterations of the viral genome, for example can have one or more additional endogenous viral genes that are mutated, including functionally deleted. The ScFv-Fc-TGFβtrap gene and IL12 gene can be regulated by separate promoters. The genes can be oriented in the same or opposite directions, e.g., in some embodiments, the genes are transcribed from opposite strands of the viral genome. In some embodiments the recombinant HSV is a Seprehvec® HSV-1 derivative, having a 695 bp internal deletion in both copies of the ICP34.5-encoding gene, and the ScFv-Fc-TGFβtrap and IL12-encoding transgenes are each inserted into both non-functional ICP34.5-encoding gene loci oriented for transcription in opposite directions.

Recombinant HSVs provided herein can be made using molecular cloning techniques known in the art, including restriction endonuclease digestion, ligation, polymerase chain reaction (PCR), and/or gene synthesis (e.g., using commercial service such as DNA 2.0, Blue Heron, Genewiz, GeneScript, Synbio Technologies, GeneArt, etc.). Insertion of nucleic acid constructs into an HSV genome, which can be for example from about 135 to about 150 kb in size can utilize homologous recombination, including using site-specific recombinases (see, for example, U.S. Pat. No. 9,085,777, incorporated herein by reference) and/or can use cas/CRISPR methods. The genome sequences of multiple HSV strains are publicly available, including the sequence of HSV-1 strain 17 (human alphaherpesvirus 1 strain 17; (GenBank: LT576870.1).

For the production of virus, a viral genome (for example a recombinant viral genome into which one or more modifications (insertions, deletions, or sequence alterations) has been made, e.g., into which one or more nucleic acid constructs has been inserted, can be transfected into cells that are able to produce virus, such as, for example, Vero cells, BHK cell, A431 cells, or HepG2 cells. Recombinant virus can be isolated from plaques of plated infected cells.

Recombinant viruses as provided herein can be used for the treatment of cancer. Viruses may be formulated as pharmaceutical compositions where the viruses may be combined with a pharmaceutically acceptable carrier, diluent, or adjuvant. The carrier can include an alcohol such as ethanol, a sugar or sugar alcohol (e.g., inositol, sorbitol, mannitol), a polyol (e.g., glycerol, propylene glycol, polyethylene glycol), suitable mixtures thereof, a protein or peptide (serum albumin, gelatin, etc.) and/or a lipid or surfactant. An aqueous solution for parenteral, intravenous, intra-arterial, intramuscular, subcutaneous, intratumoral, peritumoral, and intraperitoneal administration, for example, or for catheter delivery can include buffering agents and can include salts and/or sugars such that the composition is isotonic. Pharmaceutically-acceptable salts include inorganic acids such as, for example, hydrochloric or phosphoric acids, or such organic acids as acetic, oxalic, tartaric, mandelic, and the like. Salts include, for example, sodium, potassium, ammonium, calcium, or ferric hydroxides or chlorides, and salts of organic bases as isopropylamine, trimethylamine, histidine, procaine and the like. The pharmaceutical formulation for administration to a human subject will be sterile and can optionally include a preservative, antibacterial agent, and/or antifungal agent.

The pharmaceutical virus preparation can include the recombinant HSV at a titer of about 10⁶ pfu per ml, greater that 10⁶ pfu per ml, 10⁷ pfu per ml, greater than 10⁷ pfu per ml, 10⁸ pfu per ml, or greater than 10⁸ pfu per ml, for example. The pharmaceutical formulation can be provided in vials and may be provided in frozen form and can optionally include a cryoprotectant such as, for example, glycerol or gelatin.

### Methods of Treating Cancer

Also provided is a method of treating cancer using a recombinant HSV as provided herein. The method can include administering an effective amount of a recombinant HSV to a subject having cancer. The recombinant HSV can be administered as a pharmaceutical composition as provided herein. For example, the pharmaceutical composition can be an injectable solution that in some embodiments is injected into the tumor or in the vicinity of the tumor. Alternatively or in addition, the recombinant HSV can be administered intravenously or intra-arterially (see for example, US 2020/0078426, incorporated herein by reference).

In some embodiments, provided herein are methods of treating cancer in a subject using a recombinant HSV that encodes an ScFv-Fc-TGFβtrap fusion protein as provided herein, in which the progression of a tumor is reduced in subjects receiving the recombinant HSV encoding the ScFv-Fc-TGFβtrap fusion protein with respect subjects receiving a control virus that does not encode the ScFv-Fc-TGFβtrap fusion protein. Treatment with the recombinant HSV may result in reduced tumor growth or spread or a reduced rate of tumor growth or spread. In some embodiments, treating cancer in a subject using a recombinant HSV that encodes an ScFv-Fc-TGFβtrap fusion protein as provided herein can increase survivorship with respect to treatment with a control virus that does not encode the ScFv-Fc-TGFβtrap fusion protein. In some embodiments, treating cancer in a subject using a recombinant HSV that encodes an ScFv-Fc-TGFβtrap fusion protein as provided herein can decrease tumor recurrence with respect to treatment with a control virus that does not encode the ScFv-Fc-TGFβtrap fusion protein.

Viruses according to aspects of the present invention may be administered by any of a number of routes, including but not limited to, parenteral, intravenous, intra-arterial, intramuscular, intratumoral, peritumoral, and oral. HSVs may in some embodiments be formulated in a liquid composition for administration by injection to a selected region of the subject's body. For example, HSVs may be administered to a body cavity (intracavitary administration, e.g. intrapleural, intrapulmonary, or intraperitoneal) which can optionally use a drain or catheter inserted into the patient. Administration may also be by intratumoral or peritumoral delivery, which may be by injection. Administration of oncolytic herpes simplex virus may be locoregional administration, e.g. to a localized region of the body in which the tumor is present.

Alternatively, administration of oncolytic herpes simplex virus may be by infusion to the blood, e.g. intravenous or intra-arterial infusion, and the virus may be formulated for such administration. Infusion of the formulated viral composition to the blood may take between about 30 minutes and about 3 hours, for example about 1 hour, about 2 hours or about 3 hours. Intravenous administration may comprise infusion into the venous system in close proximity to the location or locations of the cancer, e.g. head and neck cancer.

Infusion to the blood is preferably at a peripheral site, e.g. to a vein or artery near the surface of the skin and not within deep tissue. Examples of suitable peripheral locations are veins in the arm or leg. In some related embodiments, administration may be via a central venous line. Administration is preferably non-invasive, e.g. does not require a surgical, invasive or interventional radiological procedure in order to locate a specific vein or artery within deep tissue or proximal to internal organs. In some embodiments, the subject may have a peripheral venous device, catheter or cannula fitted in order to facilitate the administration. Preferably, administration can be performed in an out-patient setting.

The administered HSV can be any recombinant HSV disclosed herein, such as, for example, any that includes a nucleic acid construct that encodes an ScFv-Fc-TGFβtrap that is able to bind an immune checkpoint inhibitor such as PD-1 or PD-L1 and is able to bind TGFβ. The recombinant HSV used for treating cancer can include an IL12 gene in addition to an ScFv-Fc-TGFβtrap that specifically binds PD-1 or PD-L1.

A subject to be treated may be any animal or human. In various embodiments the subject is human and may be a child. A subject may have been diagnosed with a cancer or be suspected of having a cancer. A subject may have been previously treated for cancer. In further embodiments the subject may be a non-human animal such as, but not limited to, a dog, a cat, or a horse.

A cancer may be a neoplasm or tumor. A neoplasm or tumor may be any abnormal growth or proliferation of cells and may be located in any tissue. The cancer may be benign or malignant and may be primary or secondary (metastatic). The cancer can be without limitation, bladder, bone, breast, eye, stomach, head and neck, kidney, liver, lung, ovarian, pancreatic, prostate, skin, or uterine cancer, a mesothelioma, a glioma, a neurocytoma, or a chondrosarcoma. Cancers to be treated may include non-CNS solid tumor, sarcoma, chordoma, clival chordoma, peripheral nerve sheath tumor, malignant peripheral nerve sheath tumor or renal cell carcinoma. In some embodiments the cancer may be a solid tumor. Solid tumors may, for example, be in bladder, bone, breast, eye, stomach, head and neck, germ cell, kidney, liver, lung, nervous tissue, ovary, pancreas, prostate, skin, soft-tissues, adrenal gland, nasopharynx, thyroid, retina, and uterus. Solid tumors may include melanoma, rhabdomyosarcoma, Ewing sarcoma, and neuroblastoma. The cancer may be a pediatric solid tumor, i.e. solid tumor in a child, for example osteosarcoma, chondroblastoma, chondrosarcoma, Ewing sarcoma, malignant germ cell tumor, Wilms tumor, malignant rhabdoid tumor, hepatoblastoma, hepatocellular carcinoma, neuroblastoma, melanoma, adrenocorticoid carcinoma, nasopharyngeal carcinoma, thyroid carcinoma, retinoblastoma, soft-tissue sarcoma, rhabdomyosarcoma, desmoid tumor, fibrosarcoma, liposarcoma, malignant fibrous histiocytoma, or neurofibrosarcoma.

The administering can be by any means and can be, as nonlimiting examples, parenteral, systemic, intracavitary, intrapulmonary, intraperitoneal, peritumoral, or intratumoral, and may be by injection, intravenous or intra-arterial infusion, catheter, or other delivery means. Injection can be, for example, parenteral, subcutaneous, intramuscular, intravenous, intra-arterial, intratumoral, or peritumoral. In some embodiments treatment with the recombinant HSV may be by administration of the virus to a body cavity (intracavitary administration, e.g. intrapulmonary or intraperitoneal) and may involve administration via a catheter or drain inserted in the patient. Administration of virus may follow complete or partial drainage of effusion fluid from the body cavity. The virus may be administered as a fluid formulation. The treatment regimen may include more than one administration of the virus and can include multiple dosings over a period of hours, days, weeks, or months. The treatment regimen can precede or follow any other cancer treatment.

### Recombinant Fusion Protein Compositions

A further aspect of the invention is a composition that includes an ScFv-Fc-TGFβtrap polypeptide. The composition can be, for example, virus-free conditioned media (VFCM) prepared from a culture of cells infected with a recombinant HSV as provided herein that includes a nucleic acid construct for the expression of the ScFv-Fc-TGFβtrap in host cells. The composition can include, as nonlimiting examples, an ScFv-Fc-TGFβtrap protein comprising SEQ ID NO:40, SEQ ID NO:42, SEQ ID NO:44, SEQ ID NO:46, SEQ ID NO:48 or SEQ ID NO:50, or a variant of any of these having at least 95% identity to SEQ ID NO:40, SEQ ID NO:42, SEQ ID NO:44, SEQ ID NO:46, SEQ ID NO:48 or SEQ ID NO:50, where the ScFv-Fc-TGFβtrap protein disrupts PD1/PD-L1 signaling and binds TGFβ. In some embodiments the composition can comprise a ScFv-Fc-TGFβtrap having an scFv based on the BB9 PD-1 antibody, e.g., comprising SEQ ID NO:40 or an amino acid sequence having at least 95%, 96%, 97%, 98% or 99% identity thereto. In some embodiments the composition can comprise a ScFv-Fc-TGFβtrap having an scFv based on the Combi5 PD-L1 antibody, e.g., comprising SEQ ID NO:46 or an amino acid sequence having at least 95%, 96%, 97%, 98% or 99% identity thereto. For example, the VFCM can be produced from an HSV such as SepGI-097 or SepGI-138 as disclosed herein, or an HSV substantially similar thereto. The virus-free conditioned media can optionally further include IL12. For example, the VFCM can be produced from an HSV such as SepGI-143 or SepGI-162 as disclosed herein, or an HSV substantially similar thereto, or can be produced from an HSV such as SepGI-145 or SepGI-167 as disclosed herein, or an HSV substantially similar thereto. A VFCM composition as provided herein can be prepared by methods disclosed in the examples, by harvesting media from infected cultures, centrifuging to remove cells and cell debris, and filtration to remove virus.

In some embodiments, a VFCM can be used in methods of cancer treatment. For example, in some embodiments a VFCM can be used in veterinary applications, where a nonhuman animal such as but not limited to a dog, a horse, a cow, a bull, a monkey, an ape, or a feline having cancer is administered a VFCM composition that includes.

The cancer can be any type of cancer, such as but not limited to a soft tissue sarcoma, osteosarcoma, melanoma, or renal carcinoma). In various embodiments, methods are provided for slowing or halting the progression of cancer, reducing tumor size, preventing recurrence of cancer, or extending survivorship of a subject, such as a non-human subject, by administering a VFCM as provided herein.

The VFCM composition for administration to a subject can be a VFCM that is concentrated, dialyzed, or diluted. One or more components of the VFCM may be removed from the VFCM, for example by capture or chromatography and one or more compounds may be added to the VFCM, including but not limited to one or more pharmaceutical excipients (such as but not limited to, salts, buffering agents, stabilizers) or one or more therapeutic compounds. In some embodiments, a VFCM composition can be used to prepare isolated, partially purified, or substantially purified ScFv-Fc-TGFβtrap protein. Partial or substantial purification can be by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution, or any combinations of these. For example, the conditioned media can be subjected to chromatography and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In some embodiments, affinity chromatography comprises protein A or G (cell wall components from Staphylococcus aureus).

After purification, polypeptides may be exchanged into different buffers and/or concentrated by any of a variety of methods known to the art, including, but not limited to, filtration and dialysis.

A purified protein composition (including a substantially purified protein composition) that comprises an ScFv-Fc-TGFβtrap and, optionally, IL12, can be formulated for pharmaceutical use and can be used for the treatment of cancer. Administration may be local, as disclosed hereinabove for a viral formulation, or may be systemic, and is preferably in a therapeutically effective amount. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

### EXAMPLES

### Example 1. ScFv-Fc-TGFβtrap Constructs

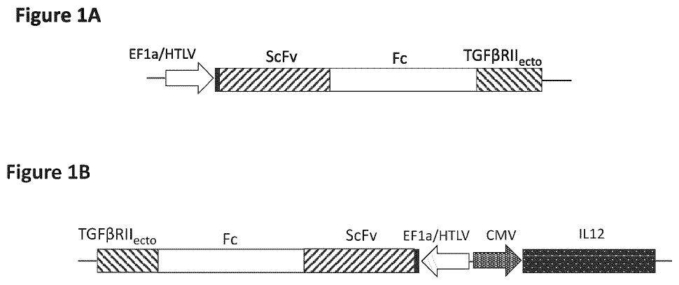

ScFv-Fc-TGFβtrap constructs were designed for expressing a fusion protein that included, proceeding from the N-terminal end to the C-terminal end of the fusion proteins: a signal peptide (SEQ ID NO:34), an ScFv antibody that specifically binds either PD-L1 or PD-1, the human IgG4 Fc region (Fc4, SEQ ID NO:2), and the extracellular domain of the human TGFβ receptor II (TGFβRII ectodomain or TGFβRII_{ecto}, SEQ ID NO:7). FIG. 1A provides a general diagram of the constructs which included the EF1a/HTLV hybrid promoter (SEQ ID NO:32) operably linked to the fusion protein-encoding sequence. Three ScFv-Fc-TGFβtrap constructs were assembled that included sequences encoding ScFv anti-PD-1 antibodies: a construct that included a sequence encoding an ScFv of anti-PD-1 antibody BB9 (SEQ ID NO:39), a construct that a sequence encoding an ScFv of anti-PD-1 antibody RG1H10 (SEQ ID NO:41), and a construct that included a sequence encoding an ScFv having an amino acid sequence derived from the anti-PD-1 antibody pembrolizumab (SEQ ID NO:43). The anti-PD-1 monoclonal antibodies BB9 and RG1H10 are reactive against mouse as well as human PD-1 (Table 1). In addition, three constructs were built that included ScFv antibodies to PD-L1: a construct that included a sequence encoding an ScFv of anti-PD-L1 antibody H6B1LEM (SEQ ID NO:45), a construct that included a sequence encoding an ScFv of anti-PD-L1 antibody Combi5 (SEQ ID NO:47), and a construct that included a sequence encoding an ScFv having an amino acid sequence derived from avelumab (SEQ ID NO:49). The anti-PD-L1 monoclonal antibody H6B1LEM, the anti-PD-L1 monoclonal antibody Combi5 and the anti-PD-L1 monoclonal antibody avelumab are reactive against mouse as well as human PD-L1 (Table 1).

TABLE 2
ScFv components of antibody-Fc-TGFβRII Fusion Proteins

| | | | ScFv-Fc-TGFβtrap |
|---|---|---|---|
| HSV | | ScFv | Fusion protein |
| Strain | ScFv antibody source | SEQ ID NO | SEQ ID NO |
| SepGI-097 | BB9 anti-PD-1 | 11 | 40 |
| SepGI-152 | RG1H10 anti-PD-1 | 15 | 42 |
| SepGI-138 | Combi5 anti-PD-L1 | 23 | 46 |
| SepGI-137 | H6B1LEM anti-PD-L1 | 27 | 48 |

Constructs flanked by attL sites were generated by PCR cloning and inserted into the internally deleted RL1 locus of the HSV-1 Seprehvec® genome. Seprehvec® is an HSV-1 vector derived from HSV strain 17 in which both copies of the RL1 gene that encodes the γ34.5 kd (ICP34.5) polypeptide responsible for neurovirulence are disrupted by a 695 bp deletion (nucleotides 125975 to 125221 within the RL1 sequence) that inactivates the RL1 gene. The RL1 deletion site includes attR recombination sites for insertion of any gene or construct of interest flanked by attL sequences. The ScFv-Fc4-TGFβRII_{ecto} constructs flanked by attL sequences were inserted into both RL1 loci at the deletion sites using in vitro recombinational cloning that used the LR Clonase™ Plus enzyme mixture of Integrase and Integration Host Factor (ThermoFisher, Carlsbad, CA) essentially according to the manufacturer's instructions.

Following the recombination reaction, viral genomic DNA was transfected into BHK (Baby Hamster Kidney fibroblast) cells for the production of recombinant virus. Virus was harvested from transfected BHK cells, then used to infect Vero (African Green Monkey (Chlorocebus sp.) kidney epithelial) cells. Individual plaques from infected Vero cells were collected and passaged to new Vero cells. This process was repeated for a total of four rounds of plaque isolation. Virus stocks were then generated by infection of ˜3.2×10⁷ BHK cells with ˜3.2×10⁵ plaque forming units (PFU) of virus and culturing for three days. After three days, supernatants were spun twice at 2100×g to pellet cells and debris. After pelleting the cells, the supernatant containing virus was spun at 17200×g to pellet virus. Virus was resuspended, filtered, and titered on Vero cells. Viral seed stocks and research stocks were produced from purified ScFv-Fc-TGFβtrap viruses SepGI-097 (anti-PD-1-Fc4-TGFβtrap), SepGI-137 (anti-PD-L1-Fc4-TGFβtrap), SepGI-138 (anti-PD-L1-Fc4-TGFβtrap), and SepGI-152 (anti-PD-1-Fc4-TGFβtrap).

### Example 2. Quantitation of TGFβRII_{ecto} Produced by Recombinant ScFv-Fc-TGFβTrap Viruses

To test production of the ScFv-Fc-TGFβtrap by virally-infected cells, the SepGI-097 (anti-PD-1-Fc4-TGFβtrap), SepGI-137 (anti-PD-L1-Fc4-TGFβtrap), and SepGI-138 (anti-PD-L1-Fc4-TGFβtrap) viruses were used to infect A431 human epidermoid carcinoma cells and HepG2 human liver cancer cells.

The engineered ScFv-Fc-TGFβtrap constructs described in Example 1 included an N-terminal signal peptide (SEQ ID NO:34) to direct secretion of the fusion proteins from infected host cells (FIG. 1A). To determine the amount of the TGFβRII ectodomain (TGFβRII_{ecto}) produced by the transgenic viruses, the conditioned medium of host cells infected with the engineered strains was analyzed. Meso Scale Discovery (MSD) sandwich assays (Meso Scale Discovery, Rockville, MD; Dabitao et al. (2011) J Immunol Methods 372:71-77) were performed using capture and detection antibodies that recognize TGFβRII (Human TGFβ-RII Duo Set ELISA kit, R&D Systems, Minneapolis, MN). The goat anti-human TGFβRII capture antibody (R&D Systems part #842376) was used to coat wells of a 96 well plate, and a biotinylated goat anti-human TGFβRII antibody (R&D Systems part #842377) was used in combination with MSD Sulfo-Tag labeled streptavidin (Meso Scale Diagnostics, Rockville, MD) for detection.

To generate virus-free conditioned medium (VFCM) from ScFv-Fc-TGFβtrap HSV strains, 12-well plates were seeded with 3×10⁵ A431 cells, or, in separate plates, HepG2 cells, in 1 mL of medium at 370 C, 5% CO₂. The next day, the A431 cells and HepG2 cells were infected with recombinant HSVs at MOI=0.5 and incubated for 3 days in 1.25 mL of medium. After 3 days, cell supernatants were removed and filtered through 0.1 μm membranes (Pall Acrodisc Syringe filter part #4611) to remove virus. The post-viral culture supernatants (VFCMs) were then aliquoted and stored at −80° C. VFCMs of SepGI-Null, the Seprehvec® HSV vector not including an exogenous transgene was also prepared as a control.

The assays for quantitation of the TGFβRII ectodomain were performed by diluting the capture antibody in Dulbecco's PBS (DPBS) to make a 4 μg per mL stock solution and adding 30 μl of the stock solution to the bottom corner of each well of a 96 well plate. The plate was tapped gently to ensure the antibody solution covered the bottom of each well evenly, and then the plate was incubated on a see-saw shaker for 5-10 minutes, after which the plate was sealed and incubated overnight at 4° C. without shaking. The wells were then blocked with 150 μl 5% MSD Blocker A (Meso Scale Discovery, Rockville, MD) in 0.05% Tween 20, 1×DPBS for 1.5 h at room temperature or overnight at 4° C. on an orbital shaker. The plates were then washed three times with 1×DPBS, 0.05% Tween20, after which dilutions (e.g., 1:10, 1:50, 1:250 dilutions) of the VFCM samples were added to well in a volume of 50 μl. In addition to VFCM of SepGI-Null (lacking a transgene), conditioned medium from uninfected cells was used as a further control. Serial dilutions of an 80 ng/ml TGFβRII standard (provided in the R&D ELISA kit) were added to separate wells. The plate was sealed and incubated on an orbital shaker for 2 hours at room temperature. Following this incubation, the plate was washed three times with PBS/0.05% Tween20 (PBS-T) after which 25 μl of the detection solution (1 μg/ml biotinylated TGFβRII detection antibody and 1 μg/ml Sulfo-Tag-labeled streptavidin reagent in PBS-T/1% Blocker A) was added. The plate was sealed, covered with foil, and incubated on an orbital shaker for 1 h at room temperature. The plate was then washed three times with PBS-T, 150 μl of Read Buffer was added to each well, and the plate was read on an MSD imager according to the manufacturer's instructions (Meso Scale Discovery).

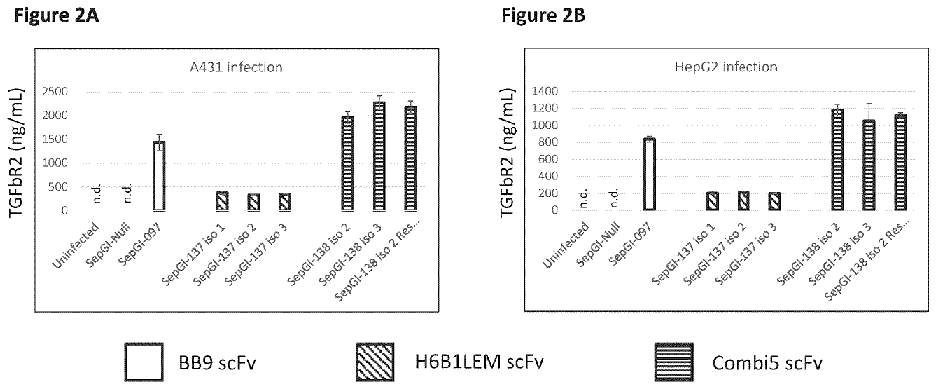

FIGS. 2A and 2B provide the results of the assays as the average of two wells per sample in ng/mL based on interpolation from the standard curve dilution series of recombinant human TGFβRII. FIG. 2A shows the amount of TGFβRII antibody-binding molecule determined from the MSD assay of VFCMs of infected A431 cells and FIG. 2B provides the amount of TGFβRII antibody-binding molecule determined from the MSD assay of VFCMs of infected HepG2 cells Results from assaying the VFCM from two cell types were remarkably consistent, demonstrating that all of the cell samples infected with viruses that included ScFv-Fc-TGFβtrap (SepGI-097, SepGI-137, and SepGI-138) produced molecules reactive with the TGFβRII antibodies (i.e., produced ScFv-Fc-TGFβtrap molecules), where SepGI-138 infection resulted in the greatest amount of TGFβRII-containing molecules being produced.

### Example 3. CD103 Cell-Based Assay for TGFβRII Trap Function

TGFβ1 induces CD103 (αE integrin) expression by cytotoxic T cells (Wang et al. (2004) J Immunol 172:214-221; El-Asady et al. (2005) J Exp Med 201:1647-1657). To determine the degree to which expression of ScFv-FcTGFβtrap constructs blocked TGFβ signaling, an assay was developed to assess the ability of ScFv-Fc-TGFβtrap fusion proteins to deplete the cell medium of TGFβ by measuring lack of expression of CD103 from the surface of CD8+ T cells.

Isolation of T cells from PBMCs was performed using the EasySep™ Human T Cell Isolation kit from STEMCELL Technologies (Vancouver, BC, Canada; catalog number 17951). Briefly, thawed PBMCs (approximately 5×10⁷ cells) were added to 9 mL pre-warmed culture medium and pelleted at 1,400 rpm for 5 minutes, and then resuspended in 1 ml Robosep buffer (STEMCELL Technologies, catalog number 20104) and transferred to a 5 ml polystyrene tube. Fifty μl of Antibody Cocktail from the EasySep™ Human T Cell Isolation kit was then added to the cells, and the cells plus antibody mixture were mixed by pipetting and incubated for 10 min at room temperature. Beads provided with the EasySep™ kit were vortexed and 40 μl of the bead suspension were added to the cell-antibody mixture. After 2-3 minutes, EasySep™ buffer was added to the tube to reach a total volume of 2.5 mls. The entire 2.5 mls that included cells, antibodies, and beads was added to the Robosep magnet and allowed to stand for 3 min, after which buffer and unbound cells were removed with a 2 ml serological pipet and added to a 15 ml conical tube. Complete medium was added to bring the volume to 10 ml and the tube was centrifuged at 1,400 rpm for 4 minutes. After removing the supernatant, the cell pellet was resuspended in 4 ml AIM-V medium (ThermoFisher, catalog number 12055091) and the cells were counted. Cell were either frozen for future use or used directly following CD3/CD28 selection as described below.

VFCMs of A431 cells and HepG2 cells infected with the ScFv-Fc4-TGFβtrap viruses SepGI-097, SepGI-137, and SepGI-138 were tested for TGFβtrap function in the cell-based CD103 expression assay. VFCM of SepGI-Null, the Seprehvec® vector without any exogenous transgene, and conditioned medium from uninfected cells were used as controls.

Fifty μl of a 4 ng/mL solution of recombinant human TGFβ1 (rhTGFβ; Sino Biological, Wayne, PA) was added to each well followed by the addition of 100 μl of VFCM undiluted or diluted 1:5 in RPMI media with 1% FBS. Each assay was performed in duplicate. Control wells received 100 μl of AIM-V medium that included recombinant human TGFβRII (Sino Biologicals, catalog #103358-H03H) titrated from 6000 ng/mL to 8 ng/mL plus 2.5 μg/mL of an IgG directed toward either PD-1 or PD-L1.

After adding rhTGFβ and ScFv-Fc4-TGFβtrap HSV VFCMs to the wells, the plate was pre-incubated at 37° C., 5% CO₂ while the T cells were further prepared. T cells were spun down and resuspended at a concentration of 1.6×10⁶ cells per mL in AIM-V medium. Anti-CD3/CD28 magnetic beads (Human T cell Activator Dynabeads, ThermoFisher, Carlsbad, CA) sufficient to provide a 3:1 ratio of T cells to beads in the assays (8×10¹ T cells per assay) were washed by suspending the beads in 1 ml AIM-V medium and using the Robosep magnet for capture. The beads were then suspended in a volume of 100-200 μl AIM-V medium and transferred to the resuspended T cells. The tube was mixed by inversion and 50 μl of T cells plus beads were added to the wells of the 96 well assay plate that included rhTGFβ and VFCM. The plate was then incubated for two days (if fresh T cells were used) or three days (if frozen T cells were used) at 37° C., 5% CO₂.

For staining of cells for flow cytometry analysis, the plate was centrifuged at 1,500 rpm for 5 min, the supernatants were removed, the plate was briefly vortexed, 80 μl of staining mix was added to each well, and the mixture was pipetted up and down in the wells. Staining mix included 140 μl CD4-PECY7 (BioLegend #357410, Clone #A161A1), 140 μl CD8-AF488 (BioLegend #300916, Clone #HIT8a), and 280 μl CD103-PE (BioLegend#350206, Clone #Ber-ACT8) in 10.5 ml FACS buffer (DPBS+2% FBS, 1 mM EDTA). The plates were then incubated in the dark on ice or at 4° C. for 20 min. Following incubation, 100 μl of FACS buffer was added to each well and the plate was centrifuged at 1,500 rpm for 4 min. The wash was repeated using 200 μl FACS buffer and the cells were finally resuspended in 180 μl and analyzed by flow cytometry using an Attune NxT flow cytometer (Thermo Fisher) essentially according to the manufacturer's instructions.

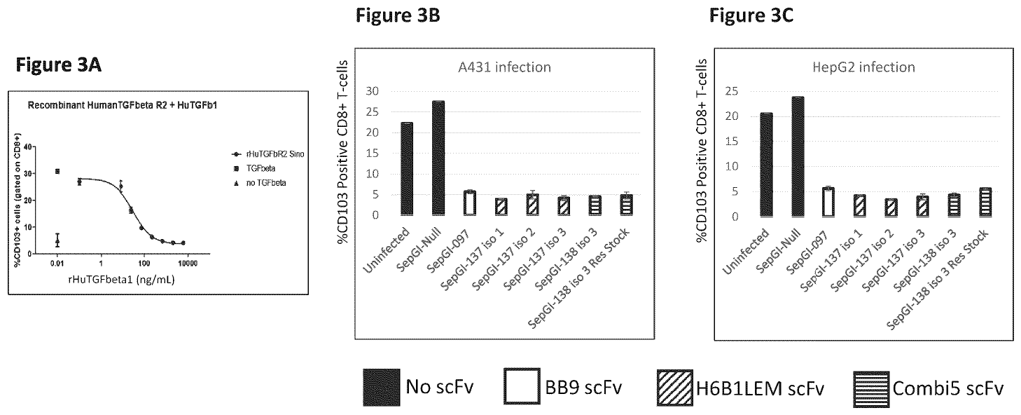

FIG. 3A provides a titration curve based on an expression assay in which increasing amounts of recombinant TGFβRII are able to prevent CD103 expression on CD8+ T cells stimulated with TGFβ1. FIG. 3B provides the results of the FACS analysis of CD8+ T cells treated with TGFβ1 and VFCMs of the SepGI-097, SepGI-137, and SepGI-138 HSVs that were produced in A431 cells and FIG. 3C provides the results of the FACS analysis of CD8+ T cells treated with TGFβ1 and VFCMs of the SepGI-097, SepGI-137, and SepGI-138 HSVs that were produced in HepG2 cells.

The results show that all of the viruses expressing an ScFv-Fc4-TGFβtrap were able to prevent expression of CD103 on the surface of CD8+ T cells. Host cells infected with either SepGI-097, encoding a “BB9” anti-PD-1 ScFv-Fc4-TGFβtrap fusion protein, SepGI-137, encoding an “H6B1LEM” anti-PD-L1 ScFv-Fc4-TGFβtrap fusion protein, or SepGI-138, encoding a “Combi5” anti-PD-L1 ScFv-Fc4-TGFβtrap fusion protein all produced viral supernatants that were able to block TGFβ signaling.

### Example 4. Blockade Assay for Anti-PD-1 ScFv and Anti-PD-L1 ScFv Function

The PD-1/PD-L1 blockade bioassay (Promega Corp., Madison, WI) was used to assess the function of the anti-PD-1 or anti-PD-L1 ScFv moiety of the ScFv-Fc-TGFβtrap viruses. This cell-based assay relies on the ability of PD-1/PD-L1 signaling to induce expression of genes regulated by the NFAT response element. Jurkat T cells that have been engineered to express a luciferase gene regulated by the NFAT response element and engineered effector CHO-K1 cells that express human PD-L1 as well as a protein that activates the T cell receptor are incubated together during the assay. Luciferase expression is not induced when CHO-K1 cells engage the Jurkat T cells via a PD-L1/PD-1 interaction; however, disruption of the interaction by an antagonist of PD-1/PD-L1 binding releases the inhibition, resulting in luciferase expression. The PD-1/PD-L1 blockade assays were performed essentially according to the manufacturer's instructions, where the test samples were dilutions of VFCMs from A431 cells infected with the engineered ScFv-Fc-TGFβtrap HSVs. VFCM of SepGI-Null, the HSV Seprehev® vector lacking an exogenous transgene, and conditioned medium from uninfected cells were also assayed as controls. Assays were performed in 96 well plates and the luciferase signal was analyzed with a Tecan (Mannedorf, Switzerland) Spark plate reader.

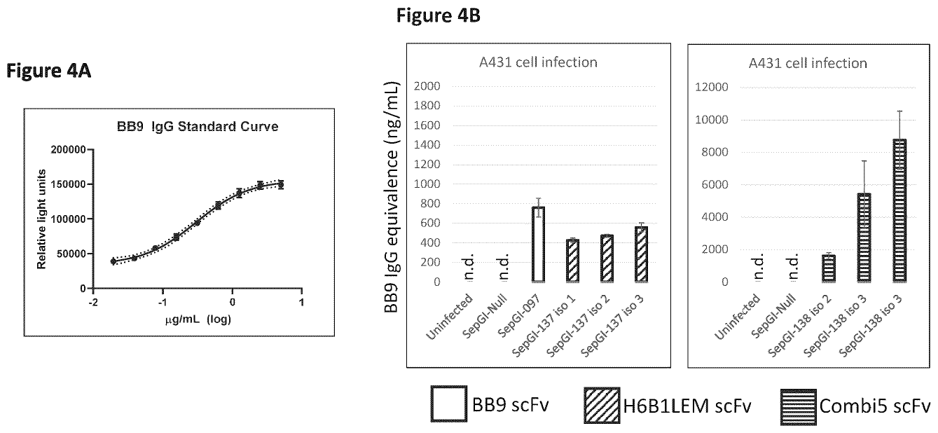

FIG. 4A is a titration curve for the PD-1/PD-L1 blockade assay using dilutions of the BB9 IgG, which binds PD-1, from 5 μg/ml to 0.02 μg/ml. FIG. 4B provides a graph of the results of the PD-1/PD-L1 blockade assay using VFCMs prepared from cultures of cells infected with SepGI-097, SepGI-137, and SepGI-138 HSVs, as well as the SepGI-Null vector as a control and conditioned media from uninfected cells as a further control, where the assay signal has been converted to μg/ml BB9 IgG equivalents. VFCMs produced from cells of infected with all three HSVs were able to block the PD-1/PD-L1 interaction to some degree, with the SepGI-138 virus demonstrating the highest degree of PD-1/PD-L1 blockade.

### Example 5. ScFv-Fc-TGFβTrap+IL12 Constructs

An additional series of constructs was produced in which a gene encoding IL12, operably linked to a separate promoter, was included along with the gene encoding a ScFv-Fc-TGFβtrap fusion protein (FIG. 1B). These dual gene constructs built on the ScFv-Fc-TGFβtrap fusion proteins of Example 1 that included, proceeding from the N-terminal end to the C-terminal end of the fusion proteins, a signal peptide (SEQ ID NO:34) a sequence encoding an ScFv antibody that specifically binds either PD-1 or PD-L1, a sequence encoding the human IgG4 Fc region (Fc4, SEQ ID NO:2), and a sequence encoding the extracellular domain of the TGFβ receptor II (TGFβRII ectodomain or TGFβRII_{ecto}, SEQ ID NO:7) by adding an expression cassette that included the Cytomegalovirus (CMV) promoter (SEQ ID NO:33) operably linked to a sequence encoding the p40 subunit of human IL12 (SEQ ID NO:57), followed by a sequence encoding a 2× elastin linker (SEQ ID NO:55) and then a sequence encoding the p35 subunit of human IL12 (SEQ ID NO:58). FIG. 1B shows schematically the bi-directional expression constructs which included the EF1α/HTLV hybrid promoter (SEQ ID NO:32) operably linked to the ScFv-Fc-TGFβtrap fusion protein-encoding gene and the CMV promoter operably linked to IL12.

In addition to constructs SepGI-123, SepGI-143, SepGI-158, SepGI-159, SepGI-144, and SepGI-160, that included the sequence of human IL12, the SepGI-162 and SepGI-167 constructs that encoded murine IL12 were produced to allow testing of the engineered viruses in mouse models (Table 3). Each of these constructs included a murine IL12 expression cassette comprising the CMV promoter (SEQ ID NO:33) operably linked to a sequence encoding the p40 subunit of murine IL12 (SEQ ID NO:59), followed by a sequence encoding a 2× elastin linker (SEQ ID NO) and then a sequence encoding the p35 subunit of murine IL12 (SEQ ID NO:60).

SepGI-123 was generated as a single gene HSV operably linked to the EF1α/HTLV promoter that included a gene encoding the human IL12 polypeptide (SEQ ID NO:51).

TABLE 3
ScFv-Fc-TGFβtrap + IL12 Constructs.

| HSV | | ScFv-Fc-TGFβtrap | |
|---|---|---|---|
| Strain | ScFv source | SEQ ID NO | IL12 gene |
| SepGI-123 | none | — | Human, |
| | | | SEQ ID NO: 51 |
| SepGI-143 | BB9 anti-PD-1 | 39 | Human, |
| | | | SEQ ID NO: 51 |
| SepGI-158 | RG1H10 | 41 | Human, |
| | anti-PD-1 | | SEQ ID NO: 51 |
| SepGI-163 | Pembrolizumab | 43 | Human, |
| | | | SEQ ID NO: 51 |
| SepGI-145 | Combi5 | 45 | Human, |
| | anti-PD-L1 | | SEQ ID NO: 51 |
| SepGI-144 | H6B1LEM | 47 | Human, |
| | anti-PD-L1 | | SEQ ID NO: 51 |
| SepGI-161 | Avelumab | 49 | Human, |
| | | | SEQ ID NO: 53 |
| SepGI-162 | BB9 anti-PD-1 | 39 | Murine, |
| | | | SEQ ID NO: 53 |
| SepGI-167 | Combi5 | 45 | Murine, |
| | anti-PD-L1 | | SEQ ID NO: 53 |

Dual gene constructs flanked by attL sites were generated by PCR cloning and cloned into an HSV1 Seprehvec® genome as described in Example 1. Following the recombination reaction, recombined viral genomic DNA was transfected into BHK (Baby Hamster Kidney fibroblast) cells and viruses were isolated as described in Example 1.

### Example 6. Quantitation of TGFβRII_{ecto} Produced by Recombinant Dual Gene Viruses Encoding an ScFv-Fc-TGFβTrap Fusion Protein and IL12

HSV strains SepGI-143 (BB9 anti-PD-1 ScFv-Fc4-TGFβRII_{ecto}), SepGI-144 (H6B1LEM anti-PD-L1 ScFv-Fc4-TGFβRII_{ecto}), and SepGI-145 (Combi5 anti-PD-L1 ScFv-Fc4-TGFβRII_{ecto}) were used to infect A431 cells and HepG2 cells for the production of VFCMs. SepGI-123, including a transgene encoding human IL12, but not including a construct for the production of a ScFv-Fc4-TGFβtrap fusion protein, was also used for the production of VFCM. Also included in the assay were the VFCM of cells infected with SepGI-Null and conditioned media of uninfected cells.

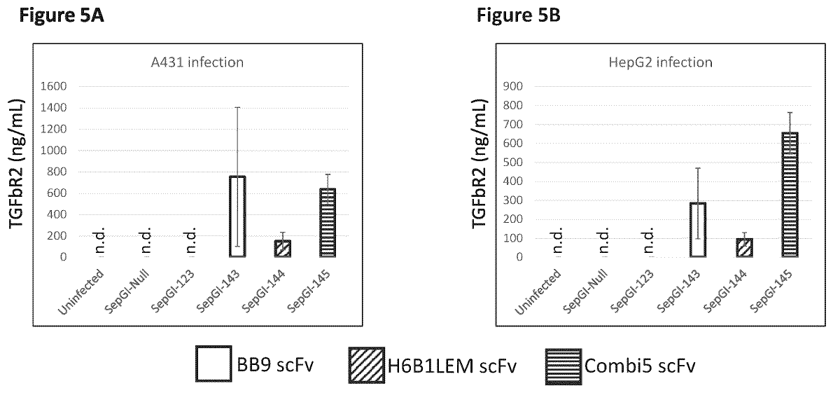

The VFCMs were tested for the amount of secreted ScFv-Fc-TGFβtrap in MSD assays as described in Example 2. FIG. 5A provides the results of the MSD assays using VFCMs of A431 cells infected with SepGI-Null, SepGI-123, SepGI-143, SepGI-144, and SepGI-145 and 5B and VFCMs of HepG2 cells infected with SepGI-Null, SepGI-123, SepGI-143, SepGI-144, and SepGI-145, respectively. Each of the dual gene HSVs produces detectable TGFβRII_{ecto}, with the HSV that included the Combi5 anti-PD-L1 ScFv-Fc4-TGFβRII_{ecto} (SepGI-145) expressing more of the fusion protein than either SepGI-143 (BB9 anti-PD-1 ScFv-Fc4-TGFβRII_{ecto}) or SepGI-144 (H6B1LEM anti-PD-L1 ScFv-Fc4-TGFβRII_{ecto}).

### Example 7. CD103 Cell-Based Assay for TGFβTrap Function for Dual Gene HSVs Expressing ScFv-Fc-TGFβTrap Plus IL12

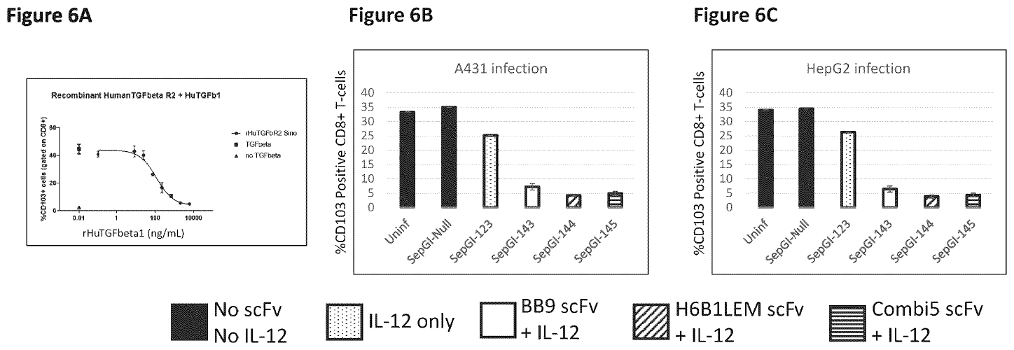

The CD103 assay described in Example 3 was used to assay the function of the ScFv-Fc-TGFβtrap fusion proteins in the VFCMs of cells infected with dual gene HSVs SepGI-143 (BB9 anti-PD-1 ScFv-Fc4-TGFβRII_{ecto}), SepGI-144 (H6B1LEM anti-PD-L1 ScFv-Fc4-TGFβRII_{ecto}), and SepGI-145 (Combi5 anti-PD-L1 ScFv-Fc4-TGFβRII_{ecto}). SepGI-Null (no transgenes) and Sep-123 (IL12 gene only) VFCMs were used as controls. FIG. 6A provides a titration curve based on the expression assay in which increasing amounts of recombinant TGFβRII are able to prevent CD103 expression on CD8+ T cells stimulated with TGFβ1. FIG. 6B provides the results from A431-produced VFCMs and FIG. 6C provides the results from HepG2-produced VFCMs. Culture medium from uninfected cells and cells infected with SepGI-Null demonstrate a base level of 30-35% CD103 expression by CD8+ T cells stimulated by TGFβ1 in this assay. Conditioned media from the SepGI-123 virus shows slight inhibition of the TGF-mediated signaling when compared with uninfected cells in this assay (approximately 28%) while each of the HSVs that included a gene encoding an ScFv-Fc-TGFβtrap shows strong inhibition of CD103 expression with respect to the controls of conditioned media of uninfected cells or of cells infected with the virus lacking a transgene (SepGI-Null). The viruses that included a transgene encoding an ScFv-Fc-TGFβtrap fusion protein all produced conditioned media that demonstrated at least 70% inhibition of CD103 expression when compared with controls, demonstrating that the ScFv-Fc-TGFβtrap fusion protein is produced by and secreted from the infected cells and has the desired inhibitory function with respect to TGFβ.

### Example 8. Blockade Assay for Anti-PD-1 ScFv and Anti-PD-L1 ScFv Function of Dual Gene Viruses

The PD-1/PD-L1 blockade bioassay (Promega Corp., Madison, WI) described in Example 4 was used to assess the function of the anti-PD-1 or anti-PD-L1 ScFv moiety of fusion proteins encoded by dual gene HSVs SepG1-143, SepGI-144, and SepGI-145. The PD-1/PD-L1 blockade assays were performed essentially according to the manufacturer's instructions, where the test samples were 0dilutions of VFCMs from A431 cells infected with the engineered ScFv-Fc-TGFβtrap HSVs. Assays were performed in 96 well plates and the luciferase signal was analyzed with a Tecan Spark plate reader.

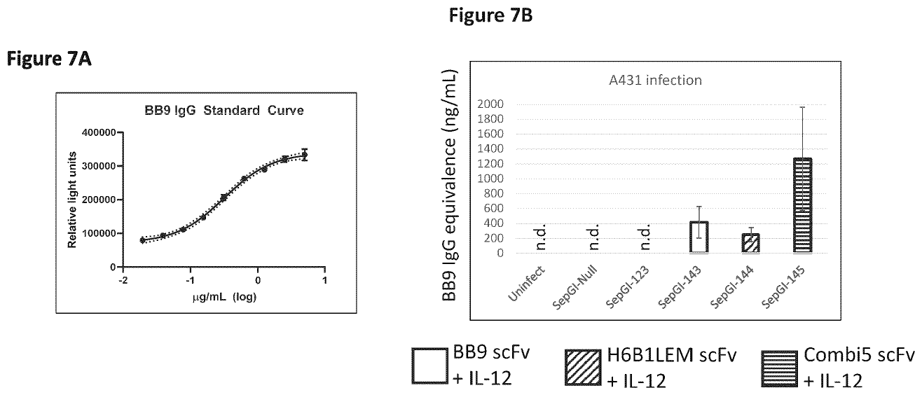

FIG. 7A is a standard curve PD-1 or PD-L1 binding activity for the PD-1/PD-L1 blockade assay using dilutions of the BB9 IgG, which binds PD-1, from 5 μg/ml to 0.02 μg/ml. FIG. 7B provides a graph of the results of the PD-1/PD-L1 blockade assay using VFCMs prepared from cultures of cells infected with SepGI-123 (lacking a gene encoding a ScFv-Fc-TGFβtrap), SepGI-143, SepGI-144, and SepGI-145 HSVs, where the assay signal (y axis) has been converted to μg/ml BB9 IgG equivalents. VFCMs produced from cells of all three dual gene HSVs were able to block the PD-1/PD-L1 interaction, with the SepGI-145 virus encoding the Combi5 anti-PD-L1 ScFv-Fc-TGFβtrap fusion protein demonstrating the highest degree of PD-1/PD-L1 blockade.

### Example 9. Functional Assay for Quantitation of IL12 Produced by Dual Gene Viruses

To quantitate the amount of IL12 produced by cells infected with the HSVs SepGI-123, SepGI-143, SepGI-144, and SepGI-145, a cell-based assay was used in which cells having a heterodimeric IL12 receptor and engineered to have a luciferase gene under the control of an IL12-responsive promoter (iLite® IL-12 Assay Ready Cells (Eagle Biosciences (Amherst, NH) catalog #BM4012) were incubated with lysates of cells infected with the recombinant HSVs. Promega Corporations One-Glo Luciferase system (catalog #E6120) was used for detection.

Briefly, the assay was performed by adding diluted VFCM from uninfected A431 cells or HepG2 cells (as controls) and from A431 cells and HepG2 cells infected with various HSVs to the wells of a 96 well plate. HSVs used to infect A431 cells or HepG2 cells and tested in the assay included SepGI-Null, SepGI-123, SepGI-143, SepGI-144, or SepGI-145. The lysates of infected cell cultures (VFCMs) were produced as described in Example 2. A dilution series of recombinant IL12 (R&D Systems, catalog #219-IL-005) was added to additional wells to generate a standard curve.

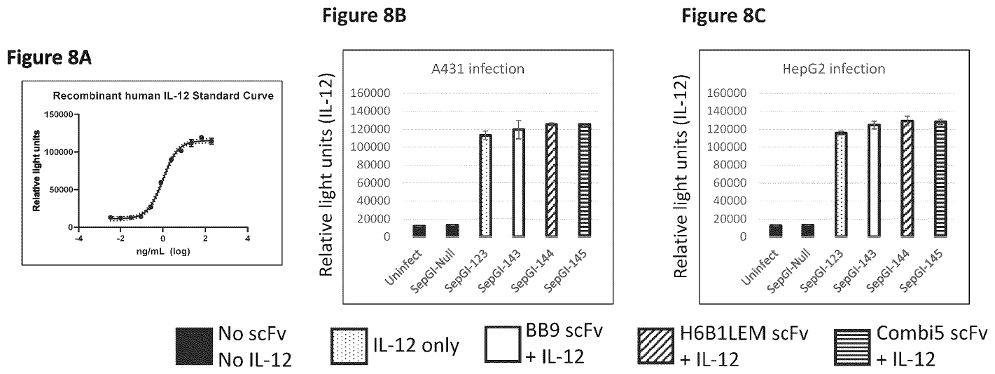

IL12 reporter cells were used essentially according to the manufacturer's instructions. Cells were thawed, diluted, and 40 μl added to each well of a 96-well plate. 40 μl of diluted VFCM was then added to the assay wells, the contents of the wells were mixed, and the plate was incubated for five hours at 37° C., 5% CO₂. The One-Glo luciferase reagent (Promega Corp., Madison, WI) was then added to each well (40 μL) and after 10 min at room temperature, firefly luciferase luminescence was measured using a Tecan Spark plate reader. The results are shown in FIG. 8. FIG. 8A provides the standard curve for recombinant IL-12 for relative luminescence units. FIG. 8B provides the luminescence from assays using uninfected A431 cell conditioned media, conditioned media from cells infected with a virus that did not include exogenous transgenes (SepGI-Null), and conditioned media from cells infected with the IL12 gene-containing viruses SepGI-123 (IL12 only), SepGI-143 (anti-PD-1 ScFv-Fc-TGFβtrap plus IL12), SepGI-144 (anti-PD-L1 ScFv-Fc-TGFβtrap plus IL12), or SepGI-145 (anti-PD-L1 ScFv-Fc-TGFβtrap plus IL12). FIG. 8C provides the luminescence from assays using uninfected HepG2 cell conditioned media, conditioned media from cells infected with a virus that did not include exogenous transgenes (SepGI-Null), and conditioned media from cells infected with the IL12 gene-containing viruses SepGI-123 (IL12 only), SepGI-143 (anti-PD-1 ScFv-Fc-TGFβtrap plus IL12), SepGI-144 (anti-PD-L1 ScFv-Fc-TGFβtrap plus IL12), or SepGI-145 (anti-PD-L1 ScFv-Fc-TGFβtrap plus IL12). The luminescence results show that all of the cell lysates produced from cells infected with IL12 gene-containing viruses produced IL12, whereas cells infected with viruses that did not include the IL12 gene did not show luminescence above that of uninfected control cells, demonstrating that the IL12 gene was efficiently expressed and secreted by the recombinant IL12 viruses SepGI-123, SepGI-143, and SepGI-145.

### Example 10. MSD Assay for TGFβTrap Produced by Dual Gene Virus SepGI-158

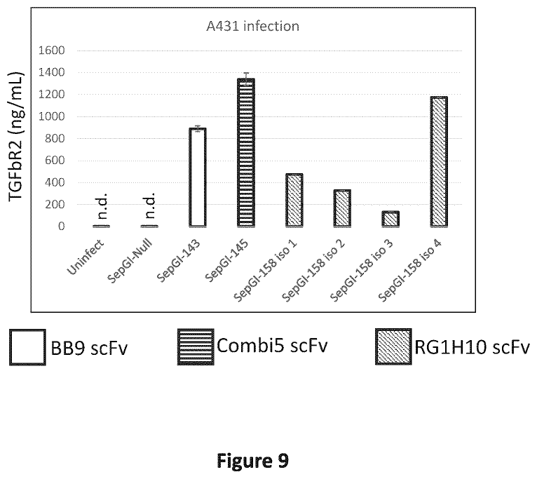

Cells infected with four different isolates of SepGI-158, a recombinant HSV that included a construct encoding an anti-PD-1 ScFv-Fc-TGFβtrap having an ScFv derived from monoclonal antibody R1GH10 and an IL12 transgene (SEQ ID NO:51), were tested for TGFβtrap production using the MSD assay described in Example 2. FIG. 9 shows the amount of TGFβtrap in the conditioned media of cells infected with recombinant HSVs SepGI-143 (encoding BB9 anti-PD-1 ScFv-Fc-TGFβtrap), SepGI-145 (encoding R1GH10 anti-PD-L1 ScFv-Fc-TGFβtrap), as well as the conditioned media of uninfected A431 cells and A431 cells infected with SepGI-Null. All of the SepGI-158-infected cells produced TGFβtrap, with isolate 4 producing the highest amount, which was comparable to the amount produced by cells infected with single gene virus SepGI-145.

### Example 11. Blockade Assay for Anti-PD-1 ScFv and Anti-PD-L1 ScFv Function of Dual Gene Virus SepGI-158

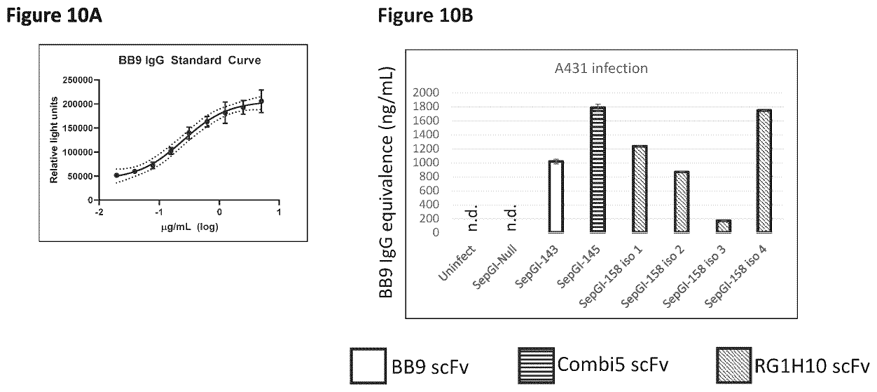

The virus-free conditioned media from SepGI-158 isolates 1-4 was also tested in the PD-1/PD-L1 blockade assay described in Example 4 to assess the amount of PD-L1-binding activity produced by SepGI-158 infected cells. The assay, in which conditioned media from the same four isolates of SepGI-158 analyzed for TGFβtrap in Example 10, also compared the amounts of TGFβtrap produced by cells infected with recombinant HSVs SepGI-143 (encoding BB9 anti-PD-1 ScFv-Fc-TGFβtrap), SepGI-145 (encoding Combi5 anti-PD-L1 ScFv-Fc-TGFβtrap), as well as the conditioned media of uninfected A431 cells and A431 cells infected with SepGI-Null. FIG. 10 shows cells infected with all of the SepGI-158 virus isolates produced ScFv-Fc-TGFβtrap proteins with PD-1/PD-L1 blocking activity. Consistent with the amounts of TGFβtrap produced by the SepGI-158-infected cells (Example 10), isolate 4 produced the highest amount of PD-L1 blocking activity, which was comparable to the amount produced by cells infected with SepGI-145.

### Example 12. IL12 Assay of Dual Gene Virus SepGI-162

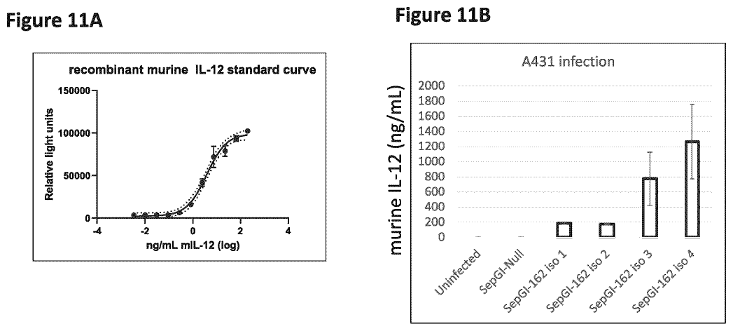

A431 cells infected with four separate isolates of SepGI-162, which included a construct encoding the BB9 anti-PD-1 ScFv-Fc-TGFβtrap (SEQ ID NO:39) and the murine IL12 gene (SEQ ID NO:53) were used for producing VFCM to test for the production of IL12 using the assay described in Example 9. Murine IL12 is able to interact with the human IL12 receptor and cause signal transduction as measured in the assay. FIG. 11A provides the standard curve for recombinant murine IL-12 (Invivogen, San Diego, CA, catalog #rcyc-mil12) for relative luminescence units. When the IL12 assay was re-calibrated based on the standard curve of murine IL12, the amount of murine IL12 produced by SepGI-162 was approximately 1 μg/ml (FIG. 11B).

### Example 13. Replication of SepGI-145 in Murine and Canine Cells

The replication of the oncolytic HSV SepGI-145 (encoding the Combi5 anti-PD-L1 ScFv-Fc4-TGFβRII_{ecto} fusion protein plus human IL12) was assessed in mouse embryonic cell line 3T6 and in canine kidney primary fibroblast (cKPF) cells isolated from fresh kidney tissue of a normal beagle dog (passage 1). Vero cells, which are known to support replication of both non-attenuated HSV strains and HSV strains that are functionally deleted for the RL1 gene, were used as host cells for comparison.

In the first experiment, SepGI-145 and three control I-ISVs were used to inoculate 3T6 cells and Vero cells. The control viruses were SepGI-Null, which had the same backbone as SepGI-145 (functionally deleted in the RL1 gene) but lacked transgenes and HSV1716, also called “Seprehvir®”, which is very similar to Seprehvec® but has a 755 bp deletion rather that a 695 bp deletion in both copies of the RL1 gene. Seprehvir® has been used in numerous trials (e.g., Rampling et al. (2000) Gene Ther. 7:859-66; Harrow et al. (2004) Gene Ther. 11:1648-1668; Streby et al. (2017) Clin. Cancer Res. 23:3566-3574; Streby et al. (2019) Mol. Ther. 27:1930-1938) “Virttu 17+” is the HSV 17+ strain that is the progenitor HSV-1 strain for RL1 deletion strains Seprehvir® and Seprchvec® (MacLean el al. (1991) J. Gen. Virol. 72:631-639); it has fully functional RL1 genes.

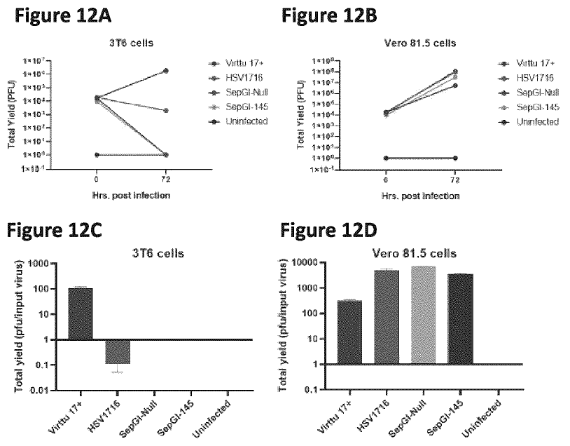

To test replication on murine 3T6 cells, wells of 6 well plates were seeded with 2×10⁵ cells and incubated overnight at 37° C., 5% CO₂. The following day, the 3T6 cells and Vero cells were infected with either Virttu 17+, Seprehvir, SepGI-Null, or SepGI-145 at MOI 0.1. The cells were incubated with virus for 1 hour, after which the supernatants of the wells (To supernatants) were collected and frozen and fresh culture medium was added. The cultures were then incubated for 72 hours, at the end of which the 72 hr supernatants were collected and frozen. The two time point supernatants (0 hr, or pre-replication, and 72 hr. post replication) were then used to titer on Vero cells. The results are seen in FIG. 12. The recombinant viruses lacking a functional RL1 gene, namely, HSV1716, SepGI-Null. and SepGI-145, did not replicate in 3T6 cells; only “Virttu 17+” demonstrated replication in this embryonic mouse cell line (FIGS. 12A and 12C). On the other hand, all viruses, regardless of whether they had a functional RL1 gene, were able to replicate in Vero cells (FIGS. 12B and 121).

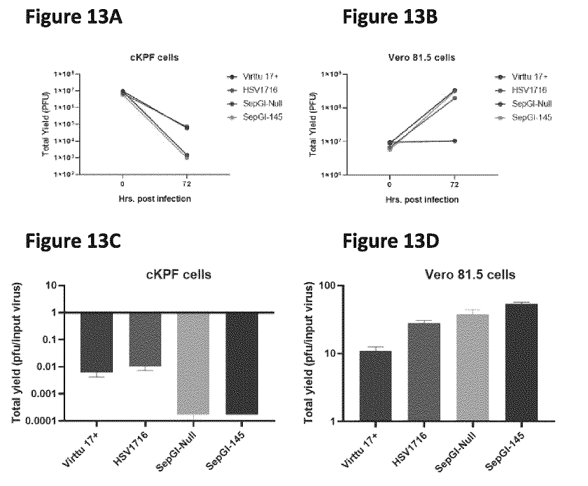

The same experiment was performed using canine primary kidney primary fibroblast (cKPF) cells and Vero cells, except that 9×10⁴ cells were seeded into wells of 6 well plates and incubated overnight at 37° C., 5% CO₂ prior to the assay. In this case, none of the HSVs tested replicated in the cKPF cells (FIGS. 13A and 13C), while all of the HSVs replicated in Vero cells (FIGS. 13B and 13D). These results demonstrate that although RL1-negative mutants such as HSV1716 (Seprehvir®). SepGI-Null, and SepGI-145 are able to replicate to high titers on Vero cells, they fail to replicate in mouse embryo fibroblast (3T6) and the primary canine cKPF cells.

### Example 14. Efficacy of Anti-PD-1 ScFv-FcTGFβTrap+IL12 HSV SepGI-162 in a Bladder Cancer Xenograft Model in Mice

An in vivo study to test the effect of a dual gene HSV that included a BB9 anti-PD-1 ScFv-Fc4-TGFβtrap gene and a murine IL12 gene (SepGI-162) as described in the previous example was performed on C57BL/6 mice implanted with syngeneic MB49 tumors. MB49 cells are murine urothelial carcinoma cells that were derived by culturing MB49 primary bladder cells in the presence of 7,12-dimethylbenz[a]anthracene. The BB9 antibody specifically binds both human and mouse PD-1, and the amino acid sequence of mature human TGFβ1 is 99% identical with the amino acid sequence of mature mouse TGFβ1.

On day 0, MB49 cells (1×10⁵ cells) were implanted subcutaneously in the right flanks of seven week old female mice and allowed to form tumors. Each treatment group included eight mice. The SepGI-162 treatment group was treated with the SepGI-162 virus: Seprehvec® including the BB9 anti-PD-1 ScFv-Fc-TGFβtrap gene (SEQ ID NO:39) plus the murine IL12 gene (SEQ ID NO:53)) (Table 2).

Treatment with virus or the formulation buffer control began at day 8 and was repeated every other day or, if a treatment day fell in the weekend, the treatment was given on the next weekday, for a total of nine doses. The mice were weighed once a week and the tumors were measured with calipers twice per week. 1×10⁷ pfu of HSV in 50 μl formulation buffer was administered by peritumoral injection. Mice that had a loss bodyweight equal to 15% of the body weight at day zero or whose tumor size exceeded 2000 mm³ were euthanized.

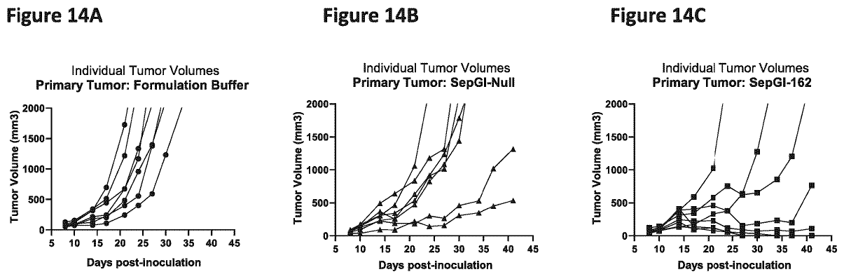

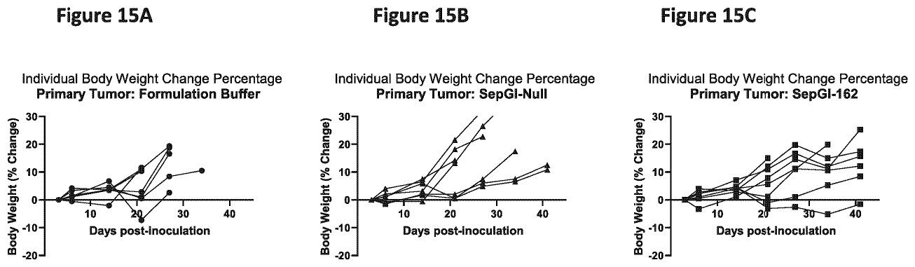

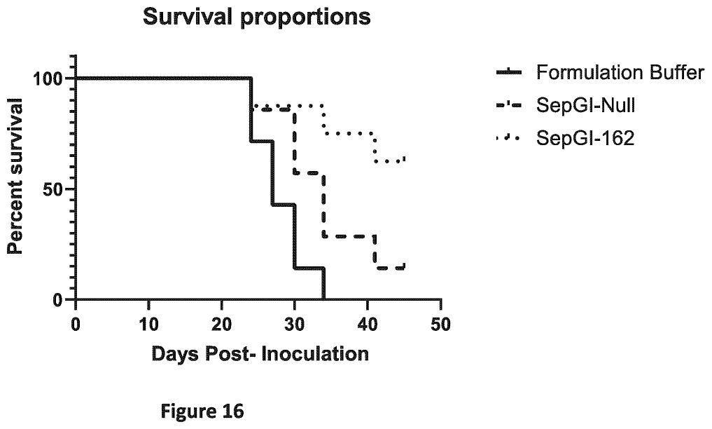

FIGS. 14A-C provide the tumor volumes of the individual mice in the formulation buffer control group, the SepGI-Null treatment group, and the SepGI-162 treatment group, and FIGS. 15A-C show the body weights of the mice over the course of the study. For all mice in the formulation buffer control group (FIG. 14A), tumor volume increased over the course of the study, with the rate of tumor size increasing after two weeks. Two of the SepGI-Null-treated mice experienced a reduced rate of tumor volume increase (FIG. 14B). Dramatic results were seen in the SepGI-162 treatment group (FIG. 14C). While all but one of the Formulation Buffer control group had tumors of 2000 mm³ by day 30, only two of the SepGI-162-treated mice had tumors of that volume by day 30. Three of the SepGI-162-treated mice were completely cured of the tumor, and tumor growth was delayed or suppressed in an additional two mice with respect to mice of the control group. The efficacy of the SepGI-162 treatment is demonstrated in the survivorship graph of FIG. 16, where tumor size of 2000 mm³, resulting in euthanasia, was recorded as death. The survivorship graph illustrates that none of the Formulation buffer control group survived past day 34, whereas at the same time point the survivorship of the SepGI-Null-treated group was 55% and the SepGI-162-treated group was 90%. Further, at the end of the study (45 days) 65% of the SepGI-162-treated group was alive. The effects of SepGI-162 treatment on the tumor were highly significant at the p<0.005 level (Table 4).

TABLE 4
Log-rank (Mantel-Cox) Test: Survival Curve Comparison.

| Treatment vs. | Treatment | Significance | P-value |
|---|---|---|---|
| Formulation Buffer | SepGI-Null | ns | 0.0524 |
| Formulation Buffer | SepGI-162 | ** | 0.0022 |

### Example 15. Re-Challenge of SepGI-162-Treated Mice with MB49 Bladder Cancer Cells

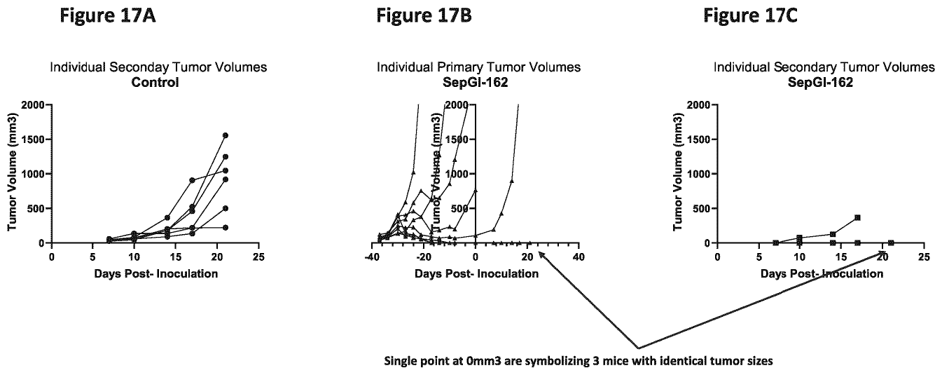

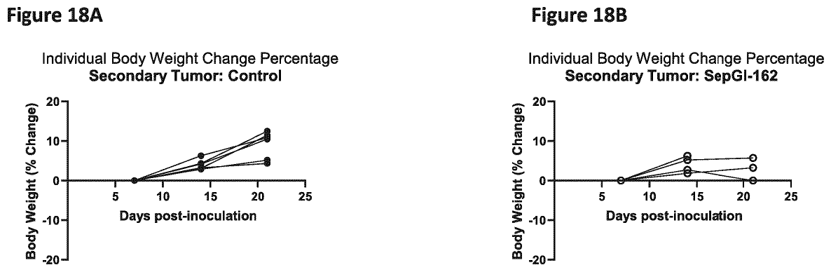

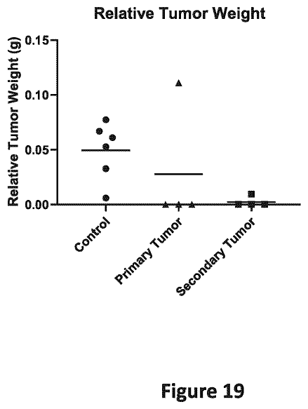

The success of treatment with dual gene HSV SepGI-162 in treating the syngeneic mouse bladder cancer model detailed in Example 14, above, allowed for a tumor re-challenge experiment. The three mice that were cured of the MB49 tumor as well as the mouse exhibiting no tumor growth by the end of the study at day 42 were injected with a further inoculum of MB49 tumor cells (1×10⁵ cells in 100 μl volume) on the opposing flank to where the original tumor cells were inoculated. No further treatment with virus was performed as the study was designed to observe any protective effects of prior viral treatment on progression of a later-presenting (secondary) tumor as well as on the original (primary) tumor. Tumor volume at the secondary and primary inoculation sites was assessed based on measurements every three days (FIG. 17) and body weights were recorded (FIG. 18). Tumors were dissected and weighed when mice were euthanized at the completion of the study or when the mice were euthanized for other reasons (FIG. 19).

FIG. 17A shows a control in which six mice not previously inoculated with tumor were injected with the same number of cells as the re-challenge mice. These control mice were aged-matched with the re-challenge mice and inoculated with the same cells that were used to inoculate the re-challenge mice and were not treated with any oncovirus formulation. Tumors were established in all control mice. FIGS. 17B and 17C show the growth of primary and secondary tumors respectively in the re-challenge mice. The lower data points in both FIG. 17B and FIG. 17C each represent three individual mice that exhibited no tumor growth of the primary tumor (FIG. 17B) and no establishment of a secondary tumor from the re-challenge tumor cells (FIG. 17C). One mouse that had delayed tumor growth in the original experiment exhibited growth of the primary and secondary tumors upon re-challenge. None of the mice exhibited body weight loss (FIGS. 18A and 18B). Tumor failed to become established in the three mice cured of the primary tumor, indicating a durable anti-tumor immune response by the mice treated with the dual gene SepGI-162 HSV expressing anti-PD-1ScFc-Fc4-TGFβtrap and murine IL12. FIG. 19 provides the endpoint tumor sizes of control mice and mice previously treated with SepGI-162 normalized with respect to the number of days post-inoculation at which the tumors were dissected. The figure illustrates the inhibitory effect of treatment with the SepGI-162 virus on both primary and subsequent secondary tumor growth.

### Example 16. Cytotoxicity of HSVs on Canine Osteosarcoma Cell Lines

Cells of the canine osteosarcoma cell lines OSCA-40 were infected with the Seprehvec viruses SepGI-145, SepGI-Null, and SepGI-dsred (Seprehvec® including a Ds red gene operably linked to the CMV promoter inserted into the internally deleted RL1 gene loci) to determine the cytotoxic effects of these HSVs on the canine tumor cells.

For these experiments, OSCA-40 cells were seeded into the wells of 96 well E-plates (xCELLigence® cell analyzer, Acea Biosciences, San Diego, CA) at 10,000 cells/well, allowed to settle overnight at 37° C., 5% CO₂, and then were infected at MOI 0.1, MOI 1.0, and MOI 10.0 with the SepGI-145, SepGI-Null, and SepGI-dsred viruses. Triplicate assays were performed for each MOI with each of the viruses.

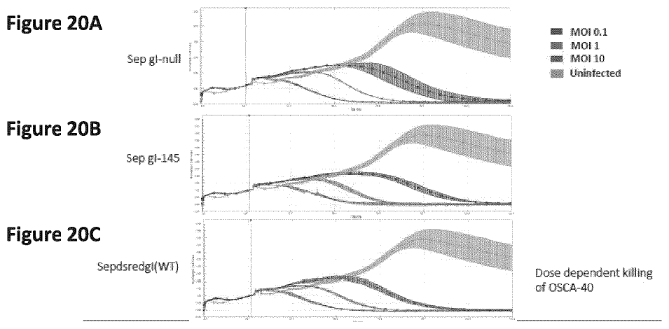

Proliferation was monitored in real time over an additional 4.5 days using the xCELLigence® Real Time Cell Analyzer (Acea Biosciences) essentially according to the manufacturer's instructions. Proliferation curves of the OSCA-40 cells infected with SepGI-Null, SepGI-145, and SepGI-dsred, respectively are provided in FIG. 20A-C where the normalized cell index (y axis) is proportional to the number of cells in the wells. FIG. 20 shows that OSCA-40 cells are sensitive to all three HSVs, demonstrating dose-dependent (MOI-dependent) inhibition of proliferation with respect to uninfected cells.

### Example 17. Functionality of TGFβRII_{ecto} Expressed in SepGI-145 Infected OSCA-40 Canine Osteosarcoma Cells

To test whether SepGI-145-infected cells produced and secreted functional anti-PD-L1-Fc-TGFβRII fusion protein, the cells of the canine osteosarcoma cell line OSCA-40 were infected with either SepGI-145 or SepGI-Null in the wells of a 12 well plate (1×10⁵ cells per well) at MOI 1.0. The cells were incubated with the HSV strains for 72 hours at 37° C., 5% CO₂. Supernatants were then removed from the wells and were separately filtered through 0.1 μm membranes (Pall Acrodisc Syringe filter part #4611) to remove virus. The post-viral culture supernatants (VFCMs) were then aliquoted and stored at 4° C. for up to two days or placed at −80° C. for longer storage.

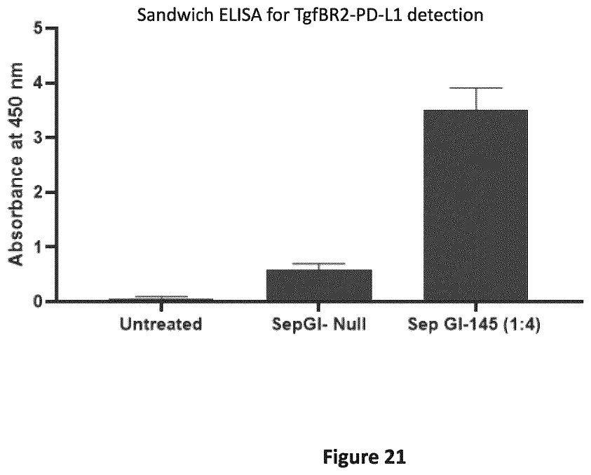

For analysis of the anti-PD-L1-Fe-TGFβRII fusion protein, a sandwich ELISA assay was performed using canine TGFβ1-coated plates (20 ng/well) (Sino Biological #70087-D08H). Control wells were coated with 20 ng human TGFβ1 (BioLegend catalog #580702). Coating of plates with canine or human TGFβ1 was performed at 4° C. overnight, after which wells were washed with PBS/0.01% Tween20, then blocked with PBS/I % FBS for 2 hours at room temperature and then washed again (PBS/0.01% Tween20). Supernatants of infected cells were added to the coated wells and binding was performed at room temperature for 2 h. The wells were then washed and recombinant human PD-L1-Fe (R&D #156-B7) was added to the wells (10 ng/well) and allowed to bind for 2 h at room temperature, after which the wells were again washed before adding the biotin anti-human Ig-Fc (BioLegend #409307) followed by 1 hr incubation at room temperature. Avidin-HRP reagent was then added into the wells for 30 min incubation at room temperature, the plate was then washed three times, and then 100 μl HRP substrate solution was added to each well and the plate was incubated for 20 min at room temperature in the dark. Stop solution (100 μl) was then added and the plates were read at 450 nm. FIG. 21 shows the results of the ELISA designed to detect functional (TGFβ-binding) TGFβRII_{ecto} and functional (PD-L1-binding) anti-PD-L1 antibody which are produced as part of the SepGI-145 anti-PD-L1-Fe-TGFβRII fusion protein by capture using TGFβ and detection using PD-L1. The supernatants of untreated (uninfected) cells and cells infected with SepGI-Null showed little to no absorbance, whereas at a 1:4 dilution, the supernatant (VFCM) of SepGI-145 infected cells showed production of the PD-L1-Fc-TGFβRII fusion protein indicating that the fusion protein binds to canine TGFβ.

### Example 18. Blockade Assay for Anti-PD-L1 ScFv Function of Anti-PD-L1-Fc-TGFβTrap Fusion Protein

The PD-1/PD-L1 blockade bioassay (Promega Corp., Madison, WI) described in Example 4 was used to assess the function of the anti-PD-L1 ScFv moiety of the Combi5 anti-PD-L1 ScFv-Fc-TGFβtrap fusion protein encoded by SepGI-145. The test samples were concentrated VFCMs from OSCA-40 cells infected with either SepGI-145 or SepGI-Null. Assays were performed in 96 well plates and the luciferase signal was analyzed with a Tecan (Männedorf, Switzerland) Spark plate reader.

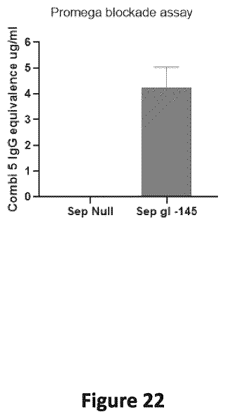

FIG. 22 provides a graph of the results of the PD-1/PD-L1 blockade assay using VFCMs prepared from cultures of cells infected with SepGI-Null and SepGI-145, where the assay signal has been converted to μg/ml Combi5 anti-PD-L1 IgG equivalents. VFCM produced from cells infected with the SepGI-145 HSV, but not VFCM of cells infected with SepGI-Null, was able to block the PD-1/PD-L1 interaction.

### Example 19. Assay of IL12 Activity of SepGI-145 Infected Cells

To quantitate the amount of IL12 produced by cells infected with SepGI-145, the cell-based assay of Example 8 was used in which cells having a heterodimeric IL12 receptor and engineered to have a luciferase gene under the control of an IL12-responsive promoter (iLite® IL-12 Assay Ready Cells (Eagle Biosciences (Amherst, NH) catalog #BM4012) were incubated with lysates of cells infected with the recombinant HSVs. Promega Corporations One-Glo Luciferase system (catalog #E6120) was used for detection.

The assay was performed by adding diluted VFCMs from OSCA-40 cells infected with either SepGI-145, or, as a control, SepGI-Null, which lacks a transgene to the wells of a 96 well plate. A dilution series of recombinant human IL12 (R&D Systems, catalog #219-IL-005) was added to additional wells to generate a standard curve.

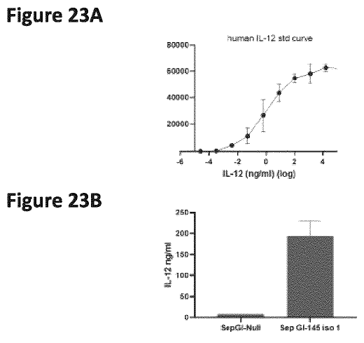

IL12 reporter cells were used essentially according to the manufacturer's instructions. Cells were thawed, diluted, and 40 μl (5×10⁴ cells) were added to each well of a 96-well plate. 40 μl of diluted VFCM was then added to the assay wells, the contents of the wells were mixed, and the plate was incubated for five hours at 37° C., 5% CO₂. The One-Glo luciferase reagent (Promega Corp., Madison, WI) was then added to each well (40 μL) and after 5 min at room temperature, firefly luciferase luminescence was measured using a Tecan Spark plate reader. The results are shown in FIG. 23. FIG. 23A provides the standard curve for recombinant IL12 for relative luminescence units. FIG. 23B provides the luminescence from assays using VFCM from cells infected with a virus that did not include exogenous transgenes (SepGI-Null), and VCFM from cells infected with the IL12 gene-containing virus SepGI-145 (anti-PD-L1 ScFv-Fc-TGFβtrap plus IL12). The luminescence results show that the cell lysates produced from cells infected with IL12 gene-containing SepGI-145 virus included IL12, whereas cells infected with viruses that did not include the IL12 gene did not show luminescence above that of uninfected control cells, demonstrating that the IL12 gene was efficiently expressed and secreted by the recombinant IL12 virus SepGI-145.

### Example 20. Safety Study of Seprehvir® (HSV1716), SepGI-145, and SepGI-Null in Canines

A three-pail study was performed on healthy adult beagle dogs to assess the safety of herpes viruses derived from HSV-1 strain 17+ that are functionally deleted for the RL1 gene. The study used four groups of dogs. Groups 1, 2, 3, and C (control), each of which included two male and two female dogs. All dogs enrolled in the study were found to test negative for antibodies to herpes virus prior to the onset of the study. The study assessed the dogs for any adverse effects of the injected herpes virus, for viral shedding, and for dog-to-dog transfer of the virus. In Part I of the study Group 1 dogs were injected with HSV1716, in Part II of the study Group 2 dogs were injected with SepGI-145, and in Part III of the study Group 3 dogs were injected with SepGI-Null. Dogs of Group C, the nontreatment group, were used as controls in all three parts of the study, which were conducted sequentially, and also served as sentinels for detecting any dog-to-dog transfer of the virus.

TABLE 5
HSV Safety Study, Beagle Dogs.

| | | Number of | | | |
|---|---|---|---|---|---|
| | | Animals | Dose Level | Study | IV Dose |
| Study | Treatment | Treated | (pfu/ml) | Days | Volume |
| Part I | HSV1716 | 2M/2F | 1 × 10⁸ | 0 | 1 mL |
| Part II | SepGI-145 | 2M/2F | 1 × 10⁸ | 0, 28 | 1 mL |
| Part III | SepGI-Null | 2M/2F | 1 × 10⁸ | 28 | 1 mL |

In Part I of the study which assessed the safety of Seprehvir® (HSV1716), Group C (control) dogs and Group 1 (HSV1716 treatment) dogs were co-mingled for seven days prior to the onset of the study on Phase I Day 0. On Day 0 of the Part I study, Group 1 dogs received an injection of HSV1716 and Group C (control) dogs received an injection of formulation buffer (Hartmann's solution containing 10% glycerol). The Group C and Group 1 dogs were co-mingled for an additional 21 days post-treatment (until Part I Day 21). Group 1 and control dogs had physical examinations at the beginning and end of the study (Part I Days 0 and 21). During the Part I study weekly blood samples were taken from Group 1 dogs and control dogs for assessing blood chemistry and hematology, and to test for antibodies to the virus (serology). In addition, urine, fecal, saliva, and nasal swab samples were taken from Group 1 and control dogs on Days 0, 3, 5, 7, 9, and 14 to test for any viral shedding (Table 6).

TABLE 6
Test Samples from Dogs in Three-Part Safety Study.

| | | | Urine Sample; | |
|---|---|---|---|---|
| | Injection | Blood | Fecal, Salivary, | Physical |
| | Injection | Sampling | Nasal Swab | Examination |
| Study | Days | Study Days | Study Days | Study Days |
| Part I | 0 | 0, 7, 14, 21 | 0, 3, 5, 7, 9, 14 | 0, 21 |
| | | (serology only) | | |
| Part II | 0, 28 | 0, 7, 14, 21 | 0, 3, 5, 7, 9, 14 | 0, 21 |
| | | (serology only) | | |
| | | 28, 35, 42, 49 | 28, 31, 33, 35, | 28, 49 |
| | | (serology only) | 37, 42 | |
| Part III | 0 | 0, 7, 14, 21 | 0, 3, 5, 7, 9, 14 | 0, 21 |
| | | (serology only) | | |

In Part II of the study, which assessed the safety of SepGI-145, Group 2 (SepGI-145 treatment), Group 2 and control dogs were co-mingled for one week prior to the onset of treatments (i.e., from day −6 to Day 0 of the study). Group 2 dogs received two doses of the virus and control dogs received two doses of formulation buffer, the first dose on day 0 of the Part II study and the second dose four weeks later, on Day 28. Because SepGI-145 was engineered for expression of a fusion protein, a two dose study was designed to allow for observation of a potential immunological response to the second dose of virus. Control dogs and Group 2 dogs continued to be co-mingled until three weeks after the second treatment. During the course of the Part II study, Group 2 and Control dogs received physical examinations on treatments days and again three weeks later (i.e., on Part II Days 0 and 21, and on Days 28 and 49). Blood samples were taken weekly beginning on the day of treatment to test blood chemistry, hematology, and for virus antibodies. Urine, fecal, saliva, and nasal samples were taken on days throughout the study and tested for the presence of virus (Table 6).

In Part III of the study, Group 3 dogs and control Group dogs were comingled for one week prior to the onset of treatment with the SepGI-Null virus (Group 3) or formulation buffer (control group C) on Day 0 of the study. The dogs continued to be co-mingled for three weeks after the treatment. During the Part Ill study weekly blood samples were taken from Group 3 dogs and control dogs for assessing blood chemistry and hematology, and to test for antibodies to the virus (serology). In addition, urine, fecal, saliva, and nasal swab samples were taken from Group 3 and control dogs on Days 0, 3, 5, 7, 9, and 14 to test for any viral shedding (Table 6).

The animals were housed in accordance with the standards and regulations set forth in the Animal Welfare Act and Animal Welfare Regulations of Jan. 1, 2017 and were observed twice daily for general well-being, appearance, and behavior from the beginning of acclimation through the end of the study. Scheduled physical examinations were performed by a licensed veterinarian prior to treatment administration on Study Days 0 and 21 (Groups 1 and 2). Study Days 28 and 49 (Groups 1 and 3), and Study Days 56 and 77 (Groups 1, 3 and 4). One dog in Group 3 was removed from the study on Day 61 and euthanized due to injuries incurred in a dog fight.

Serum from blood samples was tested for neutralizing antibodies to Seprehvir (HSV1716). All dogs were seronegative for HSV1716 before infusion with virus and all dogs tested at various timepoints after infusion with virus as well as control Group 1 dogs remained seronegative. Blood samples were collected for blood chemistry, hematology, and serology (antibody) testing and urine, fecal, salivary, and nasal samples were collected to test for the presence of the virus during the study according to the schedule shown in Table 6.

Samples collected during the study at different time points after infusions were evaluated on Vero cells for the shedding of oncolytic herpes virus. Although a few samples resulted in morphological changes in the Vero cells when applied to the cultures, none of the samples showed a herpes virus-specific cytopathogenic effect in the second passage. The data showed that intravenous infusion of RL1-deleted oncolytic herpes viruses in dogs did not result in shedding of the virus in urine, saliva, nasal swabs and fecal swab samples collected at different time points.

All animals were humanely euthanized following the last sample collection of their respective study phase according to facility SOPs. At Study Day 21 (Group 1) and Study Day 77 (Groups 2, 3, and C), following euthanasia, tissue samples were dissected and tested for the presence of the virus or any abnormalities. No virus was detected in any dissected tissues and no abnormalities were found that were attributable to the virus. It was concluded that the HSV1716 virus and related viruses SepGI-145 and SepGI-Null do not replicate in normal canine cells.

### Example 21. Preparation of Concentrated VFCM from HSVs SepGI-097, SepGI-138, and SepGI-162

Virus-free conditioned media (VCFM) was produced from a virus expressing a BB9 anti-PD-1 ScFv-Fc-TGFβtrap fusion protein (SepGI-097), a virus expressing a Combi5 anti-PD-L1 ScFv-Fc-TGFβtrap fusion protein (SepGI-138), a virus expressing a BB9 anti-PD-1 ScFv-Fc-TGFβtrap fusion protein as well as murine IL12 (SepGI-162), and a virus expressing a Combi5 anti-PD-L1 ScFv-Fc-TGFβtrap fusion protein as well as murine IL12 (SepGI-167). VFCM was also made from SepGI-Null (Seprehvec lacking a transgene) for use as a control.

Supernatants of infected cells were prepared by infecting BHK cells. BHK cells were seeded into 850 cm² roller bottles by dissociating BHK cells grown to 100% confluency in a T225 flask and resuspending the cells to a final volume of 12 ml culture medium (DMEM/F12+10% FBS+5% TPB). Three ml of the cell suspension was added to each roller bottle. The inoculated bottles were incubated for approximately three days at 37° C. (until ˜90% confluent), with an estimated cell number of 8×10⁷ cells per roller bottle. The roller bottles were inoculated with 4×10⁷ pfu of either SepGI-Null, SepGI-097. SepGI-138, SepGI-162, or SepGI-167 virus. For infection of roller bottles, media was removed from the confluent cells layer and 25 ml of fresh culture media including the viral inoculum was added to the bottles. The bottles were then incubated at 37° C. for three days. At the end of the infection period, the supernatants were removed from the bottle and flask and transferred to 50 ml conical tubes which were spun at 2,095 rcf for 15 min. The supernatants were transferred to new 50 ml conical tubes and the centrifugation was repeated.

The final supernatants were successively filtered through a 0.8 micron cellulose acetate syringe filter, a 0.22 micron cellulose acetate syringe filter, and an acrodisc 0.1 micron filter to remove virus. The VFCMs were then stored overnight at 4° C.

The VFCMs were then concentrated using two Amicon Ultra-4 concentrators (30 kDa MW cutoff. Millipore #UFC803024) per 25 ml VFCM to reduce the volume to approximately 660 μl per VFCM. The concentrated VFCM was then mixed well, aliquoted, and stored at −80 C.

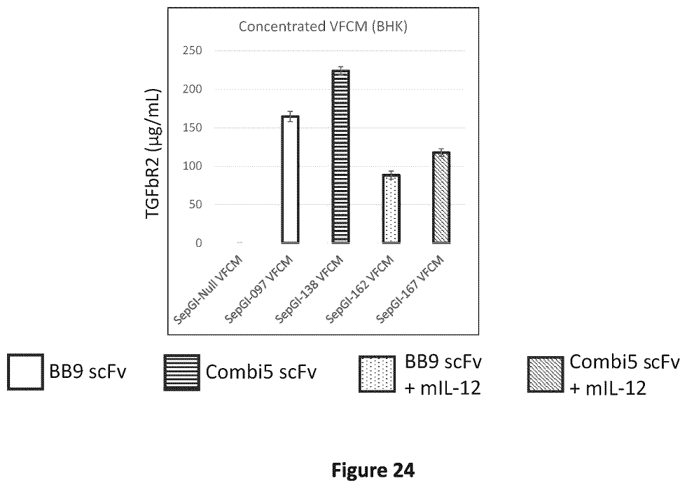

FIG. 24 provides the results of analyzing the concentrated VFCMs of SepGI-Null, SepGI-138, SepGI-162, and SepGI-167 infected cells for TGFβtrap content. All of the post-viral supernatants contained TGFβtrap at a concentration of 100-200 μg/ml. No TGFβtrap was detected in the post-viral supernatants of cells infected with the SepGI-Null control virus.

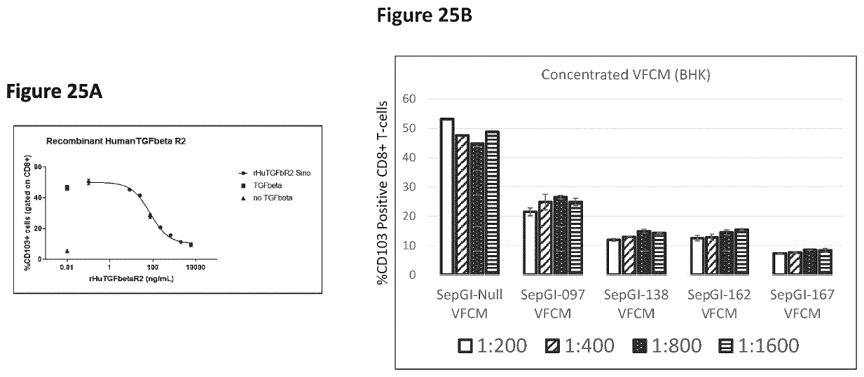

FIG. 25 provides the results of analyzing the concentrated VFCMs of SepGI-Null, SepGI-138, SepGI-162, and SepGI-167 infected cells for TGFβtrap function using the cell-based CD103 assay as detailed in Example 3. All of the concentrated VFCMs from cells infected with viruses that included a gene for expressing a ScFv-Fc-TGFβtrap fusion protein (SepGI-097, SepGI-138, SepGI-162, and SepGI-167) were able to inhibit TGFβ signaling in this assay, at dilutions up to 1:1600.

### Example 22. Dosage Study of SepGI-162 (Anti-PD-1 ScFv-FcTGFβTrap+IL12) the MB49 Bladder Cancer Xenograft Model in Mice

An in vivo study was performed to investigate the effect of different dosing regimens of dual gene HSV SepGI-162 that included a BB9 anti-PD-1 ScFv-Fc4-TGFβtrap gene and a murine IL12 gene, as described in the previous examples. Six to seven week old female C57BL/6 mice were implanted subcutaneously in the right flank with 1×10³ syngeneic MB49 tumor cells in 100 μl DPBS. After tumors reached 100-150 mm³, typically 7-10 days after tumor inoculation, the mice were treated with the SepGI-162 virus by intra- and peri-tumoral injection of 1×10⁷ pfu of the purified virus in 50 μl DPBS. The treatment groups were as follows: Group 1: treated with formulation buffer (DPBS) only, 3 times per week for 3 weeks (total of 9 treatments); Group 2: treated with the SepGI-Null (control) HSV lacking transgenes, 3 times per week for 3 weeks (total of 9 treatments); Group 3: treated with the SepGI-162 HSV having anti-PD-1/TGFβtrap and IL12 transgenes, 3 times per week for 3 weeks (total of 9 treatments); Group 4: treated with the SepGI-162 HSV 3 times per week for 2 weeks (total of 6 treatments); Group 5: treated with the SepGI-162 HSV 3 times per week for 1 week (total of 3 treatments); Group 6: treated with the SepGI-162 HSV once per week for 3 weeks (total of 3 treatments); and Group 7: treated with the SepGI-162 HSV 3 once per week for 1 week (single treatment). Control Groups 1 and 2 had seven mice each; SepGI-162 treatment Groups 3-7 each had eight mice.

Tumor volumes were measured twice a week using calipers, and body weight was measured once per week. Mice having tumors that reached a size of 2,000 mm³ or that had lost at least 15% of body weight were euthanized. None of the formulation buffer control mice (Group 1) and only one of the SepGI-Null control mice (Group 2) survived to the end of the study. Of the SepGI-162-dosed groups, only one mouse of each of Groups 3 and 5 had to be euthanized and all of the mice of Group 4 survived to the end of the study. These groups received 6 or 9 doses of SepG1-162. Groups 5 and 6, receiving 3 doses of SepG1-162, lost one and two mice, respectively, by the end of the study, and Group 7, receiving only one viral dose was reduced to a single mouse by the end of the study.

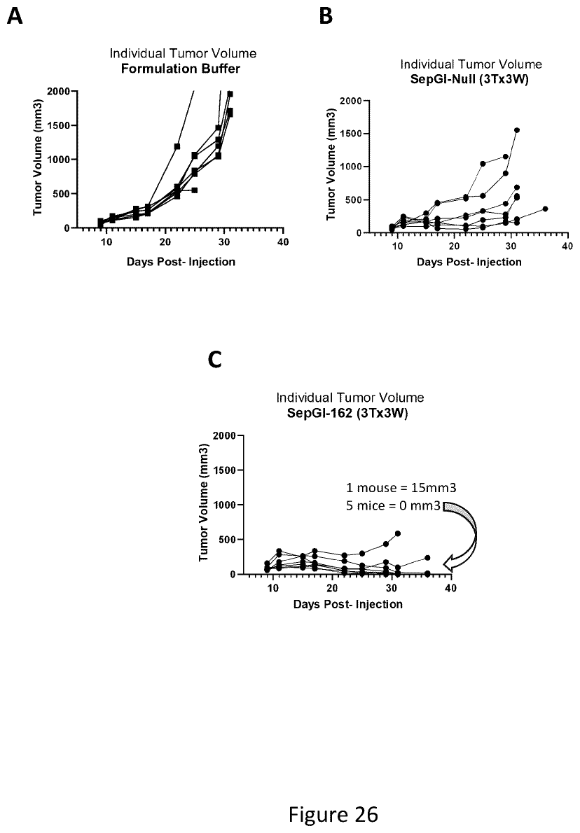

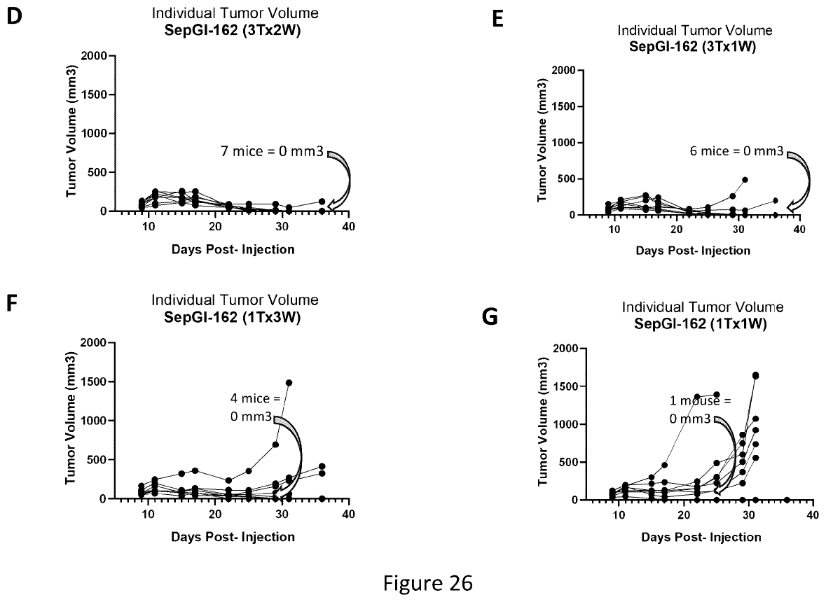

FIGS. 26A-G provide the tumor volumes of the individual mice in the formulation buffer control group, the SepGI-Null treatment group, and the SepGI-162 treatment groups over the course of the study. For all mice in the formulation buffer control group (FIG. 26A), tumor volume increased over the course of the study, increasing dramatically approximately three weeks after inoculation. Overall, mice of the SepGI-Null treatment group experienced a reduced rate of tumor volume increase (FIG. 26B) but tumor was not eradicated in any of the mice of this group.

Group 3, in which the mice received 3 dose per week for 3 consecutive weeks showed complete regression of tumor in 5 of the 8 mice, and near-complete regression in a 6^{th} mouse (tumor measuring only 15 mm³) by the end of the treatment course (FIG. 26C), demonstrating the effectiveness of the anti-PD1/TGFβtrap and IL2 expressing SepGI-162 virus. A very high proportion of the mice of Group 4 (seven of the eight mice), which were dosed three times per week for two weeks rather than three weeks, also demonstrated complete eradication of the MB49 tumor (FIG. 26D), while the eighth mouse showed a significant inhibition of tumor growth. Dosing regimens that provided fewer doses also resulted in tumor regression. For example, treating the mice three times over a single week resulted in tumor eradication in six of the eight mice of Group 5 (FIG. 26E). Dosing once per week over three weeks resulted in eradication of tumor in four of the eight mice of Group 6 (FIG. 26F), while a single dose resulted in complete regression in one mouse of Group 7 (FIG. 26G). Taken together, the dosing study demonstrates the overall effectiveness of the recombinant SepGI-162 virus and a clear dose response.

### Example 23. Dosage Study of SepGI-167 (Anti-PD-L1 ScFv-FcTGFβTrap+IL12) the MB49 Bladder Cancer Xenograft Model in Mice

Another in vivo study was performed to investigate the effect of different dosing regimens of dual gene HSV SepGI-167 that included a Combi5 anti-PD-L1 ScFv-Fc4-TGFβtrap gene and a murine IL12 gene, as described in previous examples. Six to seven week old female C57BL/6 mice were implanted subcutaneously in the right flank with 1×10⁵ syngeneic MB49 tumor cells in 100 μl DPBS. After tumors reached 100-150 mm³, typically 7-10 days after tumor inoculation, the mice were treated with the SepGI-167 virus by intra- and peri-tumoral injection of either 1×10⁵, 1×10⁶, or 1×10⁷ pfu of the purified virus in 50 μl DPBS. The treatment groups (6 mice per group) were as follows: Group 1: treated with formulation buffer (DPBS) only, 3 times per week for 2 weeks (total of 6 treatments); Group 2: treated with 1×10⁷ pfu of the SepGI-167 HSV having anti-PD-L1/TGFβtrap and IL12 transgenes, 3 times per week for 2 weeks (total of 6 treatments); Group 3: treated with 1×10⁶ pfu of the SepGI-167 HSV 3 times per week for 2 weeks (total of 6 treatments); Group 4: treated with 1×10⁵ pfu of the SepGI-167 HSV 3 times per week for 2 weeks (total of 6 treatments); Group 5: treated with 1×10⁷ pfu of the SepGI-167 HSV 3 times over 1 week (total of 3 treatments); Group 6: treated with 1×10⁶ pfu of the SepGI-167 HSV 3 times over 1 week (total of 3 treatments); and Group 7: treated with 1×10⁵ pfu of the SepGI-167 HSV 3 times over 1 week (total of 3 treatments).

Tumor volumes were measured twice a week using calipers, and body weight was measured once per week. Mice having tumors that reached a size of 2,000 mm³ or that had lost at least 15% of body weight were euthanized. None of the formulation buffer control mice (Group 1) survived to the end of the study. Of the groups receiving the lowest dosage of the SepGI-167 virus (1×10⁵ pfu per dose), only one (Group 4) or two (Group 7) mice survived at the end of the study. All of the mice of Group 3, receiving six doses of 1×10⁶ pfu each, survived to the end of the study, as did all of the mice of Group 5 that received 3 doses of 1×10⁷ pfu, and only one mouse of Group 2, receiving 1×10⁷ pfu per dose for a total of 6 doses, had to be euthanized. Two of the mice of Group 6, receiving 3 doses of 1×10⁶ pfu of SepG1-167 had to be euthanized during the study.

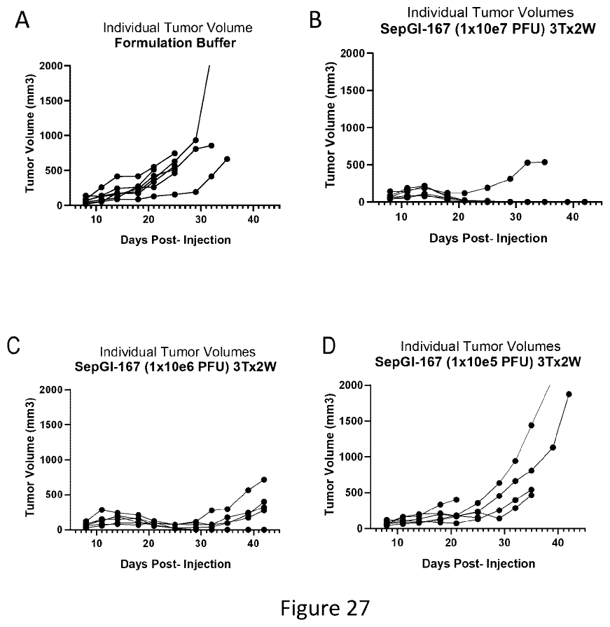

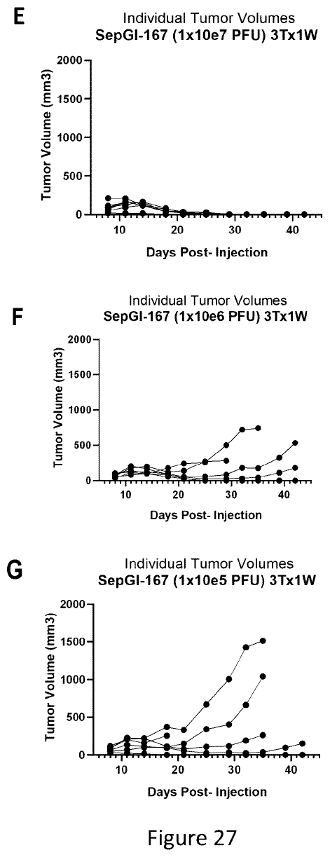

FIGS. 27A-G provide the tumor volumes of the individual mice in the formulation buffer control group and the SepGI-167 treatment groups over the course of the study. For all mice in the formulation buffer control group (FIG. 27A), tumor volume increased over the course of the study, increasing dramatically approximately three weeks after inoculation. None of the mice in control Group 1 survived to the end of the study.

Group 2, in which the mice received 3 doses per week for 3 consecutive weeks at 1×10⁷ pfu per dose, showed complete regression of tumor in 5 of the 6 mice by the end of the treatment course (FIG. 26B), demonstrating the effectiveness of the anti-PDL1/TGFβtrap and IL2 expressing SepGI-167 virus. Two of the mice of Group 3, which received a lower dose (1×10⁶ pfu) of the SepGI-167 virus (also three times per week for two weeks) demonstrated tumor eradication in 2 of the 6 mice (FIG. 26C), while none of the mice of Group 4, receiving the lowest viral dose (1×10⁵ pfu) over the same dosing regimen of 6 treatments, demonstrated complete tumor regression (FIG. 26D).

All of the mice of Group 5, receiving three treatments at the highest dosage level (1×10⁷ pfu) demonstrated complete tumor regression (FIG. 26E). For Group 6, receiving the lower dose of 1×10⁶ pfu for a total of 3 treatments, two mice showed complete tumor regression (FIG. 26F), while three doses at 1×10⁵ pfu per dose resulted in just one mouse of Group 7 experiencing complete regression of tumor (FIG. 26G). Taken together, the dosing study demonstrates the overall effectiveness of the recombinant SepGI-167 virus and a clear dose response.

SEQUENCES

SEQ ID NO: 1
DNA
Artificial
Encodes variant Fc region of IgG4
GAGAGCAAGTACGGCCCCCCCTGCCCCCCCTGCCCCGCCCCCGAGTTCCTGGGCGGCCCCAGCGTGTT
CCTGTTCCCCCCCAAGCCCAAGGACACCCTGATGATCAGCCGCACCCCCGAGGTGACCTGCGTGGTGG
TGGACGTGAGCCAGGAGGACCCCGAGGTGCAGTTCAACTGGTACGTGGACGGCGTGGAGGTGCACAAC
GCCAAGACCAAGCCCCGCGAGGAGCAGTTCAACAGCACCTACCGCGTGGTGAGCGTGCTGACCGTGCT
GCACCAGGACTGGCTGAACGGCAAGGAGTACAAGTGCAAGGTGAGCAACAAGGGCCTGCCCAGCAGCA
TCGAGAAGACCATCAGCAAGGCCAAGGGCCAGCCCCGCGAGCCCCAGGTGTACACCCTGCCCCCCAGC
CAGGAGGAGATGACCAAGAACCAGGTGAGCCTGACCTGCCTGGTGAAGGGCTTCTACCCCAGCGACAT
CGCCGTGGAGTGGGAGAGCAACGGCCAGCCCGAGAACAACTACAAGACCACCCCCCCCGTGCTGGACA
GCGACGGCAGCTTCTTCCTGTACAGCCGCCTGACCGTGGACAAGAGCCGCTGGCAGGAGGGCAACGTG
TTCAGCTGCAGCGTGATGCACGAGGCCCTGCACAACCACTACACCCAGAAGAGCCTGAGCCTGAGCCT
GGGCAAG
SEQ ID NO: 2
protein
Artificial
Variant Fc region of IgG4
ESKYGPPCPPCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNV
FSCSVMHEALHNHYTQKSLSLSLGK
SEQ ID NO: 3
protein
Homo sapiens
Fc region of IgG4
ESKYGPPCPSCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNV
FSCSVMHEALHNHYTQKSLSLSLGK
SEQ ID NO: 4
DNA
Homo sapiens
Encodes Fc region of IgG1
GAACCAAAGTCCTcTGATAAAACACATACTTGCCCACCTTGTCCTGCACCAGAGCTGCTGGGAGGTCC
AAGCGTGTTCCTGTTTCCTCCCAAGCCAAAAGATACTCTTATGATTAGCCGAACACCAGAGGTGACGT
GCGTGGTGGTCGACGTATCACACGAGGACCCTGAAGTGAAGTTTAATTGGTACGTAGACGGGGTTGAG
GTGCATAACGCGAAGACCAAACCTAGAGAGGAGCAGTATAATTCAACCTACCGGGTCGTTTCTGTCCT
CACAGTCCTCCACCAAGATTGGTTGAACGGAAAAGAATATAAGTGTAAAGTGTCTAACAAGGCTCTTC
CCGCGCCTATAGAGAAAACGATCAGCAAAGCGAAGGGGCAACCAAGGGAACCGCAAGTCTACACTCTT
CCACCGTCACGGGATGAGCTGACTAAGAATCAAGTGTCACTTACTTGCCTTGTAAAAGGATTCTACCC
ATCAGACATCGCGGTCGAGTGGGAATCTAACGGTCAACCGGAGAACAATTATAAAACCACTCCTCCCG
TTCTTGACTCCGACGGTTCATTTTTCCTCTATTCCAAACTGACGGTTGATAAGTCTCGATGGCAACAA
GGCAATGTGTTTAGTTGTTCTGTCATGCACGAAGCACTGCACAACCATTATACACAAAAGTCACTTTC
TCTCAGTCCCGGAAAG
SEQ ID NO: 5
protein
Artificial
Variant Fc region of IgG1
Includes C to S mutation at amino acid 5
EPKSSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 6
DNA
Homo sapiens
Encodes ectodomain of TGFβRII
ATTCCTCCTCACGTCCAAAAGTCAGTAAATAATGATATGATAGTCACTGATAATAATGGTGCTGTAAA
GTTCCCACAGCTTTGCAAGTTTTGCGATGTCAGGTTTTCTACCTGCGATAATCAGAAGAGCTGCATGA
GCAATTGCTCTATTACATCCATCTGTGAAAAGCCGCAGGAGGTATGTGTAGCAGTATGGAGAAAGAAC
GATGAAAACATTACACTCGAAACCGTTTGCCACGACCCAAAACTTCCATATCACGATTTTATACTCGA
AGATGCGGCAAGTCCCAAATGCATAATGAAAGAGAAGAAAAAACCAGGCGAAACGTTTTTTATGTGTA
GTTGCAGTAGCGATGAGTGCAACGATAATATAATCTTCTCAGAAGAGTATAACACTTCTAACCCAGAC
SEQ ID NO: 7
protein
Homo sapiens
ectodomain of TGFβRII
IPPHVQKSVNNDMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSEEYNTSNPD
SEQ ID NO: 8
Protein
Artificial
Heavy chain variable region of anti-PD-1 antibody BB9
QVQLVQSGAEVKKPGASVKVSCKASGFRLTTNGISWVRQAPGQGLEWMGWISAGGGPTNYAQKLQGRV
TMTTDTSTSTAYMELRSLRSDDTAVYYCAKGLYGTKDAWGQGTLVTVSS
SEQ ID NO: 9
Protein
Artificial
Light chain variable region of anti-PD-1 antibody BB9
QSVLTQPPSVSEVPGQRVTISCSGGGSNIGSNAVNWYQHFPGKAPKLLIYYNDLLPSGVSDRFSASKS
GTSASLAISGLRSEDEADYYCAAWDDNLSAYVFATGTKVTVL
SEQ ID NO: 10
DNA
Artificial
Encodes BB9 anti-PD-1 monoclonal antibody ScFv (single chain variable region fragment)
ATGGAGTGGAGCTGGGTGTTCCTGTTCTTCCTGAGCGTGACCACCGGCGTGCACAGCCAGGTGCAGCT
GGTGCAGAGCGGCGCCGAGGTGAAGAAGCCCGGCGCCAGCGTGAAGGTGAGCTGCAAGGCCAGCGGCT
TCCGCCTGACCACCAACGGCATCAGCTGGGTGCGCCAGGCCCCCGGCCAGGGCCTGGAGTGGATGGGC
TGGATCAGCGCCGGCGGCGGCCCCACCAACTACGCCCAGAAGCTGCAGGGCCGCGTGACCATGACCAC
CGACACCAGCACCAGCACCGCCTACATGGAGCTGCGCAGCCTGCGCAGCGACGACACCGCCGTGTACT
ACTGCGCCAAGGGCCTGTACGGCACCAAGGACGCCTGGGGCCAGGGCACCCTGGTGACCGTGAGCAGC
GGCGGCGGCGGCAGCGGCGGCGGCGGCAGCGGCGGCGGCGGCAGCCAGAGCGTGCTGACCCAGCCCCC
CAGCGTGAGCGAGGTGCCCGGCCAGCGCGTGACCATCAGCTGCAGCGGCGGCGGCAGCAACATCGGCA
GCAACGCCGTGAACTGGTACCAGCACTTCCCCGGCAAGGCCCCCAAGCTGCTGATCTACTACAACGAC
CTGCTGCCCAGCGGCGTGAGCGACCGCTTCAGCGCCAGCAAGAGCGGCACCAGCGCCAGCCTGGCCAT
CAGCGGCCTGCGCAGCGAGGACGAGGCCGACTACTACTGCGCCGCCTGGGACGACAACCTGAGCGCCT
ACGTGTTCGCCACCGGCACCAAGGTGACCGTGCTG
SEQ ID NO: 11
Protein
Homo sapiens
BB9 anti-PD-1 monoclonal antibody ScFv (single chain variable region fragment)
MEWSWVFLFFLSVTTGVHSQVQLVQSGAEVKKPGASVKVSCKASGFRLTTNGISWVRQAPGQGLEWMG
WISAGGGPTNYAQKLQGRVTMTTDTSTSTAYMELRSLRSDDTAVYYCAKGLYGTKDAWGQGTLVTVSS
GGGGSGGGGSGGGGSQSVLTQPPSVSEVPGQRVTISCSGGGSNIGSNAVNWYQHFPGKAPKLLIYYND
LLPSGVSDRFSASKSGTSASLAISGLRSEDEADYYCAAWDDNLSAYVFATGTKVTVL
SEQ ID NO: 12
Protein
Artificial
Heavy chain variable region of anti-PD-1 antibody RG1H10
QVQLVQSGSELKKPGASVKISCKASGYIFSDNGVNWVRQAPGQGLEWMGWINTKIGNPTYAQGFTGRF
VFSLDTSISTTYLQISSLQAGDTAVYYCAREHDYYYGMDVWGQGTTVTVSS
SEQ ID NO: 13
Protein
Artificial
Light chain variable region of anti-PD-1 antibody RG1H10
QSALTQPPSASGSPGQSVTISCTGTSSDVGGYNYVSWYQHHPGKAPKLMIYEVSKRPSGVPDRFSGSK
SAITASLTISGLLTEDEADYYCSAWDDSLNADVFGGGTKVTVL
SEQ ID NO: 14
DNA
Artificial
Encodes RG1H10 anti-PD-1 monoclonal antibody ScFv (single chain variable region
fragment)
ATGGAGTGGTCTTGGGTATTCCTCTTTTTTCTTAGCGTCACCACAGGTGTTCACTCCCAAGTTCAACT
TGTGCAAAGCGGCTCCGAGTTGAAGAAACCTGGGGCAAGCGTGAAGATCTCATGCAAAGCTTCCGGCT
ATATATTTTCAGACAATGGGGTTAACTGGGTACGCCAGGCCCCGGGTCAGGGACTGGAGTGGATGGGT
TGGATCAATACAAAGATTGGTAATCCTACATACGCACAGGGATTTACGGGGCGATTCGTATTCTCACT
CGATACCTCTATAAGCACAACCTACCTTCAGATAAGCTCCTTGCAAGCAGGTGATACCGCTGTGTACT
ACTGCGCACGGGAACACGACTATTATTACGGTATGGATGTGTGGGGTCAAGGGACCACAGTGACTGTT
AGCAGCGGCGGCGGAGGATCCGGAGGCGGAGGAAGTGGTGGCGGGGGTTCACAAAGCGCTCTCACACA
ACCACCAAGTGCCAGTGGGTCCCCAGGACAAAGTGTTACAATCTCCTGCACTGGGACCTCATCTGATG
TGGGGGGGTACAATTATGTAAGCTGGTACCAACATCATCCAGGAAAGGCTCCAAAATTGATGATCTAT
GAAGTGTCTAAGCGGCCTTCAGGAGTTCCGGACAGGTTCTCTGGGTCAAAAAGTGCCATCACGGCGTC
ACTTACGATTICTGGCCTCCTTACCGAAGATGAAGCCGACTACTACTGCTCAGCATGGGACGATTCCC
TGAACGCGGATGTATTCGGGGGTGGCACCAAAGTTACGGTCCTG
SEQ ID NO: 15
Protein
Artificial
RG1H10 anti-PD-1 monoclonal antibody ScFv (single chain variable region fragment)
MEWSWVFLFFLSVTTGVHSQVQLVQSGSELKKPGASVKISCKASGYIFSDNGVNWVRQAPGQGLEWMG
WINTKIGNPTYAQGFTGRFVFSLDTSISTTYLQISSLQAGDTAVYYCAREHDYYYGMDVWGQGTTVTV
SSGGGGSGGGGSGGGGSQSALTQPPSASGSPGQSVTISCTGTSSDVGGYNYVSWYQHHPGKAPKLMIY
EVSKRPSGVPDRFSGSKSAITASLTISGLLTEDEADYYCSAWDDSLNADVFGGGTKVTVL
SEQ ID NO: 16
Protein
Artificial
Heavy chain variable region of anti-PD-1 antibody pembrolizumab
QVQLVQSGVEVKKPGASVKVSCKASGYTFTNYYMYWVRQAPGQGLEWMGGINPSNGGTNFNEKFKNRV
TLTTDSSTTTAYMELKSLQFDDTAVYYCARRDYRFDMGFDYWGQGTTVTVSS
SEQ ID NO: 17
Protein
Artificial
Light chain variable region of anti-PD-1 antibody pembrolizumab
EIVLTQSPATLSLSPGERATLSCRASKGVSTSGYSYLHWYQQKPGQAPRLLIYLASYLESGVPARFSG
SGSGTDFTLTISSLEPEDFAVYYCQHSRDLPLTFGGGTKVEIKTSENLYFQ
SEQ ID NO: 18
DNA
Artificial
Encodes pembrolizumab anti-PD-1 monoclonal antibody ScFv (single chain variable region
fragment)
ATGGAGTGGAGTTGGGTCTTTCTCTTCTTCCTGTCTGTAACGACTGGCGTGCATTCACAGGTGCAACT
TGTACAGAGCGGCGTTGAAGTGAAAAAACCCGGCGCAAGTGTGAAAGTCAGCTGCAAAGCGTCAGGCT
ACACGTTCACGAATTATTACATGTATTGGGTTAGGCAGGCACCTGGACAGGGGCTGGAGTGGATGGGT
GGCATAAACCCTTCTAATGGCGGAACCAACTTTAACGAGAAGTTTAAGAACCGAGTAACACTCACGAC
TGATAGCAGTACGACCACGGCGTACATGGAACTTAAAAGCCTCCAATTTGACGATACAGCTGTGTACT
ATTGCGCCAGACGCGATTACCGGTTCGACATGGGCTTCGACTATTGGGGTCAGGGAACGACGGTCACA
GTCAGTTCTGGGGGAGGAGGTAGTGGAGGGGGAGGGAGTGGGGGCGGAGGTAGTGAGATAGTTTTGAC
GCAGTCCCCGGCAACTCTGTCCCTGTCACCTGGTGAAAGGGCCACCCTGAGCTGCCGGGCGTCAAAAG
GGGTATCCACGAGCGGATATTCCTATTTGCATTGGTACCAGCAAAAACCCGGTCAGGCTCCGAGGCTT
TTGATTTACTTGGCGTCCTATCTGGAAAGCGGAGTTCCCGCTCGCTTTTCAGGCTCCGGTAGCGGTAC
AGATTTTACTCTCACGATTTCTTCCCTGGAGCCGGAAGACTTTGCGGTATATTATTGTCAGCATAGTC
GCGACCTCCCTCTTACATTCGGAGGAGGTACGAAGGTCGAAATTAAA
SEQ ID NO: 19
Protein
Artificial
pembrolizumab anti-PD-1 monoclonal antibody ScFv (single chain variable region fragment)
MEWSWVFLFFLSVTTGVHSQVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCARRDYRFDMGFDYWGQGTTVT
VSSGGGGSGGGGSGGGGSEIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPLTFGGGTKVEIK
SEQ ID NO: 20
Protein
Artificial
Heavy chain variable region of anti-PD-L1 antibody Combi5
QVQLVQSGAEVKKPGASVKVSCKTSGNTFTNYALHWVRQAPGQGLEWMGGMKPSGGSTSIAQKFQGRV
TMTRDKSTSTVYMELSSLTSEDTAVYYCARDLFPHIFGNYYGMDIWGQGTTVTVSS
SEQ ID NO: 21
Protein
Artificial
Light chain variable region of anti-PD-L1 antibody Combi5
DIVMTQSPPSLSASVGDRVTITCRASQSISSYLNWYQQKPGKAPKLLIYATSSLQYGVPSRFSGSGSG
TDFTLTISSLQPEDFATYYCQGSYSTPYTFGQGTKVEIK
SEQ ID NO: 22
DNA
Artificial
Encodes Combi5 anti-PD-L1 monoclonal antibody ScFv (single chain variable region
fragment)
ATGGAGTGGAGCTGGGTGTTCCTGTTCTTCCTGAGCGTGACCACCGGCGTGCACAGCCAGGTGCAGCT
GGTGCAGAGCGGCGCCGAGGTGAAGAAGCCCGGCGCCAGCGTGAAGGTGAGCTGCAAGACCAGCGGCA
ACACCTTCACCAACTACGCCCTGCACTGGGTGCGCCAGGCCCCCGGCCAGGGCCTGGAGTGGATGGGC
GGCATGAAGCCCAGCGGCGGCAGCACCAGCATCGCCCAGAAGTTCCAGGGCCGCGTGACCATGACCCG
CGACAAGAGCACCAGCACCGTGTACATGGAGCTGAGCAGCCTGACCAGCGAGGACACCGCCGTGTACT
ACTGCGCCCGCGACCTGTTCCCCCACATCTTCGGCAACTACTACGGCATGGACATCTGGGGCCAGGGC
ACCACCGTGACCGTGAGCAGCGGCGGCGGCGGCAGCGGCGGCGGCGGCAGCGGCGGCGGCGGCAGCGA
CATCGTGATGACCCAGAGCCCCCCCAGCCTGAGCGCCAGCGTGGGCGACCGCGTGACCATCACCTGCC
GCGCCAGCCAGAGCATCAGCAGCTACCTGAACTGGTACCAGCAGAAGCCCGGCAAGGCCCCCAAGCTG
CTGATCTACGCCACCAGCAGCCTGCAGTACGGCGTGCCCAGCCGCTTCAGCGGCAGCGGCAGCGGCAC
CGACTTCACCCTGACCATCAGCAGCCTGCAGCCCGAGGACTTCGCCACCTACTACTGCCAGGGCAGCT
ACAGCACCCCCTACACCTTCGGCCAGGGCACCAAGGTGGAGATCAAG
SEQ ID NO: 23
Protein
Artificial
Combi5 anti-PD-L1 monoclonal antibody ScFv (single chain variable region fragment)
MEWSWVFLFFLSVTTGVHSQVQLVQSGAEVKKPGASVKVSCKTSGNTFTNYALHWVRQAPGQGLEWMG
GMKPSGGSTSIAQKFQGRVTMTRDKSTSTVYMELSSLTSEDTAVYYCARDLFPHIFGNYYGMDIWGQG
TTVTVSSGGGGSGGGGSGGGGSDIVMTQSPPSLSASVGDRVTITCRASQSISSYLNWYQQKPGKAPKL
LIYATSSLQYGVPSRFSGSGSGTDFTLTISSLQPEDFATYYCQGSYSTPYTFGQGTKVEIK
SEQ ID NO: 24
Protein
Artificial
Heavy chain variable domain of anti-PD-L1 antibody H6B1LEM
QMQLVQSGAEVKKPGSSVKVSCKASGGTFSSYAYSWVRQAPGQGLEWMGGIIPSFGTANYAQKFQGRV
TITADESTSTAYMELSSLRSEDTAVYYCARGPIVATITPLDYWGQGTLVTVSS
SEQ ID NO: 25
Protein
Artificial
Light chain variable domain of anti-PD-L1 antibody H6B1LEM
SYVLTQPPSVSVAPGKTATIACGGENIGRKTVHWYQQKPGQAPVLVIYYDSDRPSGIPERFSGSNSGN
TATLTISRVEAGDEADYYCLVWDSSSDHRIFGGGTKLTVL
SEQ ID NO: 26
DNA
Artificial
Encodes H6B1LEM anti-PD-L1 monoclonal antibody ScFv (single chain variable region
fragment)
ATGGAGTGGAGCTGGGTGTTCCTGTTCTTCCTGAGCGTGACCACCGGCGTGCACAGCCAGATGCAGCT
GGTGCAGAGCGGCGCCGAGGTGAAGAAGCCCGGCAGCAGCGTGAAGGTGAGCTGCAAGGCCAGCGGCG
GCACCTTCAGCAGCTACGCCTACAGCTGGGTGCGCCAGGCCCCCGGCCAGGGCCTGGAGTGGATGGGC
GGCATCATCCCCAGCTTCGGCACCGCCAACTACGCCCAGAAGTTCCAGGGCCGCGTGACCATCACCGC
CGACGAGAGCACCAGCACCGCCTACATGGAGCTGAGCAGCCTGCGCAGCGAGGACACCGCCGTGTACT
ACTGCGCCCGCGGCCCCATCGTGGCCACCATCACCCCCCTGGACTACTGGGGCCAGGGCACCCTGGTG
ACCGTGAGCAGCGGCGGCGGCGGCAGCGGCGGCGGCGGCAGCGGCGGCGGCGGCAGCAGCTACGTGCT
GACCCAGCCCCCCAGCGTGAGCGTGGCCCCCGGCAAGACCGCCACCATCGCCTGCGGCGGCGAGAACA
TCGGCCGCAAGACCGTGCACTGGTACCAGCAGAAGCCCGGCCAGGCCCCCGTGCTGGTGATCTACTAC
GACAGCGACCGCCCCAGCGGCATCCCCGAGCGCTTCAGCGGCAGCAACAGCGGCAACACCGCCACCCT
GACCATCAGCCGCGTGGAGGCCGGCGACGAGGCCGACTACTACTGCCTGGTGTGGGACAGCAGCAGCG
ACCACCGCATCTTCGGCGGCGGCACCAAGCTGACCGTGCTG
SEQ ID NO: 27
Protein
Artificial
H6B1LEM anti-PD-L1 monoclonal antibody ScFv (single chain variable region fragment)
MEWSWVFLFFLSVTTGVHSQMQLVQSGAEVKKPGSSVKVSCKASGGTFSSYAYSWVRQAPGQGLEWMG
GIIPSFGTANYAQKFQGRVTITADESTSTAYMELSSLRSEDTAVYYCARGPIVATITPLDYWGQGTLV
TVSSGGGGSGGGGSGGGGSSYVLTQPPSVSVAPGKTATIACGGENIGRKTVHWYQQKPGQAPVLVIYY
DSDRPSGIPERFSGSNSGNTATLTISRVEAGDEADYYCLVWDSSSDHRIFGGGTKLTVL
SEQ ID NO: 28
Protein
Artificial
Heavy chain variable domain of anti-PD-L1 antibody avelumab
SEVQLLESGGGLVQPGGSLRLSCAASGFTFSSYIMMWVRQAPGKGLEWVSSIYPSGGITFYADTVKGRFTISRDNS
KNTLYLQMNSLRAEDTAVYYCARIKLGTVTTVDYWGQGTLVTVSS
SEQ ID NO: 29
Protein
Artificial
Light chain variable domain of anti-PD-L1 antibody avelumab
QSALTQPASVSGSPGQSITISCTGTSSDVGGYNYVSWYQQHPGKAPKLMIYDVSNRPSGVSNRFSGSKSGNTASLT
ISGLQAEDEADYYCSSYTSSSTRVFGTGTKVTVL
SEQ ID NO: 30
DNA
Artificial
Encodes avelumab anti-PD-L1 ScFv (single chain variable region fragment)
ATGGAATGGTCCTGGGTCTTCCTTTTCTTCCTCTCTGTTACCACTGGCGTTCACAGCCAATCAGCACT
TACGCAACCTGCTAGCGTTAGTGGTAGTCCAGGTCAGAGCATCACAATTTCATGTACTGGGACCTCCA
GCGATGTAGGAGGATACAACTACGTGTCATGGTATCAGCAGCACCCCGGCAAGGCCCCTAAGCTTATG
ATCTACGACGTCTCAAACAGGCCCAGTGGTGTTAGTAACAGGTTTAGCGGATCAAAATCTGGAAATAC
AGCGAGTCTCACTATTTCCGGGTTGCAAGCTGAGGATGAAGCTGACTATTATTGTTCTTCATACACAT
CTTCATCAACAAGAGTATTTGGGACAGGAACAAAAGTGACAGTCTTGGGTGGTGGAGGCAGTGGGGGA
GGAGGTAGTGGCGGCGGTGGGTCAGAAGTCCAACTCTTGGAAAGCGGCGGGGGCCTCGTACAACCTGG
TGGCAGTCTGAGACTGTCATGCGCGGCAAGCGGATTCACTTTCTCATCTTATATAATGATGTGGGTTA
GACAAGCGCCAGGGAAAGGCTTGGAATGGGTAAGTTCAATCTACCCGTCTGGCGGGATTACGTTCTAC
GCTGATACGGTGAAAGGGAGGTTCACGATTTCTCGAGACAACTCCAAGAATACGCTGTATCTTCAGAT
GAACTCTCTCCGAGCCGAAGATACCGCAGTATACTACTGCGCACGCATTAAACTGGGCACCGTTACCA
CAGTTGATTACTGGGGCCAGGGTACGCTCGTGACGGTCTCTAGC
SEQ ID NO: 31
Protein
Artificial
avelumab anti-PD-L1 ScFv (single chain variable region fragment)
MEWSWVFLFFLSVTTGVHSQSALTQPASVSGSPGQSITISCTGTSSDVGGYNYVSWYQQHPGKAPKLM
IYDVSNRPSGVSNRFSGSKSGNTASLTISGLQAEDEADYYCSSYTSSSTRVFGTGTKVTVLGGGGSGG
GGSGGGGSEVQLLESGGGLVQPGGSLRLSCAASGFTFSSYIMMWVRQAPGKGLEWVSSIYPSGGITFY
ADTVKGRFTISRDNSKNTLYLQMNSLRAEDTAVYYCARIKLGTVTTVDYWGQGTLVTVSS
SEQ ID NO: 32
DNA
Artificial
EF1a/HTLV hybrid promoter
AAGGATCTGCGATCGCTCCGGTGCCCGTCAGTGGGCAGAGCGCACATCGCCCACAGTCCCCGAGAAGT
TGGGGGGAGGGGTCGGCAATTGAACGGGTGCCTAGAGAAGGTGGCGCGGGGTAAACTGGGAAAGTGAT
GTCGTGTACTGGCTCCGCCTTTTTCCCGAGGGTGGGGGAGAACCGTATATAAGTGCAGTAGTCGCCGT
GAACGTTCTTTTTCGCAACGGGTTTGCCGCCAGAACACAGCTGAAGCTTCGAGGGGCTCGCATCTCTC
CTTCACGCGCCCGCCGCCCTACCTGAGGCCGCCATCCACGCCGGTTGAGTCGCGTTCTGCCGCCTCCC
GCCTGTGGTGCCTCCTGAACTGCGTCCGCCGTCTAGGTAAGTTTAAAGCTCAGGTCGAGACCGGGCCT
TTGTCCGGCGCTCCCTTGGAGCCTACCTAGACTCAGCCGGCTCTCCACGCTTTGCCTGACCCTGCTTG
CTCAACTCTACGTCTTTGTTTCGTTTTCTGTTCTGCGCCGTTACAGATC
SEQ ID NO: 33
DNA
Cytomegalovirus
CMV promoter
AAGCTTGGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGACGTCA
ATGGGTGGAGTATTTACGGTAAACTGCCCACTTGGCAGTACATCAAGTGTATCATATGCCAAGTACGC
CCCCTATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATG
GGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGAGCTCGTTTAGTGAACCGTCAGAT
CGCCTGGAGACGCCATCCACGCTGTTTTGACCTCCATAGAAGACACCGACTCTACTAGA
SEQ ID NO: 34
Protein
Mus musculus
Signal Peptide, IgG heavy chain
MEWSWVFLFFLSVTTGVHS
SEQ ID NO: 35
Protein
Artificial
Signal Peptide
MEFGLSWVFLVALFRGVQCD
SEQ ID NO: 36
Protein
Artificial
Signal Peptide
METDTLLLWVLLLWVP
SEQ ID NO: 37
DNA
Bos taurus
BGH Poly A addition sequence
CTGTGCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAGACAATAGCAGGCATGCTG
GGGATGCGGTGGGCTCTATGG
SEQ ID NO: 38
DNA
SV40
SV40 PolyA addition sequence
TAGATAACTGATCATAATCAGCCATACCACATTTGTAGAGGTTTTACTTGCTTTAAAAAACCTCCCAC
ACCTCCCCCTGAACCTGAAACATAAAATGAATGCAATTGTTGTTGTTAACTTGTTTATTGCAGCTTAT
AATGGTTACAAATAAAGCAATAGCATCACAAATTTCACAAATAAAGCATTTTTTTCACTGCATTCTAG
TTGTGGTTTGTCCAAACTCATCAATGTATCTTA
SEQ ID NO: 39
DNA
Artificial
BB9 anti-PD-1 ScFv-Fc4-TGFβRII_{ecto} construct
ATGGAGTGGAGCTGGGTGTTCCTGTTCTTCCTGAGCGTGACCACCGGCGTGCACAGCCAGGTGCAGCT
GGTGCAGAGCGGCGCCGAGGTGAAGAAGCCCGGCGCCAGCGTGAAGGTGAGCTGCAAGGCCAGCGGCT
TCCGCCTGACCACCAACGGCATCAGCTGGGTGCGCCAGGCCCCCGGCCAGGGCCTGGAGTGGATGGGC
TGGATCAGCGCCGGCGGCGGCCCCACCAACTACGCCCAGAAGCTGCAGGGCCGCGTGACCATGACCAC
CGACACCAGCACCAGCACCGCCTACATGGAGCTGCGCAGCCTGCGCAGCGACGACACCGCCGTGTACT
ACTGCGCCAAGGGCCTGTACGGCACCAAGGACGCCTGGGGCCAGGGCACCCTGGTGACCGTGAGCAGC
GGCGGCGGCGGCAGCGGCGGCGGCGGCAGCGGCGGCGGCGGCAGCCAGAGCGTGCTGACCCAGCCCCC
CAGCGTGAGCGAGGTGCCCGGCCAGCGCGTGACCATCAGCTGCAGCGGCGGCGGCAGCAACATCGGCA
GCAACGCCGTGAACTGGTACCAGCACTTCCCCGGCAAGGCCCCCAAGCTGCTGATCTACTACAACGAC
CTGCTGCCCAGCGGCGTGAGCGACCGCTTCAGCGCCAGCAAGAGCGGCACCAGCGCCAGCCTGGCCAT
CAGCGGCCTGCGCAGCGAGGACGAGGCCGACTACTACTGCGCCGCCTGGGACGACAACCTGAGCGCCT
ACGTGTTCGCCACCGGCACCAAGGTGACCGTGCTGGAGAGCAAGTACGGCCCCCCCTGCCCCCCCTGC
CCCGCCCCCGAGTTCCTGGGCGGCCCCAGCGTGTTCCTGTTCCCCCCCAAGCCCAAGGACACCCTGAT
GATCAGCCGCACCCCCGAGGTGACCTGCGTGGTGGTGGACGTGAGCCAGGAGGACCCCGAGGTGCAGT
TCAACTGGTACGTGGACGGCGTGGAGGTGCACAACGCCAAGACCAAGCCCCGCGAGGAGCAGTTCAAC
AGCACCTACCGCGTGGTGAGCGTGCTGACCGTGCTGCACCAGGACTGGCTGAACGGCAAGGAGTACAA
GTGCAAGGTGAGCAACAAGGGCCTGCCCAGCAGCATCGAGAAGACCATCAGCAAGGCCAAGGGCCAGC
CCCGCGAGCCCCAGGTGTACACCCTGCCCCCCAGCCAGGAGGAGATGACCAAGAACCAGGTGAGCCTG
ACCTGCCTGGTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACAGCCGCCTGA
CCGTGGACAAGAGCCGCTGGCAGGAGGGCAACGTGTTCAGCTGCAGCGTGATGCACGAGGCCCTGCAC
AACCACTACACCCAGAAGAGCCTGAGCCTGAGCCTGGGCAAGGGAGGTGGCGGGTCAGGCGGAGGTGG
TAGTGGCGGCGGTGGGAGTATTCCTCCTCACGTCCAAAAGTCAGTAAATAATGATATGATAGTCACTG
ATAATAATGGTGCTGTAAAGTTCCCACAGCTTTGCAAGTTTTGCGATGTCAGGTTTTCTACCTGCGAT
AATCAGAAGAGCTGCATGAGCAATTGCTCTATTACATCCATCTGTGAAAAGCCGCAGGAGGTATGTGT
AGCAGTATGGAGAAAGAACGATGAAAACATTACACTCGAAACCGTTTGCCACGACCCAAAACTTCCAT
ATCACGATTTTATACTCGAAGATGCGGCAAGTCCCAAATGCATAATGAAAGAGAAGAAAAAACCAGGC
GAAACGTTTTTTATGTGTAGTTGCAGTAGCGATGAGTGCAACGATAATATAATCTTCTCAGAAGAGTA
TAACACTTCTAACCCAGAC
SEQ ID NO: 40
Protein
Artificial
BB9 anti-PD-1 ScFv-Fc4-TGFβRII_{ecto} fusion protein
MEWSWVFLFFLSVTTGVHSQVQLVQSGAEVKKPGASVKVSCKASGFRLTTNGISWVRQAPGQGLEWMG
WISAGGGPTNYAQKLQGRVTMTTDTSTSTAYMELRSLRSDDTAVYYCAKGLYGTKDAWGQGTLVTVSS
GGGGSGGGGSGGGGSQSVLTQPPSVSEVPGQRVTISCSGGGSNIGSNAVNWYQHFPGKAPKLLIYYND
LLPSGVSDRFSASKSGTSASLAISGLRSEDEADYYCAAWDDNLSAYVFATGTKVTVLESKYGPPCPPC
PAPEFLGGPSVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSVMHEALH
NHYTQKSLSLSLGKGGGGSGGGGSGGGGSIPPHVQKSVNNDMIVTDNNGAVKFPQLCKFCDVRFSTCD
NQKSCMSNCSITSICEKPQEVCVAVWRKNDENITLETVCHDPKLPYHDFILEDAASPKCIMKEKKKPG
ETFFMCSCSSDECNDNIIFSEEYNTSNPD
SEQ ID NO: 41
DNA
Artificial
RG1H10 anti-PD-1 ScFv-Fc4-TGFβRII_{ecto} construct
ATGGAGTGGTCTTGGGTATTCCTCTTTTTTCTTAGCGTCACCACAGGTGTTCACTCCCAAGTTCAACT
TGTGCAAAGCGGCTCCGAGTTGAAGAAACCTGGGGCAAGCGTGAAGATCTCATGCAAAGCTTCCGGCT
ATATATTTTCAGACAATGGGGTTAACTGGGTACGCCAGGCCCCGGGTCAGGGACTGGAGTGGATGGGT
TGGATCAATACAAAGATTGGTAATCCTACATACGCACAGGGATTTACGGGGCGATTCGTATTCTCACT
CGATACCTCTATAAGCACAACCTACCTTCAGATAAGCTCCTTGCAAGCAGGTGATACCGCTGTGTACT
ACTGCGCACGGGAACACGACTATTATTACGGTATGGATGTGTGGGGTCAAGGGACCACAGTGACTGTT
AGCAGCGGCGGCGGAGGATCCGGAGGCGGAGGAAGTGGTGGCGGGGGTTCACAAAGCGCTCTCACACA
ACCACCAAGTGCCAGTGGGTCCCCAGGACAAAGTGTTACAATCTCCTGCACTGGGACCTCATCTGATG
TGGGGGGGTACAATTATGTAAGCTGGTACCAACATCATCCAGGAAAGGCTCCAAAATTGATGATCTAT
GAAGTGTCTAAGCGGCCTTCAGGAGTTCCGGACAGGTTCTCTGGGTCAAAAAGTGCCATCACGGCGTC
ACTTACGATTTCTGGCCTCCTTACCGAAGATGAAGCCGACTACTACTGCTCAGCATGGGACGATTCCC
TGAACGCGGATGTATTCGGGGGTGGCACCAAAGTTACGGTCCTGGAGAGCAAGTACGGCCCCCCCTGC
CCCCCCTGCCCCGCCCCCGAGTTCCTGGGCGGCCCCAGCGTGTTCCTGTTCCCCCCCAAGCCCAAGGA
CACCCTGATGATCAGCCGCACCCCCGAGGTGACCTGCGTGGTGGTGGACGTGAGCCAGGAGGACCCCG
AGGTGCAGTTCAACTGGTACGTGGACGGCGTGGAGGTGCACAACGCCAAGACCAAGCCCCGCGAGGAG
CAGTTCAACAGCACCTACCGCGTGGTGAGCGTGCTGACCGTGCTGCACCAGGACTGGCTGAACGGCAA
GGAGTACAAGTGCAAGGTGAGCAACAAGGGCCTGCCCAGCAGCATCGAGAAGACCATCAGCAAGGCCA
AGGGCCAGCCCCGCGAGCCCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGTGGAGTGGGAGAGCAACGG
CCAGCCCGAGAACAACTACAAGACCACCCCCCCCGTGCTGGACAGCGACGGCAGCTTCTTCCTGTACA
GCCGCCTGACCGTGGACAAGAGCCGCTGGCAGGAGGGCAACGTGTTCAGCTGCAGCGTGATGCACGAG
GCCCTGCACAACCACTACACCCAGAAGAGCCTGAGCCTGAGCCTGGGCAAGGGAGGTGGCGGGTCAGG
CGGAGGTGGTAGTGGCGGCGGTGGGAGTATTCCTCCTCACGTCCAAAAGTCAGTAAATAATGATATGA
TAGTCACTGATAATAATGGTGCTGTAAAGTTCCCACAGCTTTGCAAGTTTTGCGATGTCAGGTTTTCT
ACCTGCGATAATCAGAAGAGCTGCATGAGCAATTGCTCTATTACATCCATCTGTGAAAAGCCGCAGGA
GGTATGTGTAGCAGTATGGAGAAAGAACGATGAAAACATTACACTCGAAACCGTTTGCCACGACCCAA
AACTTCCATATCACGATTTTATACTCGAAGATGCGGCAAGTCCCAAATGCATAATGAAAGAGAAGAAA
AAACCAGGCGAAACGTTTTTTATGTGTAGTTGCAGTAGCGATGAGTGCAACGATAATATAATCTTCTC
AGAAGAGTATAACACTTCTAACCCAGAC
SEQ ID NO: 42
Protein
Artificial
RG1H10 anti-PD-1 ScFv-Fc4-TGFβRII_{ecto} fusion protein
MEWSWVFLFFLSVTTGVHSQVQLVQSGSELKKPGASVKISCKASGYIFSDNGVNWVRQAPGQGLEWMG
WINTKIGNPTYAQGFTGRFVFSLDTSISTTYLQISSLQAGDTAVYYCAREHDYYYGMDVWGQGTTVTV
SSGGGGSGGGGSGGGGSQSALTQPPSASGSPGQSVTISCTGTSSDVGGYNYVSWYQHHPGKAPKLMIY
EVSKRPSGVPDRFSGSKSAITASLTISGLLTEDEADYYCSAWDDSLNADVFGGGTKVTVLESKYGPPC
PPCPAPEFLGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSVMHE
ALHNHYTQKSLSLSLGKGGGGSGGGGSGGGGSIPPHVQKSVNNDMIVTDNNGAVKFPQLCKFCDVRFS
TCDNQKSCMSNCSITSICEKPQEVCVAVWRKNDENITLETVCHDPKLPYHDFILEDAASPKCIMKEKK
KPGETFFMCSCSSDECNDNIIFSEEYNTSNPD
SEQ ID NO: 43
DNA
Artificial
pembrolizumab anti-PD-1 ScFv-Fc4-TGFβRII_{ecto} construct
ATGGAGTGGAGTTGGGTCTTTCTCTTCTTCCTGTCTGTAACGACTGGCGTGCATTCACAGGTGCAACT
TGTACAGAGCGGCGTTGAAGTGAAAAAACCCGGCGCAAGTGTGAAAGTCAGCTGCAAAGCGTCAGGCT
ACACGTTCACGAATTATTACATGTATTGGGTTAGGCAGGCACCTGGACAGGGGCTGGAGTGGATGGGT
GGCATAAACCCTTCTAATGGCGGAACCAACTTTAACGAGAAGTTTAAGAACCGAGTAACACTCACGAC
TGATAGCAGTACGACCACGGCGTACATGGAACTTAAAAGCCTCCAATTTGACGATACAGCTGTGTACT
ATTGCGCCAGACGCGATTACCGGTTCGACATGGGCTTCGACTATTGGGGTCAGGGAACGACGGTCACA
GTCAGTTCTGGGGGAGGAGGTAGTGGAGGGGGAGGGAGTGGGGGCGGAGGTAGTGAGATAGTTTTGAC
GCAGTCCCCGGCAACTCTGTCCCTGTCACCTGGTGAAAGGGCCACCCTGAGCTGCCGGGCGTCAAAAG
GGGTATCCACGAGCGGATATTCCTATTTGCATTGGTACCAGCAAAAACCCGGTCAGGCTCCGAGGCTT
TTGATTTACTTGGCGTCCTATCTGGAAAGCGGAGTTCCCGCTCGCTTTTCAGGCTCCGGTAGCGGTAC
AGATTTTACTCTCACGATTTCTTCCCTGGAGCCGGAAGACTTTGCGGTATATTATTGTCAGCATAGTC
GCGACCTCCCTCTTACATTCGGAGGAGGTACGAAGGTCGAAATTAAAGAGAGCAAGTACGGCCCCCCC
TGCCCCCCCTGCCCCGCCCCCGAGTTCCTGGGCGGCCCCAGCGTGTTCCTGTTCCCCCCCAAGCCCAA
GGACACCCTGATGATCAGCCGCACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAGCCCCGCGAG
GAGCAGTTCAACAGCACCTACCGCGTGGTGAGCGTGCTGACCGTGCTGCACCAGGACTGGCTGAACGG
CAAGGAGTACAAGTGCAAGGTGAGCAACAAGGGCCTGCCCAGCAGCATCGAGAAGACCATCAGCAAGG
CCAAGGGCCAGCCCCGCGAGCCCCAGGTGTACACCCTGCCCCCCAGCCAGGAGGAGATGACCAAGAAC
CAGGTGAGCCTGACCTGCCTGGTGAAGGGCTTCTACCCCAGCGACATCGCCGTGGAGTGGGAGAGCAA
CGGCCAGCCCGAGAACAACTACAAGACCACCCCCCCCGTGCTGGACAGCGACGGCAGCTTCTTCCTGT
ACAGCCGCCTGACCGTGGACAAGAGCCGCTGGCAGGAGGGCAACGTGTTCAGCTGCAGCGTGATGCAC
GAGGCCCTGCACAACCACTACACCCAGAAGAGCCTGAGCCTGAGCCTGGGCAAGGGAGGTGGCGGGTC
AGGCGGAGGTGGTAGTGGCGGCGGTGGGAGTATTCCTCCTCACGTCCAAAAGTCAGTAAATAATGATA
TGATAGTCACTGATAATAATGGTGCTGTAAAGTTCCCACAGCTTTGCAAGTTTTGCGATGTCAGGTTT
TCTACCTGCGATAATCAGAAGAGCTGCATGAGCAATTGCTCTATTACATCCATCTGTGAAAAGCCGCA
GGAGGTATGTGTAGCAGTATGGAGAAAGAACGATGAAAACATTACACTCGAAACCGTTTGCCACGACC
CAAAACTTCCATATCACGATTTTATACTCGAAGATGCGGCAAGTCCCAAATGCATAATGAAAGAGAAG
AAAAAACCAGGCGAAACGTTTTTTATGTGTAGTTGCAGTAGCGATGAGTGCAACGATAATATAATCTT
CTCAGAAGAGTATAACACTTCTAACCCAGAC
SEQ ID NO: 44
Protein
Artificial
pembrolizumab anti-PD-1 ScFv-Fc4-TGFβRII_{ecto} fusion protein
MEWSWVFLFFLSVTTGVHSQVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCARRDYRFDMGFDYWGQGTTVT
VSSGGGGSGGGGSGGGGSEIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPLTFGGGTKVEIKESKYGPP
CPPCPAPEFLGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFSCSVMH
EALHNHYTQKSLSLSLGKGGGGSGGGGSGGGGSIPPHVQKSVNNDMIVTDNNGAVKFPQLCKFCDVRF
STCDNQKSCMSNCSITSICEKPQEVCVAVWRKNDENITLETVCHDPKLPYHDFILEDAASPKCIMKEK
KKPGETFFMCSCSSDECNDNIIFSEEYNTSNPD
SEQ ID NO: 45
DNA
Artificial
Combi5 anti-PD-L1 ScFv-Fc4-TGFβRII_{ecto} construct
ATGGAGTGGAGCTGGGTGTTCCTGTTCTTCCTGAGCGTGACCACCGGCGTGCACAGCCAGGTGCAGCT
GGTGCAGAGCGGCGCCGAGGTGAAGAAGCCCGGCGCCAGCGTGAAGGTGAGCTGCAAGACCAGCGGCA
ACACCTTCACCAACTACGCCCTGCACTGGGTGCGCCAGGCCCCCGGCCAGGGCCTGGAGTGGATGGGC
GGCATGAAGCCCAGCGGCGGCAGCACCAGCATCGCCCAGAAGTTCCAGGGCCGCGTGACCATGACCCG
CGACAAGAGCACCAGCACCGTGTACATGGAGCTGAGCAGCCTGACCAGCGAGGACACCGCCGTGTACT
ACTGCGCCCGCGACCTGTTCCCCCACATCTTCGGCAACTACTACGGCATGGACATCTGGGGCCAGGGC
ACCACCGTGACCGTGAGCAGCGGCGGCGGCGGCAGCGGCGGCGGCGGCAGCGGCGGCGGCGGCAGCGA
CATCGTGATGACCCAGAGCCCCCCCAGCCTGAGCGCCAGCGTGGGCGACCGCGTGACCATCACCTGCC
GCGCCAGCCAGAGCATCAGCAGCTACCTGAACTGGTACCAGCAGAAGCCCGGCAAGGCCCCCAAGCTG
CTGATCTACGCCACCAGCAGCCTGCAGTACGGCGTGCCCAGCCGCTTCAGCGGCAGCGGCAGCGGCAC
CGACTTCACCCTGACCATCAGCAGCCTGCAGCCCGAGGACTTCGCCACCTACTACTGCCAGGGCAGCT
ACAGCACCCCCTACACCTTCGGCCAGGGCACCAAGGTGGAGATCAAGGAGAGCAAGTACGGCCCCCCC
TGCCCCCCCTGCCCCGCCCCCGAGTTCCTGGGCGGCCCCAGCGTGTTCCTGTTCCCCCCCAAGCCCAA
GGACACCCTGATGATCAGCCGCACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAGCCCCGCGAG
GAGCAGTTCAACAGCACCTACCGCGTGGTGAGCGTGCTGACCGTGCTGCACCAGGACTGGCTGAACGG
CAAGGAGTACAAGTGCAAGGTGAGCAACAAGGGCCTGCCCAGCAGCATCGAGAAGACCATCAGCAAGG
CCAAGGGCCAGCCCCGCGAGCCCCAGGTGTACACCCTGCCCCCCAGCCAGGAGGAGATGACCAAGAAC
CAGGTGAGCCTGACCTGCCTGGTGAAGGGCTTCTACCCCAGCGACATCGCCGTGGAGTGGGAGAGCAA
CGGCCAGCCCGAGAACAACTACAAGACCACCCCCCCCGTGCTGGACAGCGACGGCAGCTTCTTCCTGT
ACAGCCGCCTGACCGTGGACAAGAGCCGCTGGCAGGAGGGCAACGTGTTCAGCTGCAGCGTGATGCAC
GAGGCCCTGCACAACCACTACACCCAGAAGAGCCTGAGCCTGAGCCTGGGCAAGGGAGGTGGCGGGTC
AGGCGGAGGTGGTAGTGGCGGCGGTGGGAGTATTCCTCCTCACGTCCAAAAGTCAGTAAATAATGATA
TGATAGTCACTGATAATAATGGTGCTGTAAAGTTCCCACAGCTTTGCAAGTTTTGCGATGTCAGGTTT
TCTACCTGCGATAATCAGAAGAGCTGCATGAGCAATTGCTCTATTACATCCATCTGTGAAAAGCCGCA
GGAGGTATGTGTAGCAGTATGGAGAAAGAACGATGAAAACATTACACTCGAAACCGTTTGCCACGACC
CAAAACTTCCATATCACGATTTTATACTCGAAGATGCGGCAAGTCCCAAATGCATAATGAAAGAGAAG
AAAAAACCAGGCGAAACGTTTTTTATGTGTAGTTGCAGTAGCGATGAGTGCAACGATAATATAATCTT
CTCAGAAGAGTATAACACTTCTAACCCAGAC
SEQ ID NO: 46
Protein
Artificial
Combi5 anti-PD-L1 ScFv-Fc4-TGFβRII_{ecto} fusion protein
MEWSWVFLFFLSVTTGVHSQVQLVQSGAEVKKPGASVKVSCKTSGNTFTNYALHWVRQAPGQGLEWMG
GMKPSGGSTSIAQKFQGRVTMTRDKSTSTVYMELSSLTSEDTAVYYCARDLFPHIFGNYYGMDIWGQG
TTVTVSSGGGGSGGGGSGGGGSDIVMTQSPPSLSASVGDRVTITCRASQSISSYLNWYQQKPGKAPKL
LIYATSSLQYGVPSRFSGSGSGTDFTLTISSLQPEDFATYYCQGSYSTPYTFGQGTKVEIKESKYGPP
CPPCPAPEFLGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFSCSVMH
EALHNHYTQKSLSLSLGKGGGGSGGGGSGGGGSIPPHVQKSVNNDMIVTDNNGAVKFPQLCKFCDVRF
STCDNQKSCMSNCSITSICEKPQEVCVAVWRKNDENITLETVCHDPKLPYHDFILEDAASPKCIMKEK
KKPGETFFMCSCSSDECNDNIIFSEEYNTSNPD
SEQ ID NO: 47
DNA
Artificial
H6B1LEM anti-PD-L1 ScFv-Fc4-TGFβRII_{ecto} construct
ATGGAGTGGAGCTGGGTGTTCCTGTTCTTCCTGAGCGTGACCACCGGCGTGCACAGCCAGATGCAGCT
GGTGCAGAGCGGCGCCGAGGTGAAGAAGCCCGGCAGCAGCGTGAAGGTGAGCTGCAAGGCCAGCGGCG
GCACCTTCAGCAGCTACGCCTACAGCTGGGTGCGCCAGGCCCCCGGCCAGGGCCTGGAGTGGATGGGC
GGCATCATCCCCAGCTTCGGCACCGCCAACTACGCCCAGAAGTTCCAGGGCCGCGTGACCATCACCGC
CGACGAGAGCACCAGCACCGCCTACATGGAGCTGAGCAGCCTGCGCAGCGAGGACACCGCCGTGTACT
ACTGCGCCCGCGGCCCCATCGTGGCCACCATCACCCCCCTGGACTACTGGGGCCAGGGCACCCTGGTG
ACCGTGAGCAGCGGCGGCGGCGGCAGCGGCGGCGGCGGCAGCGGCGGCGGCGGCAGCAGCTACGTGCT
GACCCAGCCCCCCAGCGTGAGCGTGGCCCCCGGCAAGACCGCCACCATCGCCTGCGGCGGCGAGAACA
TCGGCCGCAAGACCGTGCACTGGTACCAGCAGAAGCCCGGCCAGGCCCCCGTGCTGGTGATCTACTAC
GACAGCGACCGCCCCAGCGGCATCCCCGAGCGCTTCAGCGGCAGCAACAGCGGCAACACCGCCACCCT
GACCATCAGCCGCGTGGAGGCCGGCGACGAGGCCGACTACTACTGCCTGGTGTGGGACAGCAGCAGCG
ACCACCGCATCTTCGGCGGCGGCACCAAGCTGACCGTGCTGGAGAGCAAGTACGGCCCCCCCTGCCCC
CCCTGCCCCGCCCCCGAGTTCCTGGGCGGCCCCAGCGTGTTCCTGTTCCCCCCCAAGCCCAAGGACAC
CCTGATGATCAGCCGCACCCCCGAGGTGACCTGCGTGGTGGTGGACGTGAGCCAGGAGGACCCCGAGG
TGCAGTTCAACTGGTACGTGGACGGCGTGGAGGTGCACAACGCCAAGACCAAGCCCCGCGAGGAGCAG
TTCAACAGCACCTACCGCGTGGTGAGCGTGCTGACCGTGCTGCACCAGGACTGGCTGAACGGCAAGGA
GTACAAGTGCAAGGTGAGCAACAAGGGCCTGCCCAGCAGCATCGAGAAGACCATCAGCAAGGCCAAGG
GCCAGCCCCGCGAGCCCCAGGTGTACACCCTGCCCCCCAGCCAGGAGGAGATGACCAAGAACCAGGTG
AGCCTGACCTGCCTGGTGAAGGGCTTCTACCCCAGCGACATCGCCGTGGAGTGGGAGAGCAACGGCCA
GCCCGAGAACAACTACAAGACCACCCCCCCCGTGCTGGACAGCGACGGCAGCTTCTTCCTGTACAGCC
GCCTGACCGTGGACAAGAGCCGCTGGCAGGAGGGCAACGTGTTCAGCTGCAGCGTGATGCACGAGGCC
CTGCACAACCACTACACCCAGAAGAGCCTGAGCCTGAGCCTGGGCAAGGGAGGTGGCGGGTCAGGCGG
AGGTGGTAGTGGCGGCGGTGGGAGTATTCCTCCTCACGTCCAAAAGTCAGTAAATAATGATATGATAG
TCACTGATAATAATGGTGCTGTAAAGTTCCCACAGCTTTGCAAGTTTTGCGATGTCAGGTTTTCTACC
TGCGATAATCAGAAGAGCTGCATGAGCAATTGCTCTATTACATCCATCTGTGAAAAGCCGCAGGAGGT
ATGTGTAGCAGTATGGAGAAAGAACGATGAAAACATTACACTCGAAACCGTTTGCCACGACCCAAAAC
TTCCATATCACGATTTTATACTCGAAGATGCGGCAAGTCCCAAATGCATAATGAAAGAGAAGAAAAAA
CCAGGCGAAACGTTTTTTATGTGTAGTTGCAGTAGCGATGAGTGCAACGATAATATAATCTTCTCAGA
AGAGTATAACACTTCTAACCCAGAC
SEQ ID NO: 48
Protein
Artificial
H6B1LEM anti-PD-L1 ScFv-Fc4-TGFβRII_{ecto} fusion protein
MEWSWVFLFFLSVTTGVHSQMQLVQSGAEVKKPGSSVKVSCKASGGTFSSYAYSWVRQAPGQGLEWMG
GIIPSFGTANYAQKFQGRVTITADESTSTAYMELSSLRSEDTAVYYCARGPIVATITPLDYWGQGTLV
TVSSGGGGSGGGGSGGGGSSYVLTQPPSVSVAPGKTATIACGGENIGRKTVHWYQQKPGQAPVLVIYY
DSDRPSGIPERFSGSNSGNTATLTISRVEAGDEADYYCLVWDSSSDHRIFGGGTKLTVLESKYGPPCP
PCPAPEFLGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVFSCSVMHEA
LHNHYTQKSLSLSLGKGGGGSGGGGSGGGGSIPPHVQKSVNNDMIVTDNNGAVKFPQLCKFCDVRFST
CDNQKSCMSNCSITSICEKPQEVCVAVWRKNDENITLETVCHDPKLPYHDFILEDAASPKCIMKEKKK
PGETFFMCSCSSDECNDNIIFSEEYNTSNPD
SEQ ID NO: 49
DNA
Artificial
avelumab anti-PD-L1 ScFv-Fc4-TGFβRII_{ecto} construct
ATGGAATGGTCCTGGGTCTTCCTTTTCTTCCTCTCTGTTACCACTGGCGTTCACAGCCAATCAGCACT
TACGCAACCTGCTAGCGTTAGTGGTAGTCCAGGTCAGAGCATCACAATTTCATGTACTGGGACCTCCA
GCGATGTAGGAGGATACAACTACGTGTCATGGTATCAGCAGCACCCCGGCAAGGCCCCTAAGCTTATG
ATCTACGACGTCTCAAACAGGCCCAGTGGTGTTAGTAACAGGTTTAGCGGATCAAAATCTGGAAATAC
AGCGAGTCTCACTATTTCCGGGTTGCAAGCTGAGGATGAAGCTGACTATTATTGTTCTTCATACACAT
CTTCATCAACAAGAGTATTTGGGACAGGAACAAAAGTGACAGTCTTGGGTGGTGGAGGCAGTGGGGGA
GGAGGTAGTGGCGGCGGTGGGTCAGAAGTCCAACTCTTGGAAAGCGGCGGGGGCCTCGTACAACCTGG
TGGCAGTCTGAGACTGTCATGCGCGGCAAGCGGATTCACTTTCTCATCTTATATAATGATGTGGGTTA
GACAAGCGCCAGGGAAAGGCTTGGAATGGGTAAGTTCAATCTACCCGTCTGGCGGGATTACGTTCTAC
GCTGATACGGTGAAAGGGAGGTTCACGATTTCTCGAGACAACTCCAAGAATACGCTGTATCTTCAGAT
GAACTCTCTCCGAGCCGAAGATACCGCAGTATACTACTGCGCACGCATTAAACTGGGCACCGTTACCA
CAGTTGATTACTGGGGCCAGGGTACGCTCGTGACGGTCTCTAGCGAGAGCAAGTACGGCCCCCCCTGC
CCCCCCTGCCCCGCCCCCGAGTTCCTGGGCGGCCCCAGCGTGTTCCTGTTCCCCCCCAAGCCCAAGGA
CACCCTGATGATCAGCCGCACCCCCGAGGTGACCTGCGTGGTGGTGGACGTGAGCCAGGAGGACCCCG
AGGTGCAGTTCAACTGGTACGTGGACGGCGTGGAGGTGCACAACGCCAAGACCAAGCCCCGCGAGGAG
CAGTTCAACAGCACCTACCGCGTGGTGAGCGTGCTGACCGTGCTGCACCAGGACTGGCTGAACGGCAA
GGAGTACAAGTGCAAGGTGAGCAACAAGGGCCTGCCCAGCAGCATCGAGAAGACCATCAGCAAGGCCA
AGGGCCAGCCCCGCGAGCCCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGTGGAGTGGGAGAGCAACGG
CCAGCCCGAGAACAACTACAAGACCACCCCCCCCGTGCTGGACAGCGACGGCAGCTTCTTCCTGTACA
GCCGCCTGACCGTGGACAAGAGCCGCTGGCAGGAGGGCAACGTGTTCAGCTGCAGCGTGATGCACGAG
GCCCTGCACAACCACTACACCCAGAAGAGCCTGAGCCTGAGCCTGGGCAAGGGAGGTGGCGGGTCAGG
CGGAGGTGGTAGTGGCGGCGGTGGGAGTATTCCTCCTCACGTCCAAAAGTCAGTAAATAATGATATGA
TAGTCACTGATAATAATGGTGCTGTAAAGTTCCCACAGCTTTGCAAGTTTTGCGATGTCAGGTTTTCT
ACCTGCGATAATCAGAAGAGCTGCATGAGCAATTGCTCTATTACATCCATCTGTGAAAAGCCGCAGGA
GGTATGTGTAGCAGTATGGAGAAAGAACGATGAAAACATTACACTCGAAACCGTTTGCCACGACCCAA
AACTTCCATATCACGATTTTATACTCGAAGATGCGGCAAGTCCCAAATGCATAATGAAAGAGAAGAAA
AAACCAGGCGAAACGTTTTTTATGTGTAGTTGCAGTAGCGATGAGTGCAACGATAATATAATCTTCTC
AGAAGAGTATAACACTTCTAACCCAGAC
SEQ ID NO: 50
Protein
Artificial
avelumab anti-PD-L1 ScFv-Fc4-TGFβRII_{ecto} fusion protein
MEWSWVFLFFLSVTTGVHSQSALTQPASVSGSPGQSITISCTGTSSDVGGYNYVSWYQQHPGKAPKLM
IYDVSNRPSGVSNRFSGSKSGNTASLTISGLQAEDEADYYCSSYTSSSTRVFGTGTKVTVLGGGGSGG
GGSGGGGSEVQLLESGGGLVQPGGSLRLSCAASGFTFSSYIMMWVRQAPGKGLEWVSSIYPSGGITFY
ADTVKGRFTISRDNSKNTLYLQMNSLRAEDTAVYYCARIKLGTVTTVDYWGQGTLVTVSSESKYGPPC
PPCPAPEFLGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSVMHE
ALHNHYTQKSLSLSLGKGGGGSGGGGSGGGGSIPPHVQKSVNNDMIVTDNNGAVKFPQLCKFCDVRFS
TCDNQKSCMSNCSITSICEKPQEVCVAVWRKNDENITLETVCHDPKLPYHDFILEDAASPKCIMKEKK
KPGETFFMCSCSSDECNDNIIFSEEYNTSNPD
SEQ ID NO: 51
DNA
Artificial
Encodes human IL12 (p40-2x elastin-p35)
ATGTGTCATCAGCAGCTGGTGATTTCTTGGTTCTCTTTAGTGTTTCTGGCCAGCCCTCTGGTCGCCAT
CTGGGAGCTCAAGAAGGATGTGTACGTCGTGGAGCTCGATTGGTACCCCGATGCCCCCGGTGAAATGG
TCGTGCTCACTTGTGACACCCCCGAAGAGGATGGCATCACATGGACTTTAGATCAGTCCAGCGAGGTG
CTGGGAAGCGGCAAGACTTTAACAATCCAAGTTAAGGAGTTCGGAGACGCCGGACAGTATACTTGTCA
CAAGGGCGGCGAAGTGCTGAGCCATTCTTTACTGTTATTACATAAGAAGGAGGACGGCATCTGGAGCA
CCGACATCCTCAAGGACCAGAAGGAGCCCAAAAACAAGACCTTCTTACGTTGTGAGGCCAAAAACTAT
TCCGGCAGATTTACTTGTTGGTGGCTGACAACCATCAGCACAGATTTAACCTTTAGCGTGAAGAGCTC
TCGTGGAAGCAGCGACCCTCAAGGTGTGACATGTGGAGCCGCCACCCTCAGCGCCGAAAGGGTTCGTG
GCGATAATAAAGAATACGAGTATAGCGTGGAGTGCCAAGAAGACAGCGCTTGCCCCGCTGCCGAAGAA
TCTTTACCCATCGAGGTGATGGTCGACGCCGTCCACAAGCTGAAGTACGAAAACTACACATCCTCCTT
CTTCATCAGAGATATCATCAAGCCCGATCCCCCCAAGAATCTGCAGCTGAAACCTTTAAAGAATTCTC
GTCAAGTTGAAGTGAGCTGGGAGTATCCCGACACTTGGTCCACCCCCCACTCCTACTTCAGCCTCACC
TTCTGCGTGCAAGTTCAAGGCAAATCTAAAAGGGAGAAGAAGGACAGAGTGTTTACAGACAAGACCAG
CGCTACCGTGATCTGTCGTAAGAACGCCTCCATCAGCGTGAGGGCTCAAGATCGTTATTACAGCTCCA
GCTGGAGCGAATGGGCTTCCGTGCCTTGTTCTGTGCCCGGTGTGGGCGTGCCCGGCGTGGGCAGAAAT
CTCCCCGTCGCCACCCCCGATCCCGGAATGTTCCCTTGTTTACACCATTCCCAGAATTTATTAAGGGC
CGTGAGCAACATGCTGCAAAAGGCCAGACAGACACTGGAGTTCTACCCTTGCACCAGCGAGGAGATTG
ATCACGAGGACATCACCAAGGACAAAACCAGCACAGTGGAGGCTTGTTTACCTCTGGAACTCACCAAG
AACGAGTCTTGTTTAAACTCCAGAGAGACCAGCTTTATCACCAACGGCAGCTGTTTAGCCTCTCGTAA
AACCAGCTTCATGATGGCTTTATGTTTAAGCAGCATCTACGAGGATTTAAAGATGTACCAAGTTGAAT
TCAAGACCATGAACGCCAAGTTATTAATGGATCCCAAGAGGCAGATCTTTTTAGACCAGAACATGCTG
GCCGTGATCGACGAGCTGATGCAAGCTTTAAACTTCAACTCCGAAACCGTGCCCCAGAAAAGCAGCCT
CGAGGAGCCCGACTTCTACAAAACAAAAATTAAGCTGTGCATCTTATTACACGCCTTTAGGATTCGTG
CCGTGACCATCGATCGTGTCATGAGCTATTTAAACGCTTCCTAG
SEQ ID NO: 52
protein
Artificial
Human IL12 (p40-2x elastin-p35)
MCPQKLTISWFAIVLLVSPLMAMWELEKDVYVVEVDWTPDAPGETVNLTCDTPEEDDITWTSDQRHGV
IGSGKTLTITVKEFLDAGQYTCHKGGETLSHSHLLLHKKENGIWSTEILKNFKNKTFLKCEAPNYSGR
FTCSWLVQRNMDLKFNIKSSSSSPDSRAVTCGMASLSAEKVTLDQRDYEKYSVSCQEDVTCPTAEETL
PIELALEARQQNKYENYSTSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSSCSKWACVPCRVRSVPGVG
VPGVGRVIPVSGPARCLSQSRNLLKTTDDMVKTAREKLKHYSCTAEDIDHEDITRDQTSTLKTCLPLE
LHKNESCLATRETSSTTRGSCLPPQKTSLMMTLCLGSIYEDLKMYQTEFQAINAALQNHNHQQIILDK
GMLVAIDELMQSLNHNGETLRQKPPVGEADPYRVKMKLCILLHAFSTRVVTINRVMGYLSSA
SEQ ID NO: 53
DNA
Artificial
Encodes mouse IL12 (p40-2x elastin-p35)
ATGTGTCCTCAGAAGCTGACAATTAGTTGGTTCGCCATTGTTCTCCTCGTCTCACCACTTATGGCAAT
GTGGGAACTGGAAAAAGACGTTTACGTGGTTGAGGTTGATTGGACTCCCGACGCCCCAGGTGAAACCG
TAAATCTGACCTGTGACACACCTGAGGAAGATGACATCACCTGGACCAGTGACCAGCGCCACGGAGTG
ATAGGGAGTGGGAAGACATTGACAATCACTGTAAAAGAGTTTCTCGACGCTGGACAATACACATGTCA
CAAGGGAGGCGAAACACTCTCTCATAGCCACTTGCTGTTGCACAAGAAGGAGAACGGCATATGGTCCA
CAGAGATTCTCAAGAATTTCAAGAATAAAACCTTCCTCAAGTGCGAAGCCCCTAACTATAGTGGGAGG
TTTACTTGCTCATGGCTCGTGCAGCGCAATATGGACCTGAAGTTCAACATTAAAAGTTCTAGCTCATC
CCCAGACTCACGCGCCGTGACATGTGGGATGGCAAGCCTCAGCGCCGAGAAAGTAACATTGGATCAGA
GAGACTATGAGAAATATTCCGTGAGCTGCCAAGAAGACGTTACCTGTCCAACCGCCGAGGAGACCCTG
CCTATAGAGTTGGCTCTTGAGGCAAGGCAACAAAACAAATACGAGAACTATTCCACAAGTTTTTTCAT
AAGAGACATAATCAAGCCTGACCCCCCAAAAAATCTCCAGATGAAGCCACTGAAAAATTCTCAAGTCG
AGGTTAGTTGGGAATATCCAGATTCTTGGTCAACTCCACACAGTTATTTCTCTCTTAAGTTTTTCGTT
CGCATACAGCGGAAAAAGGAGAAGATGAAGGAAACCGAGGAAGGGTGCAATCAAAAAGGAGCTTTCTT
GGTAGAGAAAACATCCACTGAAGTCCAGTGCAAAGGTGGTAACGTGTGCGTCCAGGCTCAGGATAGAT
ACTATAACTCATCCTGCTCAAAATGGGCTTGCGTCCCATGCCGCGTACGAAGCGTGCCAGGAGTAGGA
GTTCCAGGTGTTGGCCGGGTCATACCTGTAAGTGGTCCCGCTCGGTGTCTCTCTCAGTCTCGGAATCT
CCTTAAAACAACAGACGACATGGTAAAAACAGCCCGGGAGAAATTGAAACACTACTCTTGCACAGCCG
AAGACATTGATCATGAAGACATCACCCGAGACCAGACCTCAACACTGAAAACATGCCTTCCACTCGAG
CTGCACAAGAATGAATCATGCCTGGCTACTCGCGAGACAAGCAGTACTACCCGCGGTAGCTGCCTCCC
ACCCCAAAAGACATCTCTTATGATGACCCTGTGCCTCGGCTCTATCTACGAGGACCTTAAGATGTACC
AAACCGAATTCCAAGCCATCAACGCTGCATTGCAAAATCATAATCATCAACAGATAATTCTCGATAAG
GGTATGCTCGTAGCTATAGATGAGCTTATGCAGTCACTTAACCACAACGGTGAGACCTTGAGGCAAAA
ACCACCCGTGGGAGAAGCAGATCCATATCGAGTGAAGATGAAGCTCTGCATCTTGTTGCATGCCTTTA
GTACCAGGGTGGTAACTATAAACAGGGTAATGGGTTACCTCTCTTCCGCCtga
SEQ ID NO: 54
Protein
Artificial
Mouse IL12 (p40-2x elastin-p35)
MCPQKLTISWFAIVLLVSPLMAMWELEKDVYVVEVDWTPDAPGETVNLTCDTPEEDDITWTSDQRHGV
IGSGKTLTITVKEFLDAGQYTCHKGGETLSHSHLLLHKKENGIWSTEILKNFKNKTFLKCEAPNYSGR
FTCSWLVQRNMDLKFNIKSSSSSPDSRAVTCGMASLSAEKVTLDQRDYEKYSVSCQEDVTCPTAEETL
PIELALEARQQNKYENYSTSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSSCSKWACVPCRVRSVPGVG
VPGVGRVIPVSGPARCLSQSRNLLKTTDDMVKTAREKLKHYSCTAEDIDHEDITRDQTSTLKTCLPLE
LHKNESCLATRETSSTTRGSCLPPQKTSLMMTLCLGSIYEDLKMYQTEFQAINAALQNHNHQQIILDK
GMLVAIDELMQSLNHNGETLRQKPPVGEADPYRVKMKLCILLHAFSTRVVTINRVMGYLSSA
SEQ ID NO: 55
Protein
2x elastin linker
VPGVGVPGVG
SEQ ID NO: 56
Protein
(GGGGS)₃ linker
GGGGSGGGGSGGGGS
SEQ ID NO: 57
Protein
Homo sapiens
P40 subunit of IL12
MCHQQLVISWFSLVFLASPLVAIWELKKDVYVVELDWYPDAPGEMVVLTCDTPEEDGITWTLDQSSEV
LGSGKTLTIQVKEFGDAGQYTCHKGGEVLSHSLLLLHKKEDGIWSTDILKDQKEPKNKTFLRCEAKNY
SGRFTCWWLTTISTDLTFSVKSSRGSSDPQGVTCGAATLSAERVRGDNKEYEYSVECQEDSACPAAEE
SLPIEVMVDAVHKLKYENYTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEWASVPCS
SEQ ID NO: 58
Protein
Homo sapiens
P35 subunit of IL12
RNLPVATPDPGMFPCLHHSQNLLRAVSNMLQKARQTLEFYPCTSEEIDHEDITKDKTSTVEACLPLEL
TKNESCLNSRETSFITNGSCLASRKTSFMMALCLSSIYEDLKMYQVEFKTMNAKLLMDPKRQIFLDQN
MLAVIDELMQALNFNSETVPQKSSLEEPDFYKTKIKLCILLHAFRIRAVTIDRVMSYLNAS
SEQ ID NO: 59
Protein
Mus musculus
P40 subunit of IL12
MCPQKLTISWFAIVLLVSPLMAMWELEKDVYVVEVDWTPDAPGETVNLTCDTPEEDDITWTSDQRHGV
IGSGKTLTITVKEFLDAGQYTCHKGGETLSHSHLLLHKKENGIWSTEILKNFKNKTFLKCEAPNYSGR
FTCSWLVQRNMDLKFNIKSSSSSPDSRAVTCGMASLSAEKVTLDQRDYEKYSVSCQEDVTCPTAEETL
PIELALEARQQNKYENYSTSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSSCSKWACVPCRVRS
SEQ ID NO: 60
Protein
Mus musculus
P35 subunit of IL12
RVIPVSGPARCLSQSRNLLKTTDDMVKTAREKLKHYSCTAEDIDHEDITRDQTSTLKTCLPLELHKNE
SCLATRETSSTTRGSCLPPQKTSLMMTLCLGSIYEDLKMYQTEFQAINAALQNHNHQQIILDKGMLVA
IDELMQSLNHNGETLRQKPPVGEADPYRVKMKLCILLHAFSTRVVTINRVMGYLSSA

We claim:

1. A recombinant oncolytic herpes simplex virus (HSV) comprising a nucleic acid construct encoding a fusion protein comprising an ScFv that specifically binds an immune checkpoint protein, wherein the ScFv is fused to a TGFβRII ectodomain (TGFβRII_{ecto}).

2. A recombinant oncolytic HSV according to claim 1, wherein the ScFv is derived from an anti-PD-1 monoclonal antibody or anti-PD-L1 monoclonal antibody.

3. A recombinant oncolytic HSV according to claim 2, wherein the ScFv is derived from an anti-PD-1 monoclonal antibody.

4. A recombinant oncolytic HSV according to claim 3, wherein the anti-PD-1 ScFv comprises a heavy chain variable region sequence having at least 95% identity to SEQ ID NO:8 and a light chain variable region sequence having at least 95% identity to SEQ ID NO:9.

5. A recombinant oncolytic HSV according to claim 4, wherein the anti-PD-1 ScFv has at least 95% identity to SEQ ID NO:11.

6. A recombinant oncolytic HSV according to claim 3, wherein the anti-PD-1 ScFv comprises a heavy chain variable region sequence having at least 95% identity to SEQ ID NO:12 and a light chain variable region sequence having at least 95% identity to SEQ ID NO:13.

7. A recombinant oncolytic HSV according to claim 6, wherein the anti-PD-1 ScFv has at least 95% identity to SEQ ID NO:15.

8. A recombinant oncolytic HSV according to claim 3, wherein the anti-PD-1 ScFv comprises a heavy chain variable region sequence having at least 95% identity to SEQ ID NO:16 and a light chain variable region sequence having at least 95% identity to SEQ ID NO:17.

9. A recombinant oncolytic HSV according to claim 8, wherein the anti-PD-1 ScFv has at least 95% identity to SEQ ID NO:19.

10. A recombinant oncolytic HSV according to claim 2, wherein the ScFv is derived from an anti-PD-L1 monoclonal antibody.

11. A recombinant oncolytic HSV according to claim 10, wherein the anti-PD-L1 ScFv comprises a heavy chain variable region sequence having at least 95% identity to SEQ ID NO:20 and a light chain variable region sequence having at least 95% identity to SEQ ID NO:21.

12. A recombinant oncolytic HSV according to claim 11, wherein the anti-PD-L1 ScFv has at least 95% identity to SEQ ID NO:23.

13. A recombinant oncolytic HSV according to claim 10, wherein the anti-PD-L1 ScFv comprises a heavy chain variable region sequence having at least 95% identity to SEQ ID NO:24 and a light chain variable region sequence having at least 95% identity to SEQ ID NO:25.

14. A recombinant oncolytic HSV according to claim 13, wherein the anti-PD-L1 ScFv has at least 95% identity to SEQ ID NO:27.

15. A recombinant oncolytic HSV according to claim 10, wherein the anti-PD-1 ScFv comprising a heavy chain variable region sequence having at least 95% identity to SEQ ID NO:28 and a light chain variable region sequence having at least 95% identity to SEQ ID NO:29.

16. A recombinant oncolytic HSV according to claim 15, wherein the anti-PD-L1 ScFv has at least 95% identity to SEQ ID NO:31.

17. A recombinant oncolytic HSV according to claim 1, wherein the ScFv is fused to TGFβRII_{ecto} via an Fc region.

18. A recombinant oncolytic HSV according to claim 1, wherein the nucleic acid construct comprises a promoter operable in a mammalian cell operably linked to the fusion protein-encoding sequence.

19. A recombinant oncolytic HSV according to claim 18, wherein the promoter is selected from the group consisting of EF1α/HTLV, CMV, and Jet.

20. A recombinant oncolytic HSV according to any of the previous claims, wherein the nucleic acid construct comprises a sequence encoding a signal peptide 5′ of the sequence encoding the ScFv.

21. A recombinant oncolytic HSV according to any of the previous claims, wherein TGFβRII ectodomain comprises an amino acid sequence having at least 95% identity to SEQ ID NO:7.

22. A recombinant oncolytic HSV according to claim 21, wherein TGFβRII ectodomain comprises SEQ ID NO:7.

23. A recombinant oncolytic HSV according to claim 17, wherein the Fc region is an IgG1 Fc region or an IgG4 Fc region.

24. A recombinant oncolytic HSV according to claim 23, wherein the Fc region has at least 95% identity to SEQ ID NO:5.

25. A recombinant oncolytic HSV according to claim 24, wherein the Fc region comprises SEQ ID NO:5.

26. A recombinant oncolytic HSV according to claim 23, wherein the Fc region has at least 95% identity to SEQ ID NO:2.

27. A recombinant oncolytic HSV according to claim 26, wherein the Fc region comprises SEQ ID NO:2.

28. A recombinant oncolytic HSV according to any of claims 1-27, further comprising a gene encoding IL12.

29. A recombinant oncolytic HSV according to claim 28, wherein the gene encoding IL12 encodes a polypeptide having at least 90% identity to human IL12 (SEQ ID NO:52).

30. A recombinant oncolytic HSV according to claim 28, wherein the gene encoding IL12 encodes a polypeptide having at least 90% identity to murine IL12 (SEQ ID NO:54).

31. A recombinant oncolytic HSV according to any of claims 28-30, wherein the IL12 gene is operably linked to a second promoter operable in a mammalian cell.

32. A recombinant oncolytic HSV according to claim 31, wherein the promoter is selected from the group consisting of EF1α/HTLV, CMV, and Jet.

33. A recombinant oncolytic HSV comprising a nucleic acid construct encoding a fusion protein comprising an anti-PD-1 ScFv (SEQ ID NO:11) linked to TGFβRII_{ecto} (SEQ ID NO:7) via an Fc4 region (SEQ ID NO:2).

34. A recombinant oncolytic HSV according to claim 33, wherein the fusion protein comprises SEQ ID NO:40.

35. A recombinant oncolytic HSV comprising a nucleic acid construct encoding a fusion protein comprising an anti-PD-1 ScFv (SEQ ID NO:15) linked to TGFβRII_{ecto} (SEQ ID NO:7) via an Fc4 region (SEQ ID NO:2).

36. A recombinant oncolytic HSV according to claim 37, wherein the fusion protein comprises SEQ ID NO:42.

37. A recombinant oncolytic HSV comprising a nucleic acid construct encoding a fusion protein comprising an anti-PD-1 ScFv (SEQ ID NO:19) linked to TGFβRII_{ecto} (SEQ ID NO:7) via the Fc4 region (SEQ ID NO:2).

38. A recombinant oncolytic HSV according to claim 41, wherein the fusion protein comprises (SEQ ID NO:44).

39. A recombinant oncolytic HSV comprising a nucleic acid construct encoding a fusion protein comprising an anti-PD-1 ScFv (SEQ ID NO:23) linked to TGFβRII_{ecto} (SEQ ID NO:7) via an Fc4 region (SEQ ID NO:2).

40. A recombinant oncolytic HSV according to claim 33, wherein the fusion protein comprises SEQ ID NO:46.

41. A recombinant oncolytic HSV comprising a nucleic acid construct encoding a fusion protein comprising an anti-PD-1 ScFv (SEQ ID NO:27) linked to TGFβRII_{ecto} (SEQ ID NO:7) via an Fc4 region (SEQ ID NO:2).

42. A recombinant oncolytic HSV according to claim 37, wherein the fusion protein comprises SEQ ID NO:48.

43. A recombinant oncolytic HSV comprising a nucleic acid construct encoding a fusion protein comprising an anti-PD-1 ScFv (SEQ ID NO:31) linked to TGFβRII_{ecto} (SEQ ID NO:7) via the Fc4 region (SEQ ID NO:2).

44. A recombinant oncolytic HSV according to claim 41, wherein the fusion protein comprises (SEQ ID NO:50).

45. A recombinant oncolytic HSV according to any of claims 33-44, further comprising a gene encoding human IL12.

46. A recombinant oncolytic HSV according to claim 45, wherein the gene encoding human IL12 encodes the polypeptide of SEQ ID NO:52 or a polypeptide having at least 95% identity thereto.

47. A recombinant oncolytic HSV according to any of the previous claims, wherein the oncolytic HSV is an HSV-1.

48. A recombinant oncolytic HSV according to claim 33, wherein the oncolytic HSV is derived from HSV-1 strain 17, HSV-1 strain F, HSV-1 strain KOS, or HSV-1 strain JS1.

49. A recombinant oncolytic HSV according to claim 34, wherein the oncolytic HSV is derived from HSV strain 17.

50. A recombinant oncolytic HSV according to any of the previous claims, wherein the oncolytic HSV does not encode a functional ICP34.5-encoding gene.

51. A recombinant oncolytic HSV according to claim 36, wherein all or a portion of the ICP34.5-encoding gene is deleted.

52. A recombinant oncolytic HSV according to claim 36 or 37, wherein the nucleic acid construct encoding the fusion protein and/or the gene encoding IL12 are inserted into the ICP34.5-encoding gene locus.

53. A recombinant oncolytic HSV for use in a method of treating cancer, wherein the method comprises administering an oncolytic HSV according to any of claims 1-38 to a subject having cancer.

54. A recombinant oncolytic HSV according to claim 53, for use in a method comprising administering the oncolytic HSV by intravenous, intracavitary, intraperitoneal, intratumoral, or peritumoral delivery.

55. A recombinant oncolytic HSV according to claim 53 or 54, wherein the method comprises administering more than one dose of the oncolytic HSV to the patient.

56. A recombinant oncolytic HSV according to any of claims 53-55, wherein the cancer is a solid tumor.

57. A recombinant oncolytic HSV according to any of claims 53-56, wherein the subject is a human.

58. A recombinant oncolytic HSV according to any of claims 53-56, wherein the subject is a dog.

59. A pharmaceutical composition comprising a recombinant oncolytic HSV according to any of claims 1-58.

60. A pharmaceutical composition according to claim 59, wherein the oncolytic HSV is at a concentration of at least 10⁶ per ml.

61. A pharmaceutical composition according to claim 60, wherein the oncolytic HSV is at a concentration of at least 10⁷ per ml.

62. A method of treating cancer in a subject, comprising administering an oncolytic HSV or pharmaceutical composition according to any of claims 1-61 to a subject having cancer.

63. A method according to claim 62, wherein the subject is a human.

64. A method according to claim 62, wherein the subject is a dog.

65. A method according to claim 63 or 64, comprising administering the oncolytic HSV by intravenous, intra-arterial, intracavitary, intratumoral, or peritumoral delivery.

66. A method according to claim 65, comprising administering more than one dose of the oncolytic HSV to the subject.

67. A method according to any of claims 60-66, wherein the cancer is a solid tumor.

68. A fusion protein comprising a single chain variable fragment (ScFv) that binds an immune checkpoint protein, a TGFβRII_{ecto} ectodomain (TGFβRII_{ecto}), and an Fc antibody region linking the ScFv to the TGFβRII_{ecto}.

69. A fusion protein according to claim 68, wherein the immune checkpoint protein is PD-1 or PD-L1.

70. A fusion protein according to claim 69, wherein the immune checkpoint protein is PD-1.

71. A fusion protein according to claim 70, wherein the ScFv is derived from a BB9 anti-PD-1 monoclonal antibody, an RG1H10 anti-PD-1 monoclonal antibody, or pembrolizumab.

72. A fusion protein according to claim 71, wherein the ScFv comprises a sequence having at least 95% identity to SEQ ID NO: 11, SEQ ID NO:15, or SEQ ID NO:19.

73. A fusion protein according to claim 69, wherein the immune checkpoint protein is PD-L1.

74. A fusion protein according to claim 73, wherein the ScFv is derived from a Combi5 anti-PD-L1 monoclonal antibody, an H6B1LEM anti-PD-L1 monoclonal antibody, or avelumab.

75. A fusion protein according to claim 74, wherein the ScFv comprises a sequence having at least 95% identity to SEQ ID NO:23, SEQ ID NO:27, or SEQ ID NO:31.

76. A fusion protein according to any of claims 68-75, wherein the TGFβRII_{ecto} comprises an amino acid sequence having at least 95% identity to SEQ ID NO:7.

77. A fusion protein according to claim 76, wherein the TGFβRII_{ecto} comprises SEQ ID NO:7.

78. A fusion protein according to any of claims 68-77, wherein the Fc is an IgG1 Fc or an IgG4 Fc.

79. A fusion protein according to claim 68, wherein the Fc is a human Fc.

80. A fusion protein according to claim 79, wherein the Fc comprises an amino acid sequence having at least 95% identity to SEQ ID NO:2 or SEQ ID NO:5.

81. A conditioned media composition comprising a fusion protein according to any of claims 68-80.

82. A conditioned media composition according to claim 81, wherein the cell supernatant is virus-free.

83. A pharmaceutical composition comprising a fusion protein according to any of claims 68-80.

84. A method of treating cancer, comprising administering a pharmaceutical composition according to claim 83 to a subject having cancer.

85. A method according to claim 84, wherein the subject is a human.

86. A method according to claim 84, wherein the subject is a dog.

87. A nucleic acid construct comprising a nucleic acid sequence encoding a fusion protein according to any of claims 68-80.

88. A nucleic acid construct according to claim 87, wherein the nucleic acid sequence encoding a fusion protein is operably linked to a promoter.

89. A nucleic acid construct according to claim 88, wherein the promoter is a eukaryotic promoter.

90. A nucleic acid construct according to claim 89, wherein the promoter is operable in a mammalian cell.


SEQUENCE LISTING (68 sequences)

SEQ ID NO: 1 - DNA, 687 bp, Artificial Sequence
  source: /note=“Description of Artificial Sequence Synthetic polynucleotide”
gagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 2 - protein, 229 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - DNA, 696 bp, Homo sapiens
gaaccaaagtcctctgataaaacacatacttgcccaccttgtcctgcaccagagctgctgggaggtccaagcgtgttcctgtttcctcccaagccaaaagatactcttatgattagccgaacaccagaggtgacgtgcgtggtggtcgacgtatcacacgaggaccctgaagtgaagtttaattggtacgtagacggggttgaggtgcataacgcgaagaccaaacctagagaggagcagtataattcaacctaccgggtcgtttctgtcctcacagtcctccaccaagattggttgaacggaaaagaatataagtgtaaagtgtctaacaaggctcttcccgcgcctatagagaaaacgatcagcaaagcgaaggggcaaccaagggaaccgcaagtctacactcttccaccgtcacgggatgagctgactaagaatcaagtgtcacttacttgccttgtaaaaggattctacccatcagacatcgcggtcgagtgggaatctaacggtcaaccggagaacaattataaaaccactcctcccgttcttgactccgacggttcatttttcctctattccaaactgacggttgataagtctcgatggcaacaaggcaatgtgtttagttgttctgtcatgcacgaagcactgcacaaccattatacacaaaagtcactttctctcagtcccggaaag

SEQ ID NO: 5 - protein, 232 aa, Artificial Sequence
  source: /note=“Description of Artificial Sequence Synthetic polypeptide”
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 408 bp, Homo sapiens
attcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 7 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 117 aa, Artificial Sequence
  source: /note=“Description of Artificial Sequence Synthetic polypeptide”
QVQLVQSGAEVKKPGASVKVSCKASGFRLTTNGISWVRQAPGQGLEWMGWISAGGGPTNYAQKLQGRVTMTTDTSTSTAYMELRSLRSDDTAVYYCAKGLYGTKDAWGQGTLVTVSS

SEQ ID NO: 9 - protein, 110 aa, Artificial Sequence
  source: /note=“Description of Artificial Sequence Synthetic polypeptide”
QSVLTQPPSVSEVPGQRVTISCSGGGSNIGSNAVNWYQHFPGKAPKLLIYYNDLLPSGVSDRFSASKSGTSASLAISGLRSEDEADYYCAAWDDNLSAYVFATGTKVTVL

SEQ ID NO: 10 - DNA, 783 bp, Artificial Sequence
  source: /note=“Description of Artificial Sequence Synthetic polynucleotide”
atggagtggagctgggtgttcctgttcttcctgagcgtgaccaccggcgtgcacagccaggtgcagctggtgcagagcggcgccgaggtgaagaagcccggcgccagcgtgaaggtgagctgcaaggccagcggcttccgcctgaccaccaacggcatcagctgggtgcgccaggcccccggccagggcctggagtggatgggctggatcagcgccggcggcggccccaccaactacgcccagaagctgcagggccgcgtgaccatgaccaccgacaccagcaccagcaccgcctacatggagctgcgcagcctgcgcagcgacgacaccgccgtgtactactgcgccaagggcctgtacggcaccaaggacgcctggggccagggcaccctggtgaccgtgagcagcggcggcggcggcagcggcggcggcggcagcggcggcggcggcagccagagcgtgctgacccagccccccagcgtgagcgaggtgcccggccagcgcgtgaccatcagctgcagcggcggcggcagcaacatcggcagcaacgccgtgaactggtaccagcacttccccggcaaggcccccaagctgctgatctactacaacgacctgctgcccagcggcgtgagcgaccgcttcagcgccagcaagagcggcaccagcgccagcctggccatcagcggcctgcgcagcgaggacgaggccgactactactgcgccgcctgggacgacaacctgagcgcctacgtgttcgccaccggcaccaaggtgaccgtgctg

SEQ ID NO: 11 - protein, 261 aa, Homo sapiens
MEWSWVFLFFLSVTTGVHSQVQLVQSGAEVKKPGASVKVSCKASGFRLTTNGISWVRQAPGQGLEWMGWISAGGGPTNYAQKLQGRVTMTTDTSTSTAYMELRSLRSDDTAVYYCAKGLYGTKDAWGQGTLVTVSSGGGGSGGGGSGGGGSQSVLTQPPSVSEVPGQRVTISCSGGGSNIGSNAVNWYQHFPGKAPKLLIYYNDLLPSGVSDRFSASKSGTSASLAISGLRSEDEADYYCAAWDDNLSAYVFATGTKVTVL

SEQ ID NO: 12 - protein, 119 aa, Artificial Sequence
  source: /note=“Description of Artificial Sequence Synthetic polypeptide”
QVQLVQSGSELKKPGASVKISCKASGYIFSDNGVNWVRQAPGQGLEWMGWINTKIGNPTYAQGFTGRFVFSLDTSISTTYLQISSLQAGDTAVYYCAREHDYYYGMDVWGQGTTVTVSS

SEQ ID NO: 13 - protein, 111 aa, Artificial Sequence
  source: /note=“Description of Artificial Sequence Synthetic polypeptide”
QSALTQPPSASGSPGQSVTISCTGTSSDVGGYNYVSWYQHHPGKAPKLMIYEVSKRPSGVPDRFSGSKSAITASLTISGLLTEDEADYYCSAWDDSLNADVFGGGTKVTVL

SEQ ID NO: 14 - DNA, 792 bp, Artificial Sequence
  source: /note=“Description of Artificial Sequence Synthetic polynucleotide”
atggagtggtcttgggtattcctcttttttcttagcgtcaccacaggtgttcactcccaagttcaacttgtgcaaagcggctccgagttgaagaaacctggggcaagcgtgaagatctcatgcaaagcttccggctatatattttcagacaatggggttaactgggtacgccaggccccgggtcagggactggagtggatgggttggatcaatacaaagattggtaatcctacatacgcacagggatttacggggcgattcgtattctcactcgatacctctataagcacaacctaccttcagataagctccttgcaagcaggtgataccgctgtgtactactgcgcacgggaacacgactattattacggtatggatgtgtggggtcaagggaccacagtgactgttagcagcggcggcggaggatccggaggcggaggaagtggtggcgggggttcacaaagcgctctcacacaaccaccaagtgccagtgggtccccaggacaaagtgttacaatctcctgcactgggacctcatctgatgtgggggggtacaattatgtaagctggtaccaacatcatccaggaaaggctccaaaattgatgatctatgaagtgtctaagcggccttcaggagttccggacaggttctctgggtcaaaaagtgccatcacggcgtcacttacgatttctggcctccttaccgaagatgaagccgactactactgctcagcatgggacgattccctgaacgcggatgtattcgggggtggcaccaaagttacggtcctg

SEQ ID NO: 15 - protein, 264 aa, Artificial Sequence
  source: /note=“Description of Artificial Sequence Synthetic polypeptide”
MEWSWVFLFFLSVTTGVHSQVQLVQSGSELKKPGASVKISCKASGYIFSDNGVNWVRQAPGQGLEWMGWINTKIGNPTYAQGFTGRFVFSLDTSISTTYLQISSLQAGDTAVYYCAREHDYYYGMDVWGQGTTVTVSSGGGGSGGGGSGGGGSQSALTQPPSASGSPGQSVTISCTGTSSDVGGYNYVSWYQHHPGKAPKLMIYEVSKRPSGVPDRFSGSKSAITASLTISGLLTEDEADYYCSAWDDSLNADVFGGGTKVTVL

SEQ ID NO: 16 - protein, 120 aa, Artificial Sequence [sequence omitted]

SEQ ID NO: 17 - protein, 119 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKTSENLYFQ

SEQ ID NO: 18 - DNA, 795 bp, Artificial Sequence
  source: /note=“Description of Artificial Sequence Synthetic polynucleotide”
atggagtggagttgggtctttctcttcttcctgtctgtaacgactggcgtgcattcacaggtgcaacttgtacagagcggcgttgaagtgaaaaaacccggcgcaagtgtgaaagtcagctgcaaagcgtcaggctacacgttcacgaattattacatgtattgggttaggcaggcacctggacaggggctggagtggatgggtggcataaacccttctaatggcggaaccaactttaacgagaagtttaagaaccgagtaacactcacgactgatagcagtacgaccacggcgtacatggaacttaaaagcctccaatttgacgatacagctgtgtactattgcgccagacgcgattaccggttcgacatgggcttcgactattggggtcagggaacgacggtcacagtcagttctgggggaggaggtagtggagggggagggagtgggggcggaggtagtgagatagttttgacgcagtccccggcaactctgtccctgtcacctggtgaaagggccaccctgagctgccgggcgtcaaaaggggtatccacgagcggatattcctatttgcattggtaccagcaaaaacccggtcaggctccgaggcttttgatttacttggcgtcctatctggaaagcggagttcccgctcgcttttcaggctccggtagcggtacagattttactctcacgatttcttccctggagccggaagactttgcggtatattattgtcagcatagtcgcgacctccctcttacattcggaggaggtacgaaggtcgaaattaaa

SEQ ID NO: 19 - protein, 265 aa, Artificial Sequence
  source: /note=“Description of Artificial Sequence Synthetic polypeptide”
MEWSWVFLFFLSVTTGVHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 20 - protein, 124 aa, Artificial Sequence
  source: /note=“Description of Artificial Sequence Synthetic polypeptide”
QVQLVQSGAEVKKPGASVKVSCKTSGNTFTNYALHWVRQAPGQGLEWMGGMKPSGGSTSIAQKFQGRVTMTRDKSTSTVYMELSSLTSEDTAVYYCARDLFPHIFGNYYGMDIWGQGTTVTVSS

SEQ ID NO: 21 - protein, 107 aa, Artificial Sequence
  source: /note=“Description of Artificial Sequence Synthetic polypeptide”
DIVMTQSPPSLSASVGDRVTITCRASQSISSYLNWYQQKPGKAPKLLIYATSSLQYGVPSRFSGSGSGTDFTLTISSLQPEDFATYYCQGSYSTPYTFGQGTKVEIK

SEQ ID NO: 22 - DNA, 795 bp, Artificial Sequence
  source: /note=“Description of Artificial Sequence Synthetic polynucleotide”
atggagtggagctgggtgttcctgttcttcctgagcgtgaccaccggcgtgcacagccaggtgcagctggtgcagagcggcgccgaggtgaagaagcccggcgccagcgtgaaggtgagctgcaagaccagcggcaacaccttcaccaactacgccctgcactgggtgcgccaggcccccggccagggcctggagtggatgggcggcatgaagcccagcggcggcagcaccagcatcgcccagaagttccagggccgcgtgaccatgacccgcgacaagagcaccagcaccgtgtacatggagctgagcagcctgaccagcgaggacaccgccgtgtactactgcgcccgcgacctgttcccccacatcttcggcaactactacggcatggacatctggggccagggcaccaccgtgaccgtgagcagcggcggcggcggcagcggcggcggcggcagcggcggcggcggcagcgacatcgtgatgacccagagcccccccagcctgagcgccagcgtgggcgaccgcgtgaccatcacctgccgcgccagccagagcatcagcagctacctgaactggtaccagcagaagcccggcaaggcccccaagctgctgatctacgccaccagcagcctgcagtacggcgtgcccagccgcttcagcggcagcggcagcggcaccgacttcaccctgaccatcagcagcctgcagcccgaggacttcgccacctactactgccagggcagctacagcaccccctacaccttcggccagggcaccaaggtggagatcaag

SEQ ID NO: 23 - protein, 265 aa, Artificial Sequence
  source: /note=“Description of Artificial Sequence Synthetic polypeptide”
MEWSWVFLFFLSVTTGVHSQVQLVQSGAEVKKPGASVKVSCKTSGNTFTNYALHWVRQAPGQGLEWMGGMKPSGGSTSIAQKFQGRVTMTRDKSTSTVYMELSSLTSEDTAVYYCARDLFPHIFGNYYGMDIWGQGTTVTVSSGGGGSGGGGSGGGGSDIVMTQSPPSLSASVGDRVTITCRASQSISSYLNWYQQKPGKAPKLLIYATSSLQYGVPSRFSGSGSGTDFTLTISSLQPEDFATYYCQGSYSTPYTFGQGTKVEIK

SEQ ID NO: 24 - protein, 121 aa, Artificial Sequence
  source: /note=“Description of Artificial Sequence Synthetic polypeptide”
QMQLVQSGAEVKKPGSSVKVSCKASGGTFSSYAYSWVRQAPGQGLEWMGGIIPSFGTANYAQKFQGRVTITADESTSTAYMELSSLRSEDTAVYYCARGPIVATITPLDYWGQGTLVTVSS

SEQ ID NO: 25 - protein, 108 aa, Artificial Sequence
  source: /note=“Description of Artificial Sequence Synthetic polypeptide”
SYVLTQPPSVSVAPGKTATIACGGENIGRKTVHWYQQKPGQAPVLVIYYDSDRPSGIPERFSGSNSGNTATLTISRVEAGDEADYYCLVWDSSSDHRIFGGGTKLTVL

SEQ ID NO: 26 - DNA, 789 bp, Artificial Sequence
  source: /note=“Description of Artificial Sequence Synthetic polynucleotide”
atggagtggagctgggtgttcctgttcttcctgagcgtgaccaccggcgtgcacagccagatgcagctggtgcagagcggcgccgaggtgaagaagcccggcagcagcgtgaaggtgagctgcaaggccagcggcggcaccttcagcagctacgcctacagctgggtgcgccaggcccccggccagggcctggagtggatgggcggcatcatccccagcttcggcaccgccaactacgcccagaagttccagggccgcgtgaccatcaccgccgacgagagcaccagcaccgcctacatggagctgagcagcctgcgcagcgaggacaccgccgtgtactactgcgcccgcggccccatcgtggccaccatcacccccctggactactggggccagggcaccctggtgaccgtgagcagcggcggcggcggcagcggcggcggcggcagcggcggcggcggcagcagctacgtgctgacccagccccccagcgtgagcgtggcccccggcaagaccgccaccatcgcctgcggcggcgagaacatcggccgcaagaccgtgcactggtaccagcagaagcccggccaggcccccgtgctggtgatctactacgacagcgaccgccccagcggcatccccgagcgcttcagcggcagcaacagcggcaacaccgccaccctgaccatcagccgcgtggaggccggcgacgaggccgactactactgcctggtgtgggacagcagcagcgaccaccgcatcttcggcggcggcaccaagctgaccgtgctg

SEQ ID NO: 27 - protein, 263 aa, Artificial Sequence
  source: /note=“Description of Artificial Sequence Synthetic polypeptide”
MEWSWVFLFFLSVTTGVHSQMQLVQSGAEVKKPGSSVKVSCKASGGTFSSYAYSWVRQAPGQGLEWMGGIIPSFGTANYAQKFQGRVTITADESTSTAYMELSSLRSEDTAVYYCARGPIVATITPLDYWGQGTLVTVSSGGGGSGGGGSGGGGSSYVLTQPPSVSVAPGKTATIACGGENIGRKTVHWYQQKPGQAPVLVIYYDSDRPSGIPERFSGSNSGNTATLTISRVEAGDEADYYCLVWDSSSDHRIFGGGTKLTVL

SEQ ID NO: 28 - protein, 121 aa, Artificial Sequence
  source: /note=“Description of Artificial Sequence Synthetic polypeptide”
SEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 29 - protein, 110 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 30 - DNA, 792 bp, Artificial Sequence
  source: /note=“Description of Artificial Sequence Synthetic polynucleotide”
atggaatggtcctgggtcttccttttcttcctctctgttaccactggcgttcacagccaatcagcacttacgcaacctgctagcgttagtggtagtccaggtcagagcatcacaatttcatgtactgggacctccagcgatgtaggaggatacaactacgtgtcatggtatcagcagcaccccggcaaggcccctaagcttatgatctacgacgtctcaaacaggcccagtggtgttagtaacaggtttagcggatcaaaatctggaaatacagcgagtctcactatttccgggttgcaagctgaggatgaagctgactattattgttcttcatacacatcttcatcaacaagagtatttgggacaggaacaaaagtgacagtcttgggtggtggaggcagtgggggaggaggtagtggcggcggtgggtcagaagtccaactcttggaaagcggcgggggcctcgtacaacctggtggcagtctgagactgtcatgcgcggcaagcggattcactttctcatcttatataatgatgtgggttagacaagcgccagggaaaggcttggaatgggtaagttcaatctacccgtctggcgggattacgttctacgctgatacggtgaaagggaggttcacgatttctcgagacaactccaagaatacgctgtatcttcagatgaactctctccgagccgaagataccgcagtatactactgcgcacgcattaaactgggcaccgttaccacagttgattactggggccagggtacgctcgtgacggtctctagc

SEQ ID NO: 31 - protein, 264 aa, Artificial Sequence
  source: /note=“Description of Artificial Sequence Synthetic polypeptide”
MEWSWVFLFFLSVTTGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 32 - DNA, 525 bp, Artificial Sequence
  source: /note=“Description of Artificial Sequence Synthetic polynucleotide”
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 33 - DNA, 603 bp, Unknown
  source: /note=“Description of Unknown Cytomegalovirus sequence”
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactaga

SEQ ID NO: 34 - protein, 19 aa, Mus musculus
MEWSWVFLFFLSVTTGVHS

SEQ ID NO: 35 - protein, 20 aa, Artificial Sequence
  source: /note=“Description of Artificial Sequence Synthetic peptide”
MEFGLSWVFLVALFRGVQCD

SEQ ID NO: 36 - protein, 16 aa, Artificial Sequence
  source: /note=“Description of Artificial Sequence Synthetic peptide”
METDTLLLWVLLLWVP

SEQ ID NO: 37 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - DNA, 237 bp, Simian virus 40
tagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 39 - DNA, 1923 bp, Artificial Sequence
  source: /note=“Description of Artificial Sequence Synthetic polynucleotide”
atggagtggagctgggtgttcctgttcttcctgagcgtgaccaccggcgtgcacagccaggtgcagctggtgcagagcggcgccgaggtgaagaagcccggcgccagcgtgaaggtgagctgcaaggccagcggcttccgcctgaccaccaacggcatcagctgggtgcgccaggcccccggccagggcctggagtggatgggctggatcagcgccggcggcggccccaccaactacgcccagaagctgcagggccgcgtgaccatgaccaccgacaccagcaccagcaccgcctacatggagctgcgcagcctgcgcagcgacgacaccgccgtgtactactgcgccaagggcctgtacggcaccaaggacgcctggggccagggcaccctggtgaccgtgagcagcggcggcggcggcagcggcggcggcggcagcggcggcggcggcagccagagcgtgctgacccagccccccagcgtgagcgaggtgcccggccagcgcgtgaccatcagctgcagcggcggcggcagcaacatcggcagcaacgccgtgaactggtaccagcacttccccggcaaggcccccaagctgctgatctactacaacgacctgctgcccagcggcgtgagcgaccgcttcagcgccagcaagagcggcaccagcgccagcctggccatcagcggcctgcgcagcgaggacgaggccgactactactgcgccgcctgggacgacaacctgagcgcctacgtgttcgccaccggcaccaaggtgaccgtgctggagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 40 - protein, 641 aa, Artificial Sequence
  source: /note=“Description of Artificial Sequence Synthetic polypeptide”
MEWSWVFLFFLSVTTGVHSQVQLVQSGAEVKKPGASVKVSCKASGFRLTTNGISWVRQAPGQGLEWMGWISAGGGPTNYAQKLQGRVTMTTDTSTSTAYMELRSLRSDDTAVYYCAKGLYGTKDAWGQGTLVTVSSGGGGSGGGGSGGGGSQSVLTQPPSVSEVPGQRVTISCSGGGSNIGSNAVNWYQHFPGKAPKLLIYYNDLLPSGVSDRFSASKSGTSASLAISGLRSEDEADYYCAAWDDNLSAYVFATGTKVTVLESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 41 - DNA, 1932 bp, Artificial Sequence
  source: /note=“Description of Artificial Sequence Synthetic polynucleotide”
atggagtggtcttgggtattcctcttttttcttagcgtcaccacaggtgttcactcccaagttcaacttgtgcaaagcggctccgagttgaagaaacctggggcaagcgtgaagatctcatgcaaagcttccggctatatattttcagacaatggggttaactgggtacgccaggccccgggtcagggactggagtggatgggttggatcaatacaaagattggtaatcctacatacgcacagggatttacggggcgattcgtattctcactcgatacctctataagcacaacctaccttcagataagctccttgcaagcaggtgataccgctgtgtactactgcgcacgggaacacgactattattacggtatggatgtgtggggtcaagggaccacagtgactgttagcagcggcggcggaggatccggaggcggaggaagtggtggcgggggttcacaaagcgctctcacacaaccaccaagtgccagtgggtccccaggacaaagtgttacaatctcctgcactgggacctcatctgatgtgggggggtacaattatgtaagctggtaccaacatcatccaggaaaggctccaaaattgatgatctatgaagtgtctaagcggccttcaggagttccggacaggttctctgggtcaaaaagtgccatcacggcgtcacttacgatttctggcctccttaccgaagatgaagccgactactactgctcagcatgggacgattccctgaacgcggatgtattcgggggtggcaccaaagttacggtcctggagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 42 - protein, 644 aa, Artificial Sequence
  source: /note=“Description of Artificial Sequence Synthetic polypeptide”
MEWSWVFLFFLSVTTGVHSQVQLVQSGSELKKPGASVKISCKASGYIFSDNGVNWVRQAPGQGLEWMGWINTKIGNPTYAQGFTGRFVFSLDTSISTTYLQISSLQAGDTAVYYCAREHDYYYGMDVWGQGTTVTVSSGGGGSGGGGSGGGGSQSALTQPPSASGSPGQSVTISCTGTSSDVGGYNYVSWYQHHPGKAPKLMIYEVSKRPSGVPDRFSGSKSAITASLTISGLLTEDEADYYCSAWDDSLNADVFGGGTKVTVLESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 43 - DNA, 1935 bp, Artificial Sequence
  source: /note=“Description of Artificial Sequence Synthetic polynucleotide”
atggagtggagttgggtctttctcttcttcctgtctgtaacgactggcgtgcattcacaggtgcaacttgtacagagcggcgttgaagtgaaaaaacccggcgcaagtgtgaaagtcagctgcaaagcgtcaggctacacgttcacgaattattacatgtattgggttaggcaggcacctggacaggggctggagtggatgggtggcataaacccttctaatggcggaaccaactttaacgagaagtttaagaaccgagtaacactcacgactgatagcagtacgaccacggcgtacatggaacttaaaagcctccaatttgacgatacagctgtgtactattgcgccagacgcgattaccggttcgacatgggcttcgactattggggtcagggaacgacggtcacagtcagttctgggggaggaggtagtggagggggagggagtgggggcggaggtagtgagatagttttgacgcagtccccggcaactctgtccctgtcacctggtgaaagggccaccctgagctgccgggcgtcaaaaggggtatccacgagcggatattcctatttgcattggtaccagcaaaaacccggtcaggctccgaggcttttgatttacttggcgtcctatctggaaagcggagttcccgctcgcttttcaggctccggtagcggtacagattttactctcacgatttcttccctggagccggaagactttgcggtatattattgtcagcatagtcgcgacctccctcttacattcggaggaggtacgaaggtcgaaattaaagagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 44 - protein, 645 aa, Artificial Sequence
  source: /note=“Description of Artificial Sequence Synthetic polypeptide”
MEWSWVFLFFLSVTTGVHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 45 - DNA, 1935 bp, Artificial Sequence
  source: /note=“Description of Artificial Sequence Synthetic polynucleotide”
atggagtggagctgggtgttcctgttcttcctgagcgtgaccaccggcgtgcacagccaggtgcagctggtgcagagcggcgccgaggtgaagaagcccggcgccagcgtgaaggtgagctgcaagaccagcggcaacaccttcaccaactacgccctgcactgggtgcgccaggcccccggccagggcctggagtggatgggcggcatgaagcccagcggcggcagcaccagcatcgcccagaagttccagggccgcgtgaccatgacccgcgacaagagcaccagcaccgtgtacatggagctgagcagcctgaccagcgaggacaccgccgtgtactactgcgcccgcgacctgttcccccacatcttcggcaactactacggcatggacatctggggccagggcaccaccgtgaccgtgagcagcggcggcggcggcagcggcggcggcggcagcggcggcggcggcagcgacatcgtgatgacccagagcccccccagcctgagcgccagcgtgggcgaccgcgtgaccatcacctgccgcgccagccagagcatcagcagctacctgaactggtaccagcagaagcccggcaaggcccccaagctgctgatctacgccaccagcagcctgcagtacggcgtgcccagccgcttcagcggcagcggcagcggcaccgacttcaccctgaccatcagcagcctgcagcccgaggacttcgccacctactactgccagggcagctacagcaccccctacaccttcggccagggcaccaaggtggagatcaaggagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 46 - protein, 645 aa, Artificial Sequence
  source: /note=“Description of Artificial Sequence Synthetic polypeptide”
MEWSWVFLFFLSVTTGVHSQVQLVQSGAEVKKPGASVKVSCKTSGNTFTNYALHWVRQAPGQGLEWMGGMKPSGGSTSIAQKFQGRVTMTRDKSTSTVYMELSSLTSEDTAVYYCARDLFPHIFGNYYGMDIWGQGTTVTVSSGGGGSGGGGSGGGGSDIVMTQSPPSLSASVGDRVTITCRASQSISSYLNWYQQKPGKAPKLLIYATSSLQYGVPSRFSGSGSGTDFTLTISSLQPEDFATYYCQGSYSTPYTFGQGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 47 - DNA, 1929 bp, Artificial Sequence
  source: /note=“Description of Artificial Sequence Synthetic polynucleotide”
atggagtggagctgggtgttcctgttcttcctgagcgtgaccaccggcgtgcacagccagatgcagctggtgcagagcggcgccgaggtgaagaagcccggcagcagcgtgaaggtgagctgcaaggccagcggcggcaccttcagcagctacgcctacagctgggtgcgccaggcccccggccagggcctggagtggatgggcggcatcatccccagcttcggcaccgccaactacgcccagaagttccagggccgcgtgaccatcaccgccgacgagagcaccagcaccgcctacatggagctgagcagcctgcgcagcgaggacaccgccgtgtactactgcgcccgcggccccatcgtggccaccatcacccccctggactactggggccagggcaccctggtgaccgtgagcagcggcggcggcggcagcggcggcggcggcagcggcggcggcggcagcagctacgtgctgacccagccccccagcgtgagcgtggcccccggcaagaccgccaccatcgcctgcggcggcgagaacatcggccgcaagaccgtgcactggtaccagcagaagcccggccaggcccccgtgctggtgatctactacgacagcgaccgccccagcggcatccccgagcgcttcagcggcagcaacagcggcaacaccgccaccctgaccatcagccgcgtggaggccggcgacgaggccgactactactgcctggtgtgggacagcagcagcgaccaccgcatcttcggcggcggcaccaagctgaccgtgctggagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 48 - protein, 643 aa, Artificial Sequence
  source: /note=“Description of Artificial Sequence Synthetic polypeptide”
MEWSWVFLFFLSVTTGVHSQMQLVQSGAEVKKPGSSVKVSCKASGGTFSSYAYSWVRQAPGQGLEWMGGIIPSFGTANYAQKFQGRVTITADESTSTAYMELSSLRSEDTAVYYCARGPIVATITPLDYWGQGTLVTVSSGGGGSGGGGSGGGGSSYVLTQPPSVSVAPGKTATIACGGENIGRKTVHWYQQKPGQAPVLVIYYDSDRPSGIPERFSGSNSGNTATLTISRVEAGDEADYYCLVWDSSSDHRIFGGGTKLTVLESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 49 - DNA, 1932 bp, Artificial Sequence
  source: /note=“Description of Artificial Sequence Synthetic polynucleotide”
atggaatggtcctgggtcttccttttcttcctctctgttaccactggcgttcacagccaatcagcacttacgcaacctgctagcgttagtggtagtccaggtcagagcatcacaatttcatgtactgggacctccagcgatgtaggaggatacaactacgtgtcatggtatcagcagcaccccggcaaggcccctaagcttatgatctacgacgtctcaaacaggcccagtggtgttagtaacaggtttagcggatcaaaatctggaaatacagcgagtctcactatttccgggttgcaagctgaggatgaagctgactattattgttcttcatacacatcttcatcaacaagagtatttgggacaggaacaaaagtgacagtcttgggtggtggaggcagtgggggaggaggtagtggcggcggtgggtcagaagtccaactcttggaaagcggcgggggcctcgtacaacctggtggcagtctgagactgtcatgcgcggcaagcggattcactttctcatcttatataatgatgtgggttagacaagcgccagggaaaggcttggaatgggtaagttcaatctacccgtctggcgggattacgttctacgctgatacggtgaaagggaggttcacgatttctcgagacaactccaagaatacgctgtatcttcagatgaactctctccgagccgaagataccgcagtatactactgcgcacgcattaaactgggcaccgttaccacagttgattactggggccagggtacgctcgtgacggtctctagcgagagcaagtacggccccccctgccccccctgccccgcccccgagttcctgggcggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaagggaggtggcgggtcaggcggaggtggtagtggcggcggtgggagtattcctcctcacgtccaaaagtcagtaaataatgatatgatagtcactgataataatggtgctgtaaagttcccacagctttgcaagttttgcgatgtcaggttttctacctgcgataatcagaagagctgcatgagcaattgctctattacatccatctgtgaaaagccgcaggaggtatgtgtagcagtatggagaaagaacgatgaaaacattacactcgaaaccgtttgccacgacccaaaacttccatatcacgattttatactcgaagatgcggcaagtcccaaatgcataatgaaagagaagaaaaaaccaggcgaaacgttttttatgtgtagttgcagtagcgatgagtgcaacgataatataatcttctcagaagagtataacacttctaacccagac

SEQ ID NO: 50 - protein, 644 aa, Artificial Sequence
  source: /note=“Description of Artificial Sequence Synthetic polypeptide”
MEWSWVFLFFLSVTTGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 51 - DNA, 1608 bp, Artificial Sequence
  source: /note=“Description of Artificial Sequence Synthetic polynucleotide”
atgtgtcatcagcagctggtgatttcttggttctctttagtgtttctggccagccctctggtcgccatctgggagctcaagaaggatgtgtacgtcgtggagctcgattggtaccccgatgcccccggtgaaatggtcgtgctcacttgtgacacccccgaagaggatggcatcacatggactttagatcagtccagcgaggtgctgggaagcggcaagactttaacaatccaagttaaggagttcggagacgccggacagtatacttgtcacaagggcggcgaagtgctgagccattctttactgttattacataagaaggaggacggcatctggagcaccgacatcctcaaggaccagaaggagcccaaaaacaagaccttcttacgttgtgaggccaaaaactattccggcagatttacttgttggtggctgacaaccatcagcacagatttaacctttagcgtgaagagctctcgtggaagcagcgaccctcaaggtgtgacatgtggagccgccaccctcagcgccgaaagggttcgtggcgataataaagaatacgagtatagcgtggagtgccaagaagacagcgcttgccccgctgccgaagaatctttacccatcgaggtgatggtcgacgccgtccacaagctgaagtacgaaaactacacatcctccttcttcatcagagatatcatcaagcccgatccccccaagaatctgcagctgaaacctttaaagaattctcgtcaagttgaagtgagctgggagtatcccgacacttggtccaccccccactcctacttcagcctcaccttctgcgtgcaagttcaaggcaaatctaaaagggagaagaaggacagagtgtttacagacaagaccagcgctaccgtgatctgtcgtaagaacgcctccatcagcgtgagggctcaagatcgttattacagctccagctggagcgaatgggcttccgtgccttgttctgtgcccggtgtgggcgtgcccggcgtgggcagaaatctccccgtcgccacccccgatcccggaatgttcccttgtttacaccattcccagaatttattaagggccgtgagcaacatgctgcaaaaggccagacagacactggagttctacccttgcaccagcgaggagattgatcacgaggacatcaccaaggacaaaaccagcacagtggaggcttgtttacctctggaactcaccaagaacgagtcttgtttaaactccagagagaccagctttatcaccaacggcagctgtttagcctctcgtaaaaccagcttcatgatggctttatgtttaagcagcatctacgaggatttaaagatgtaccaagttgaattcaagaccatgaacgccaagttattaatggatcccaagaggcagatctttttagaccagaacatgctggccgtgatcgacgagctgatgcaagctttaaacttcaactccgaaaccgtgccccagaaaagcagcctcgaggagcccgacttctacaaaacaaaaattaagctgtgcatcttattacacgcctttaggattcgtgccgtgaccatcgatcgtgtcatgagctatttaaacgcttcctag

SEQ ID NO: 52 - protein, 538 aa, Artificial Sequence
  source: /note=“Description of Artificial Sequence Synthetic polypeptide”
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 53 - DNA, 1617 bp, Artificial Sequence
  source: /note=“Description of Artificial Sequence Synthetic polynucleotide”
atgtgtcctcagaagctgacaattagttggttcgccattgttctcctcgtctcaccacttatggcaatgtgggaactggaaaaagacgtttacgtggttgaggttgattggactcccgacgccccaggtgaaaccgtaaatctgacctgtgacacacctgaggaagatgacatcacctggaccagtgaccagcgccacggagtgatagggagtgggaagacattgacaatcactgtaaaagagtttctcgacgctggacaatacacatgtcacaagggaggcgaaacactctctcatagccacttgctgttgcacaagaaggagaacggcatatggtccacagagattctcaagaatttcaagaataaaaccttcctcaagtgcgaagcccctaactatagtgggaggtttacttgctcatggctcgtgcagcgcaatatggacctgaagttcaacattaaaagttctagctcatccccagactcacgcgccgtgacatgtgggatggcaagcctcagcgccgagaaagtaacattggatcagagagactatgagaaatattccgtgagctgccaagaagacgttacctgtccaaccgccgaggagaccctgcctatagagttggctcttgaggcaaggcaacaaaacaaatacgagaactattccacaagttttttcataagagacataatcaagcctgaccccccaaaaaatctccagatgaagccactgaaaaattctcaagtcgaggttagttgggaatatccagattcttggtcaactccacacagttatttctctcttaagtttttcgttcgcatacagcggaaaaaggagaagatgaaggaaaccgaggaagggtgcaatcaaaaaggagctttcttggtagagaaaacatccactgaagtccagtgcaaaggtggtaacgtgtgcgtccaggctcaggatagatactataactcatcctgctcaaaatgggcttgcgtcccatgccgcgtacgaagcgtgccaggagtaggagttccaggtgttggccgggtcatacctgtaagtggtcccgctcggtgtctctctcagtctcggaatctccttaaaacaacagacgacatggtaaaaacagcccgggagaaattgaaacactactcttgcacagccgaagacattgatcatgaagacatcacccgagaccagacctcaacactgaaaacatgccttccactcgagctgcacaagaatgaatcatgcctggctactcgcgagacaagcagtactacccgcggtagctgcctcccaccccaaaagacatctcttatgatgaccctgtgcctcggctctatctacgaggaccttaagatgtaccaaaccgaattccaagccatcaacgctgcattgcaaaatcataatcatcaacagataattctcgataagggtatgctcgtagctatagatgagcttatgcagtcacttaaccacaacggtgagaccttgaggcaaaaaccacccgtgggagaagcagatccatatcgagtgaagatgaagctctgcatcttgttgcatgcctttagtaccagggtggtaactataaacagggtaatgggttacctctcttccgcctga

SEQ ID NO: 54 - protein, 538 aa, Artificial Sequence; identical to SEQ ID NO: 52
  source: /note=“Description of Artificial Sequence Synthetic polypeptide”

SEQ ID NO: 55 - protein, 10 aa, Artificial Sequence
  source: /note=“Description of Artificial Sequence Synthetic peptide”
VPGVGVPGVG

SEQ ID NO: 56 - protein, 15 aa, Artificial Sequence
  source: /note=“Description of Artificial Sequence Synthetic peptide”
GGGGSGGGGSGGGGS

SEQ ID NO: 57 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 58 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 60 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 61 - protein, 5 aa, Artificial Sequence
  source: /note=“Description of Artificial Sequence Synthetic peptide”
  source: /note=“See specification as filed for detailed description of substitutions and preferred embodiments”
GGGGS

SEQ ID NO: 62 - protein, 150 aa, Artificial Sequence
  source: /note=“Description of Artificial Sequence Synthetic polypeptide”
  SITE (1)..(150): /note=“This sequence may encompass 1-30 ′Gly Gly Gly Gly Ser′ repeating units”
  source: /note=“See specification as filed for detailed description of substitutions and preferred embodiments”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 63 - protein, 5 aa, Artificial Sequence
  source: /note=“Description of Artificial Sequence Synthetic peptide”
TNGIS

SEQ ID NO: 64 - protein, 17 aa, Artificial Sequence
  source: /note=“Description of Artificial Sequence Synthetic peptide”
WISAGGGPTNYAQKLQG

SEQ ID NO: 65 - protein, 8 aa, Artificial Sequence
  source: /note=“Description of Artificial Sequence Synthetic peptide”
GLYGTKDA

SEQ ID NO: 66 - protein, 13 aa, Artificial Sequence
  source: /note=“Description of Artificial Sequence Synthetic peptide”
SGGGSNIGSNAVN

SEQ ID NO: 67 - protein, 7 aa, Artificial Sequence
  source: /note=“Description of Artificial Sequence Synthetic peptide”
YNDLLPS

SEQ ID NO: 68 - protein, 11 aa, Artificial Sequence
  source: /note=“Description of Artificial Sequence Synthetic peptide”
AAWDDNLSAYV